(19) United States
(12) Patent Application Publication
(10) Pub. No.: US 2026/0337162 A1
(43) Pub. Date: Oct. 29, 2026

(54) TRYPTAMINE COMPOUNDS, COMPOSITIONS, AND METHODS OF USE

(71) Applicant: Cybin IRL Limited, Dublin (IE)

(72) Inventors: Alex Nivorozhkin, West Roxbury, MA (US); Joshua A. Hartsel, Lake Forest, CA (US); Clinton E. Canal, Atlanta, GA (US); Tina A. Mueller, West Palm Beach, FL (US); Kenneth L. Avery, Merrimack, NH (US); Geoffrey B. Varty, Annandale, NJ (US); Francesco G. Salituro, Marlborough, MA (US); Michael Palfreyman, St. Petersburg, MA (US)

(73) Assignee: Cybin IRL Limited, Dublin (IE)

(21) Appl. No.: 19/106,551

(22) PCT Filed: Aug. 23, 2023
(86) PCT No.: PCT/EP2023/073122

§ 371 Date: Feb. 25, 2025

Related U.S. Application Data
(60) Provisional application No. 63/402,650, filed on Aug. 31, 2022.

(51) Int. Cl. C07D 209/16; A61K 31/4045; A61P 25/18

(52) U.S. Cl. CPC C07D 209/16; A61K 31/4045; A61P 25/18

(57) ABSTRACT

The present disclosure relates to tryptamine compounds, and pharmaceutically acceptable salts, stereoisomers, tautomers, solvates, polymorphs, or prodrugs thereof, to pharmaceutical compositions, and in some embodiments, to serotonin 5-HT₂ receptor agonists and uses in the treatment of diseases such as those diseases associated with a 5-HT₂ receptor.

### CROSS-REFERENCE

This application claims priority to U.S. Provisional Application No. 63/402,650, filed on Aug. 31, 2022, incorporated by reference herein in its entirety.

### FIELD

The present disclosure relates generally to tryptamine compounds, compositions, and, in some embodiments, to serotonin 5-HT₂ receptor agonists and uses in the treatment of diseases associated with a 5-HT₂ receptor.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

There are three, closely related subtypes of serotonin 5-HT₂ receptors (5-HT₂RS), 5-HT_{2A}, 5-HT_{2B}, and 5-HT_{2C}, and they are primary targets of classic serotonergic psychedelics, such as lysergic acid diethylamide (LSD), 2,5-dimethoxy-4-bromoamphetamine (DOB), N,N-dimethyltryptamine (DMT), 5-methoxy-N,N-dimethyltryptamine (5-MeO-DMT), psilocybin and its active dephosphorylated form, psilocin. Each subtype is expressed in a unique pattern in mammals (both in peripheral tissues and in the central nervous system), and when stimulated, produces unique biochemical, physiological, and behavioral effects. Activation of 5-HT_{2A}RS, for example, predominantly mediates psychedelic effects and elicits anti-inflammatory effects, whereas activation of 5-HT_{2C}RS reduces feeding behavior. Chronic activation of 5-HT_{2B}RS, however, has been linked to valvular heart disease (VHD), a life-threatening adverse event (AE).

Classic serotonergic psychedelics and entactogens have been actively investigated by the research and medical community to alleviate a multitude of central nervous system (CNS) disorders (Reiff, C. M., Richman, E. E., Nemeroff, C. B., Carpenter, L. L., Widge, A. S., Rodriguez, C. I., Kalin, N. H., and McDonald, W. M., 2020, Psychedelics and Psychedelic-Assisted Psychotherapy, Am J Psychiatry 177, 391-410), such as: (i) post-traumatic stress disorder (PTSD)(Jerome, L., Feduccia, A. A., Wang, J. B., Hamilton, S., Yazar-Klosinski, B., Emerson, A., Mithoefer, M. C., and Doblin, R., 2020, Long-term follow-up outcomes of MDMA-assisted psychotherapy for treatment of PTSD: a longitudinal pooled analysis of six phase 2 trials, Psychopharmacology (Berl) 237, 2485-2497), (ii) major depressive disorder (MDD), (iii) treatment-resistant depression (TRD)(Goldberg, S. B., Pace, B. T., Nicholas, C. R., Raison, C. L., and Hutson, P. R., 2020, The experimental effects of psilocybin on symptoms of anxiety and depression: A meta-analysis, Psychiatry Res 284, 112749), (iv) obsessive-compulsive disorder (OCD)(Moreno, F. A., Wiegand, C. B., Taitano, E. K., and Delgado, P. L., 2006, Safety, tolerability, and efficacy of psilocybin in 9 patients with obsessive-compulsive disorder, J Clin Psychiatry 67, 1735-1740), (v) social anxiety disorder (ClinicalTrials.gov, number NCT02008396), (vi) substance use disorders such as alcohol use disorder, opioid use disorder, amphetamine use disorder, nicotine use disorder, and cocaine use disorder, (vii) anorexia nervosa, (viii) bulimia nervosa (ClinicalTrials.gov, numbers NCT04454684 and NCT04052568), (ix) Alzheimer's disease (ClinicalTrials.gov, number NCT04123314), and (x) cluster headache and migraine (Nichols, D. E., 2016, Psychedelics, Pharmacol Rev 68, 264-355; Johnson, M. W., Hendricks, P. S., Barrett, F. S., and Griffiths, R. R., 2019, Classic psychedelics: An integrative review of epidemiology, therapeutics, mystical experience, and brain network function, Pharmacol Ther 197, 83-102; Sewell, R. A., Halpern, J. H., and Pope, H. G., Jr., 2006, Response of cluster headache to psilocybin and LSD, Neurology 66, 1920-1922; ClinicalTrials.gov, number NCT04218539).

These drugs have also been investigated to alleviate conditions of the autonomic nervous system (ANS), including pulmonary disorders (e.g., asthma and chronic obstructive pulmonary disorder (COPD) and cardiovascular disorders (e.g., atherosclerosis), among others (Nichols, D. E., Johnson, M. W., and Nichols, C. D., 2017, Psychedelics as Medicines: An Emerging New Paradigm, Clin Pharmacol Ther 101, 209-219; Flanagan, T. W., Sebastian, M. N., Battaglia, D. M., Foster, T. P., Cormier, S. A., and Nichols, C. D., 2019, 5-HT₂ receptor activation alleviates airway inflammation and structural remodeling in a chronic mouse asthma model, Life Sci 236, 116790; Flanagan, T. W., Sebastian, M. N., Battaglia, D. M., Foster, T. P., Maillet, E. L., and Nichols, C. D., 2019, Activation of 5-HT₂ Receptors Reduces Inflammation in Vascular Tissue and Cholesterol Levels in High-Fat Diet-Fed Apolipoprotein E Knockout Mice, Sci Rep 9, 13444; Sexton, J. D., Nichols, C. D., and Hendricks, P. S., 2019, Population Survey Data Informing the Therapeutic Potential of Classic and Novel Phenethylamine, Tryptamine, and Lysergamide Psychedelics, Front Psychiatry 10, 896).

Despite high potencies at serotonin 5-HT₂RS (in some cases sub-nanomolar affinities), the therapeutic value of certain tryptamine psychedelics has been hampered by their accelerated metabolism in the liver and gastrointestinal tract most notably by monoamine oxidase (MAO) enzymes. For example, DMT is not orally active—it is converted to inactive metabolites before sufficient brain penetration can occur. Likewise, 5-MeO-DMT lacks oral bioavailability and is instead ordinarily vaporized and inhaled to produce psychedelic effects. Psilocybin and psilocin also undergo MAO-mediated metabolism in vivo, which is believed to contribute to significant patient—to —patient pharmacokinetic variability. Additionally, the duration of action of certain tryptamine psychedelics is so short, 5-15 minutes in the case of DMT and 5-MeO-DMT, as to limit their use in effective therapies.

### SUMMARY

In view of the forgoing, there is a need for novel tryptamine psychedelics, such as those with affinity for different conformations or populations of 5-HT receptors and improved pharmacokinetic properties—that are orally bioavailable, brain penetrable, and have a clinically effective duration of action. There is a further need for efficient, more convenient, and controllable tryptamine formulations that provide controlled drug exposure and maintain drug concentrations in the safe and efficacious range.

One object of the present disclosure is to provide novel tryptamine compounds which meet these criteria, as well as compositions thereof, and methods of using the same to treat diseases, e.g., those associated with a serotonin 5-HT₂ receptor. More specifically, the present disclosure provides novel fluorinated tryptamine analogs and compositions thereof, that can be used to treat neuropsychiatric disorders, central nervous system (CNS) disorders, neurodegenerative diseases, and other disorders, such as those associated with inflammation.

Thus, the present disclosure provides:
(1) A compound of Formula (I), or a pharmaceutically acceptable salt, stereoisomer, tautomer, solvate, polymorph, or prodrug thereof,

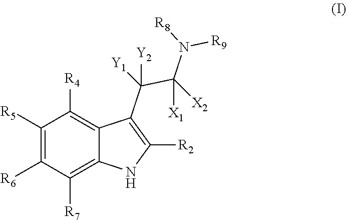

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₄ is selected from the group consisting of hydrogen, deuterium, hydroxyl, unsubstituted or substituted alkoxy, and —OPO₃H₂;
   R₅ is selected from the group consisting of hydrogen, deuterium, hydroxyl, unsubstituted alkyl, alkyl substituted with one or more deuterium, unsubstituted alkoxy, alkoxy substituted with one or more deuterium, unsubstituted alkylthio, alkylthio substituted with one or more deuterium, —OR_{f}, and —SR_{f};
   R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₈ is selected from the group consisting of hydrogen, unsubstituted alkyl, an alkyl substituted with one or more deuterium, and R_{f},
   R₉ is selected from the group consisting of unsubstituted alkyl, an alkyl substituted with one or more deuterium, R_{f}, —S(O) R_{f}, and —S(O)₂R_{f};
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₅, R₈, and R₉ comprises the fluoroalkyl group, R_{f}, and/or R₈ and R₉ together with the nitrogen atom attached thereto are joined to form the heterocycloalkyl substituted with at least one fluorine; and
   with the provisos that (i) R₉ is not —CH₂CF₃ when X₁, X₂, Y₁, Y₂, R₂, R₄, R₅, R₆, and R₇ are hydrogen and R₈ is hydrogen or methyl, and (ii) R₈ and R₉ are not joined with the nitrogen atom attached thereto to form a 4,4-difluoropiperidinyl group when X₁, X₂, Y₁, Y₂, R₂, R₄, R₅, R₆, and R₇ are hydrogen.
(2) The compound of (1), wherein X₁, X₂, Y₁, and Y₂ are hydrogen.
(3) The compound of (1) or (2), wherein R₂ is hydrogen.
(4) The compound of any one of (1) to (3), wherein R₄ is hydrogen.
(5) The compound of any one of (1) to (3), wherein R₄ is hydroxyl.
(6) The compound of any one of (1) to (5), wherein R₅ is selected from the group consisting of hydrogen, unsubstituted alkoxy, alkoxy substituted with one or more deuterium, unsubstituted alkylthio, alkylthio substituted with one or more deuterium, —OR_{f}, and —SR_{f}.
(7) The compound of any one of (1) to (6), wherein R₅ is hydrogen.
(8) The compound of any one of (1) to (6), wherein R₅ is selected from the group consisting of unsubstituted alkoxy, alkoxy substituted with one or more deuterium, and —OR_{f}.
(9) The compound of any one of (1) to (6), wherein R₅ is selected from the group consisting of unsubstituted alkylthio, alkylthio substituted with one or more deuterium, and —SR_{f}.
(10) The compound of any one of (1) to (6), wherein R₅ is —OR_{f} or —SR_{f}.
(11) The compound of (10), wherein n is 0.
(12) The compound of any one of (1) to (11), wherein R₆ and R₇ are hydrogen.
(13) The compound of any one of (1) to (12), wherein R₈ is hydrogen or an unsubstituted C₁-C₆ alkyl.
(14) The compound of any one of (1) to (12), wherein R₈ is R_{f}.
(15) The compound of (14), wherein H^{x} is hydrogen and n is 2.
(16) The compound of any one of (1) to (15), wherein R₉ is an unsubstituted C₁-C₆ alkyl.
(17) The compound of any one of (1) to (15), wherein R₉ is R_{f}.
(18) The compound of (17), wherein H^{x} is hydrogen and n is 2.
(19) The compound of any one of (1) to (15), wherein R₉ is —S(O) R_{f} or —S(O)₂R_{f}.
(20) The compound of (19), wherein n is 0.
(21) The compound of any one of (1) to (20), which is selected from the group consisting of

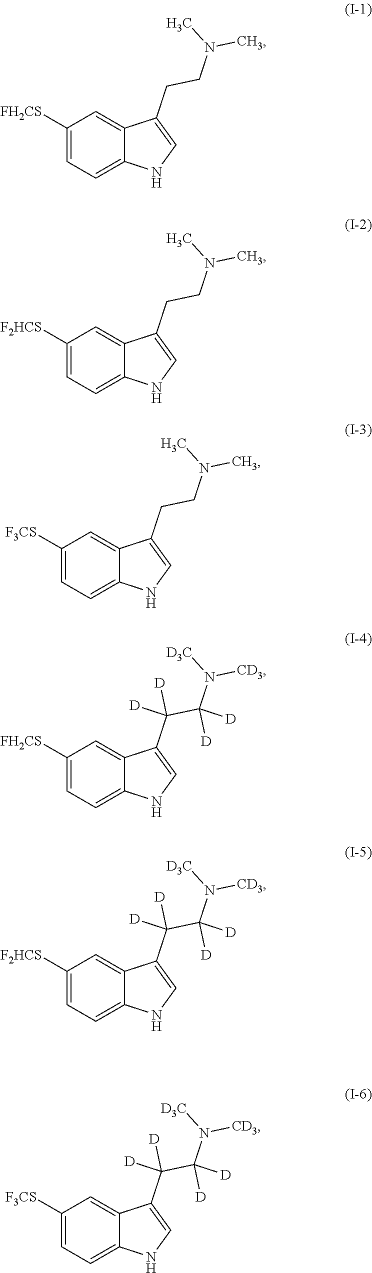

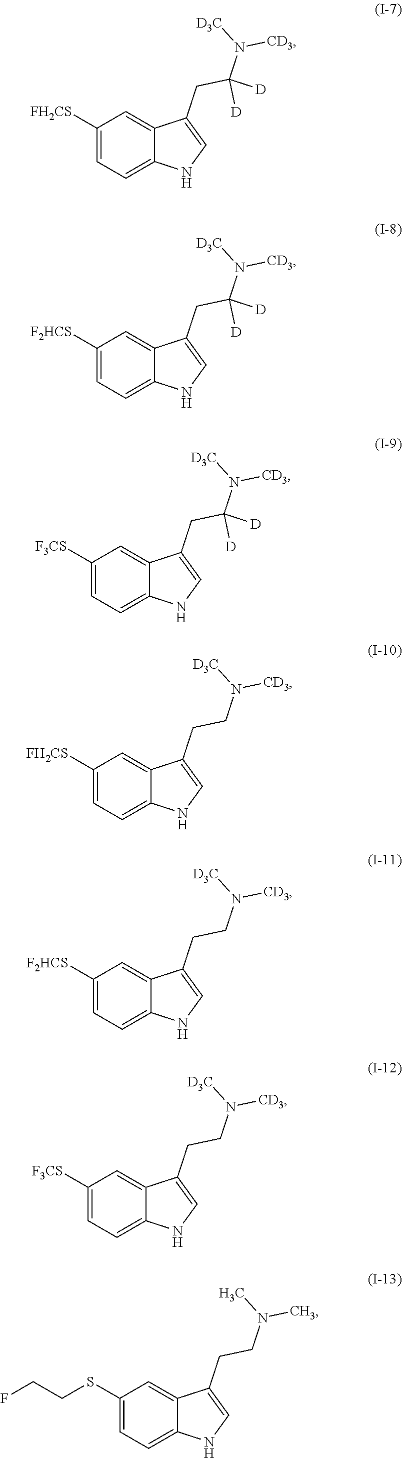

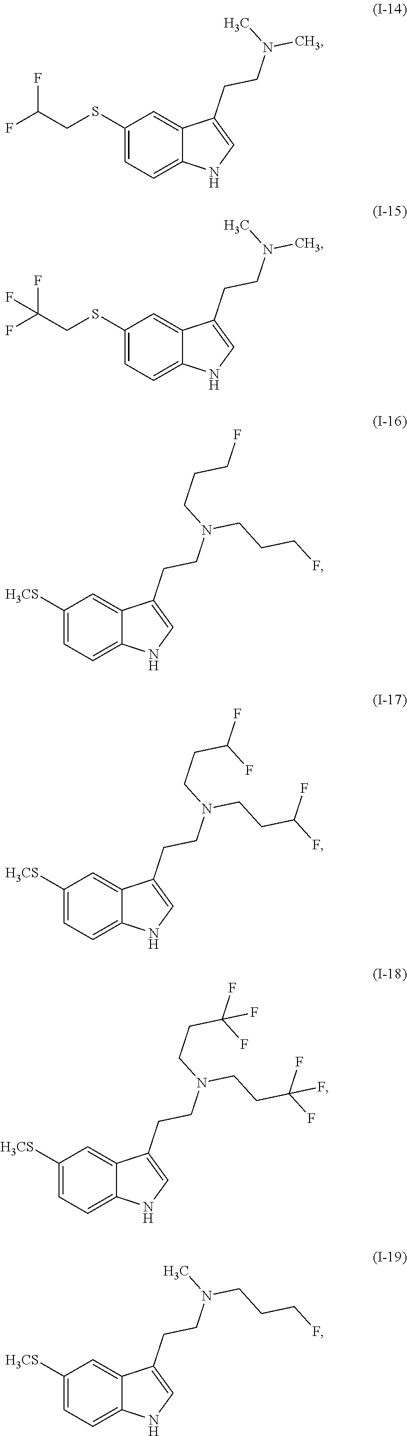

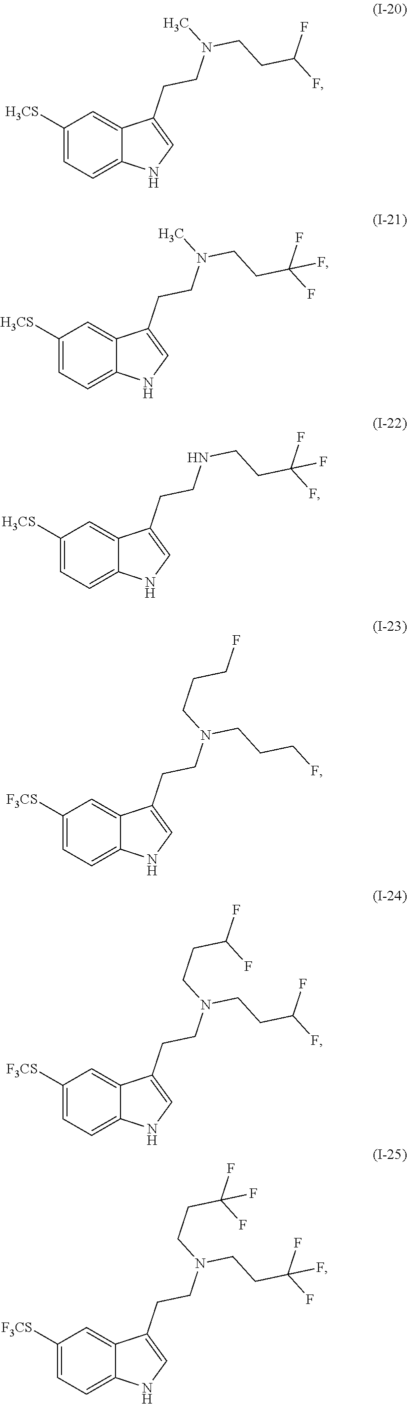

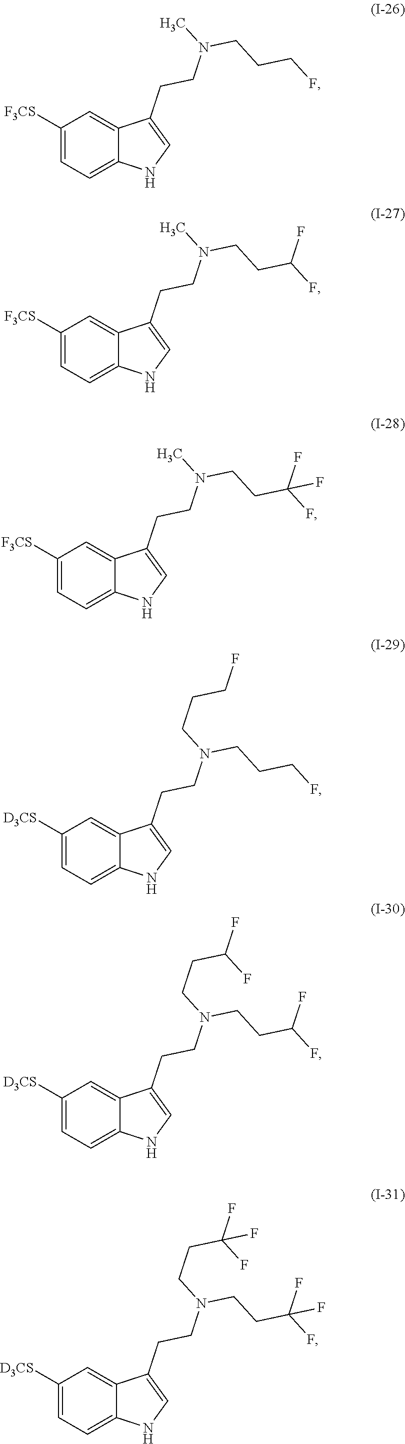

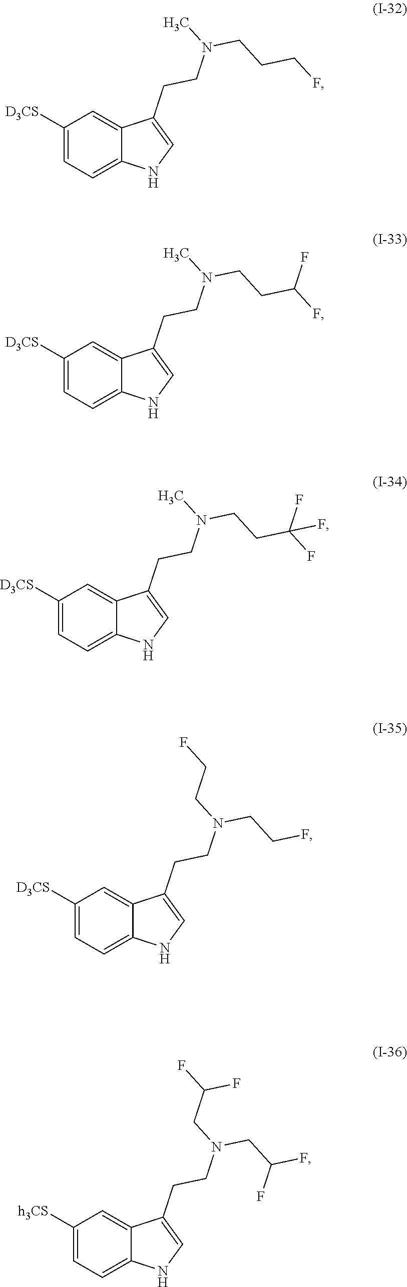

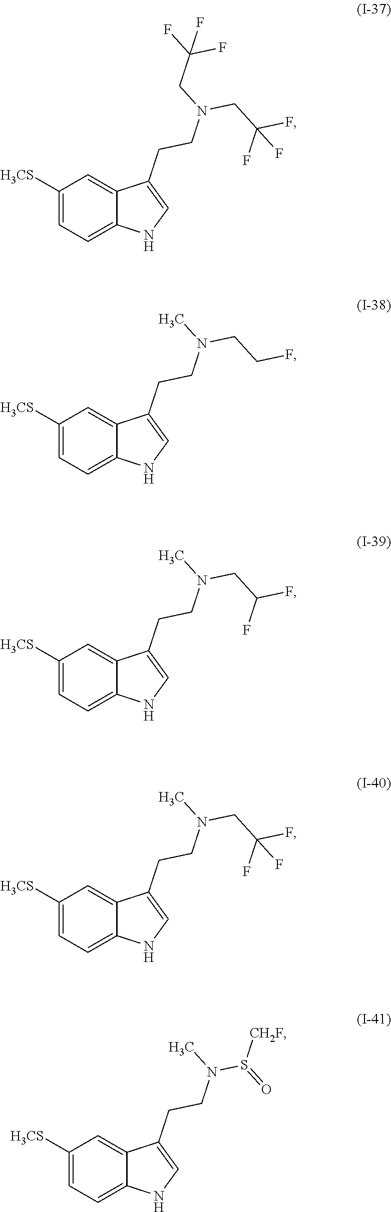

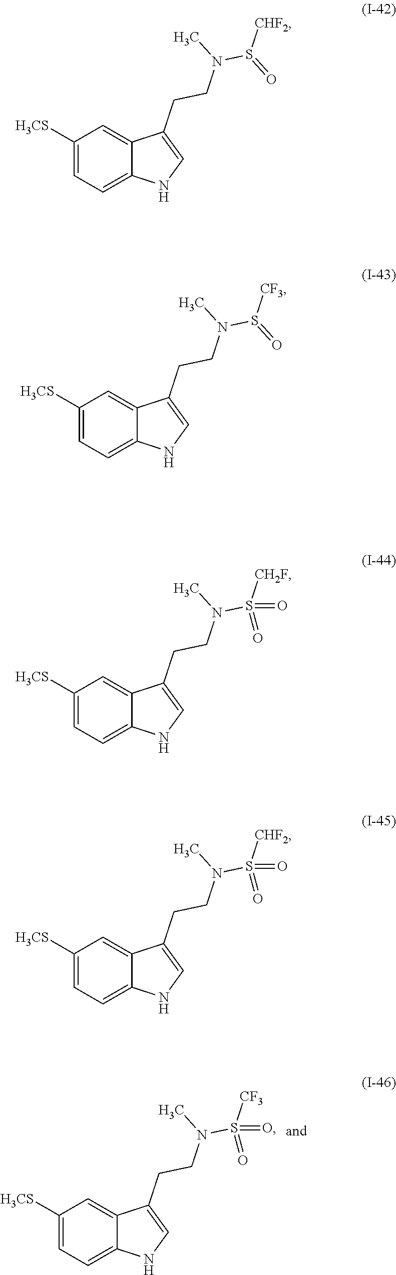

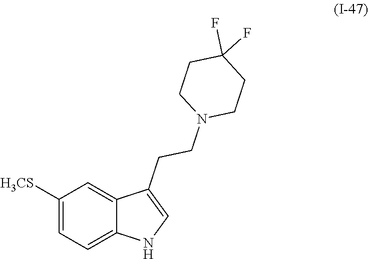

or a pharmaceutically acceptable salt, stereoisomer, tautomer, solvate, polymorph, or prodrug thereof.
(22) The compound of (1), having a structure of Formula (II), or a pharmaceutically acceptable salt, stereoisomer, tautomer, solvate, polymorph, or prodrug thereof,

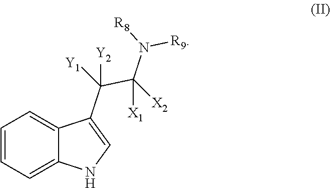

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₈ is selected from the group consisting of hydrogen, unsubstituted alkyl, an alkyl substituted with one or more deuterium, and R_{f};
   R₉ is selected from the group consisting of unsubstituted alkyl, an alkyl substituted with one or more deuterium, R_{f}, —S(O) R_{f}, and —S(O)₂R_{f},
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₈ and R₉ comprises the fluoroalkyl group, R_{f}, or R₈ and R₉ together with the nitrogen atom attached thereto are joined to form the heterocycloalkyl substituted with at least one fluorine; and
   with the provisos that (i) R₉ is not —CH₂CF₃ when X₁, X₂, Y₁, and Y₂ are hydrogen and R₈ is hydrogen or methyl and (ii) R₈ and R₉ are not joined with the nitrogen atom attached thereto to form a 4,4-difluoropiperidinyl group when X₁, X₂, Y₁, and Y₂ are hydrogen.
(23) The compound of (22), wherein X₁, X₂, Y₁, and Y₂ are hydrogen.
(24) The compound of (22) or (23), wherein R₈ is hydrogen or an unsubstituted C₁-C₆ alkyl.
(25) The compound of (22) or (23), wherein R₈ is R_{f}.
(26) The compound of (25), wherein H^{x} is hydrogen and n is 2.
(27) The compound of any one of (22) to (26), wherein R₉ is R_{f}.
(28) The compound of (27), wherein H^{x} is hydrogen and n is 2.
(29) The compound of any one of (22) to (26), wherein R₉ is —S(O) R_{f} or —S(O)₂R_{f}.
(30) The compound of (29), wherein n is 0.
(31) The compound of any one of (22) to (30), which is selected from the group consisting of

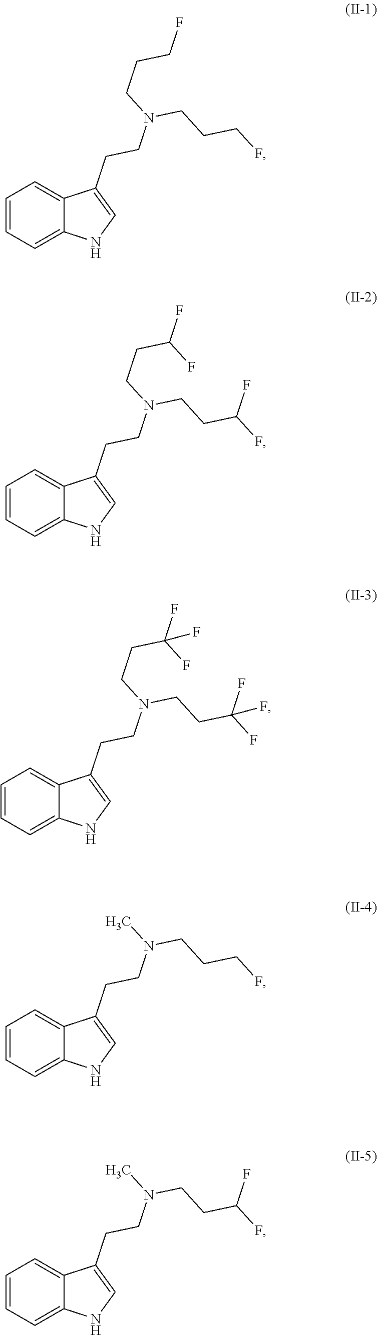 [SMILES: FCCCN(CCCF)CCc1c[nH]c2ccccc12 ; FC(F)CCN(CCc1c[nH]c2ccccc12)CCC(F)F ; FC(F)(F)CCN(CCc1c[nH]c2ccccc12)CCC(F)(F)F ; CN(CCCF)CCc1c[nH]c2ccccc12 ; CN(CCc1c[nH]c2ccccc12)CCC(F)F]

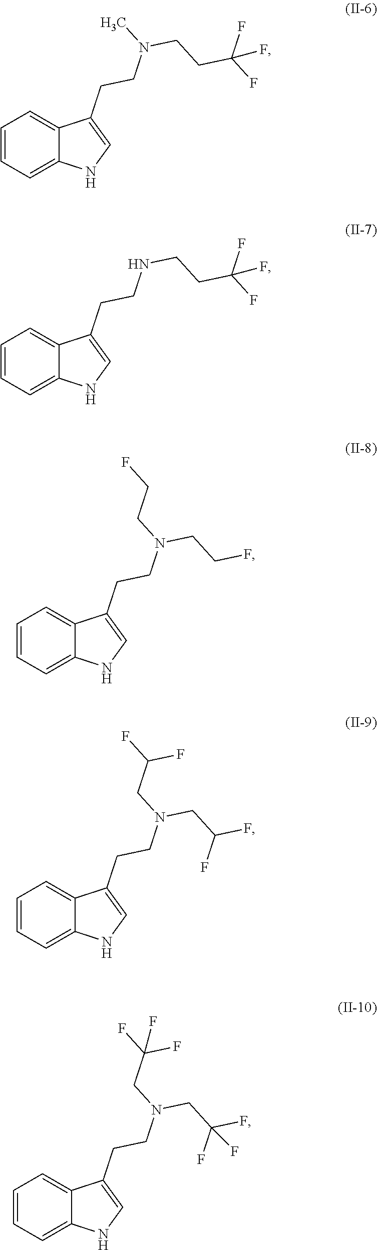 [SMILES: CN(CCc1c[nH]c2ccccc12)CCC(F)(F)F ; FC(F)(F)CCNCCc1c[nH]c2ccccc12 ; FCCN(CCF)CCc1c[nH]c2ccccc12 ; FC(F)CN(CCc1c[nH]c2ccccc12)CC(F)F ; FC(F)(F)CN(CCc1c[nH]c2ccccc12)CC(F)(F)F]

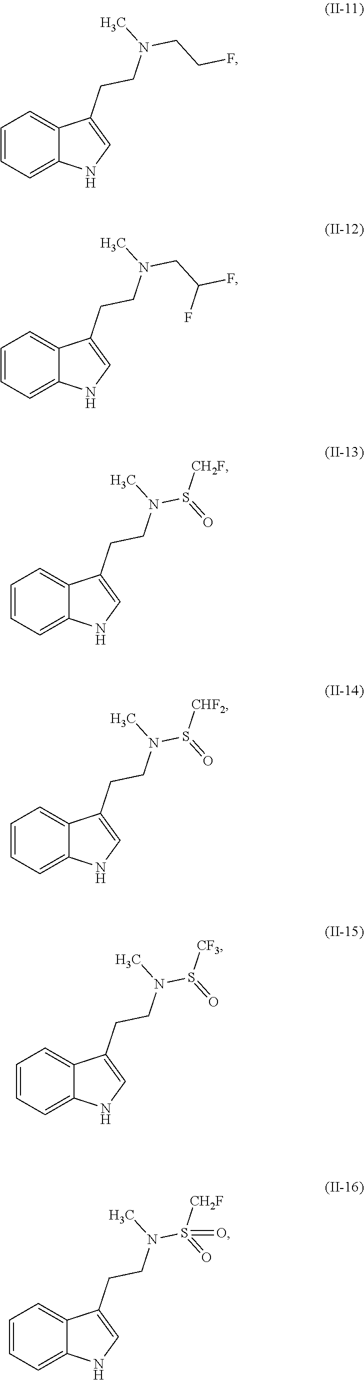

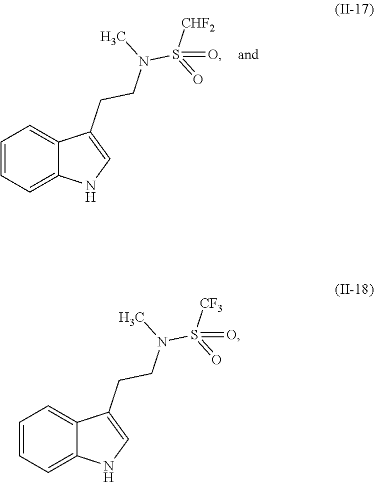 [SMILES: CN(CCc1c[nH]c2ccccc12)S(=O)(=O)C(F)F ; "and" ; CN(CCc1c[nH]c2ccccc12)S(=O)(=O)C(F)(F)F]

or a pharmaceutically acceptable salt, stereoisomer, tautomer, solvate, polymorph, or prodrug thereof.
(32) The compound of (1), having a structure of Formula (III), or a pharmaceutically acceptable salt, stereoisomer, tautomer, solvate, polymorph, or prodrug thereof,

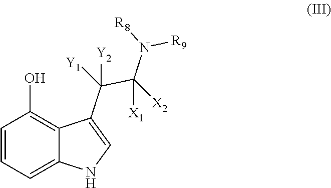

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₈ is selected from the group consisting of hydrogen, unsubstituted alkyl, an alkyl substituted with one or more deuterium, and R_{f};
   R₉ is selected from the group consisting of unsubstituted alkyl, an alkyl substituted with one or more deuterium, R_{f}, —S(O) R_{f}, and —S(O)₂R_{f};
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₈ and R₉ comprises the fluoroalkyl group, R_{f}, or R₈ and R₉ together with the nitrogen atom attached thereto are joined to form the heterocycloalkyl substituted with at least one fluorine.
(33) The compound of (32), wherein X₁, X₂, Y₁, and Y₂ are hydrogen.
(34) The compound of (32) or (33), wherein R₈ is hydrogen or an unsubstituted C₁-C₆ alkyl.
(35) The compound of (32) or (33), wherein R₈ is R_{f}.
(36) The compound of (35), wherein H^{x} is hydrogen and n is 2.
(37) The compound of any one of (32) to (36), wherein R₉ is R_{f}.
(38) The compound of (37), wherein H^{x} is hydrogen and n is 2.
(39) The compound of any one of (32) to (36), wherein R₉ is —S(O) R_{f} or —S(O)₂R_{f}.
(40) The compound of (39), wherein n is 0.
(41) The compound of any one of (32) to (40), which is selected from the group consisting of

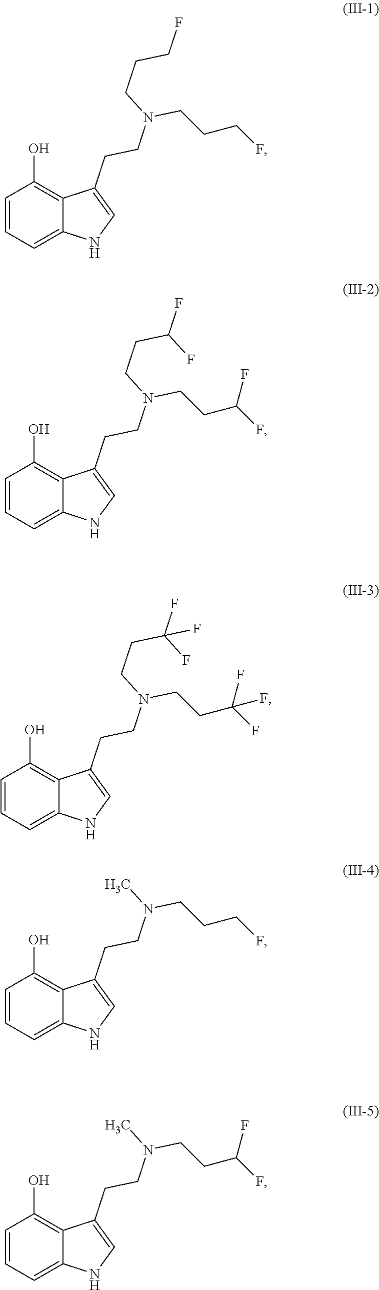 [SMILES: Oc1cccc2[nH]cc(CCN(CCCF)CCCF)c12 ; Oc1cccc2[nH]cc(CCN(CCC(F)F)CCC(F)F)c12 ; Oc1cccc2[nH]cc(CCN(CCC(F)(F)F)CCC(F)(F)F)c12 ; CN(CCCF)CCc1c[nH]c2cccc(O)c12 ; CN(CCc1c[nH]c2cccc(O)c12)CCC(F)F]

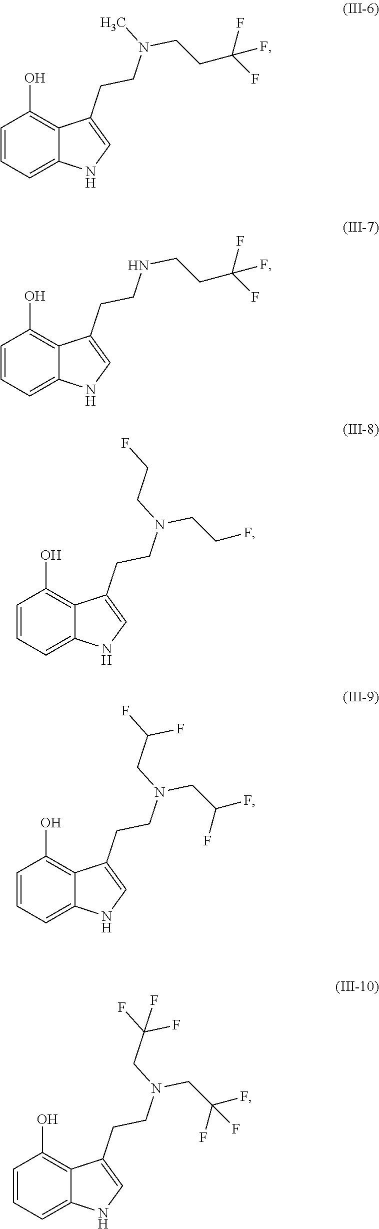 [SMILES: CN(CCc1c[nH]c2cccc(O)c12)CCC(F)(F)F ; Oc1cccc2[nH]cc(CCNCCC(F)(F)F)c12 ; Oc1cccc2[nH]cc(CCN(CCF)CCF)c12 ; Oc1cccc2[nH]cc(CCN(CC(F)F)CC(F)F)c12 ; Oc1cccc2[nH]cc(CCN(CC(F)(F)F)CC(F)(F)F)c12]

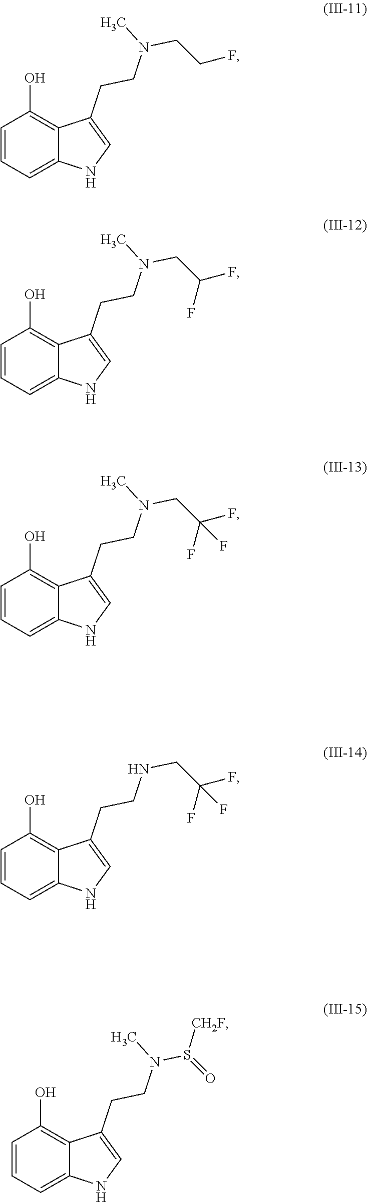

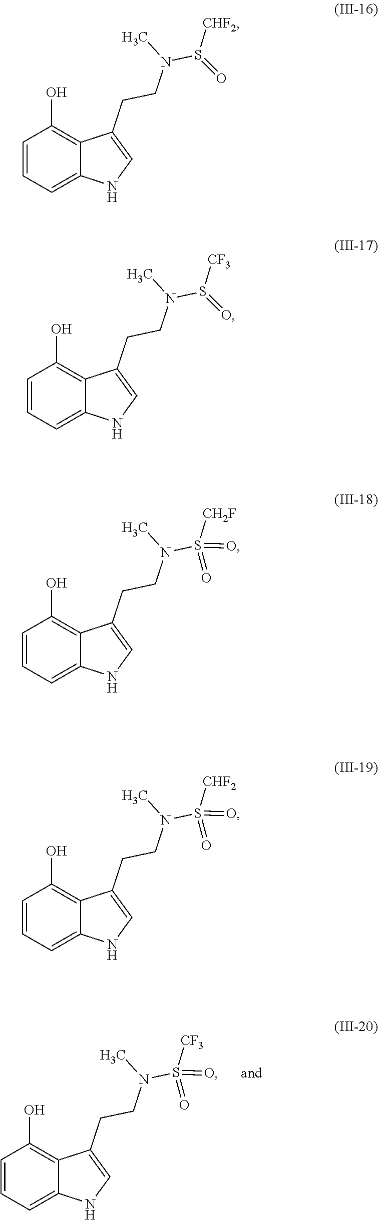

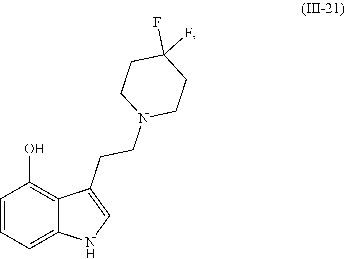 [SMILES: Oc1cccc2[nH]cc(CCN3CCC(F)(F)CC3)c12]

or a pharmaceutically acceptable salt, stereoisomer, tautomer, solvate, polymorph, or prodrug thereof.
(42) The compound of (1), having a structure of Formula (IV), or a pharmaceutically acceptable salt, stereoisomer, tautomer, solvate, polymorph, or prodrug thereof,

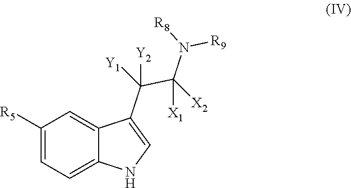

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₈ is selected from the group consisting of unsubstituted alkoxy, alkoxy substituted with one or more deuterium, and —OR_{f},
   R₈ is selected from the group consisting of hydrogen, unsubstituted alkyl, an alkyl substituted with one or more deuterium, and R_{f};
   R₉ is selected from the group consisting of unsubstituted alkyl, an alkyl substituted with one or more deuterium, R_{f}, —S(O) R_{f}, and —S(O)₂R_{f};
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₅, R₈, and R₉ comprises the fluoroalkyl group, R_{f}, and/or R₈ and R₉ together with the nitrogen atom attached thereto are joined to form the heterocycloalkyl substituted with at least one fluorine.
(43) The compound of (42), wherein X₁, X₂, Y₁, and Y₂ are hydrogen.
(44) The compound of (42) or (43), wherein R₅ is an unsubstituted C₁-C₆ alkoxy group.
(45) The compound of (42) or (43), wherein R₅ is —OR_{f}.
(46) The compound of (45), wherein n is 0.
(47) The compound of any one of (42) to (46), wherein R₈ is hydrogen or an unsubstituted C₁-C₆ alkyl.
(48) The compound of any one of (42) to (46), wherein R₈ is R_{f}.
(49) The compound of (48), wherein H^{x} is hydrogen and n is 2.
(50) The compound of any one of (42) to (49), wherein R₉ is an unsubstituted C₁-C₆ alkyl.
(51) The compound of any one of (42) to (49), wherein R₉ is R_{f}.
(52) The compound of (51), wherein Ha is hydrogen and n is 2.
(53) The compound of any one of (42) to (49), wherein R₉ is —S(O) R_{f} or —S(O)₂R_{f}.
(54) The compound of (53), wherein n is 0.
(55) The compound of any one of (42) to (54), which is selected from the group consisting of

[chemical structure]

[chemical structure]

or a pharmaceutically acceptable salt, stereoisomer, tautomer, solvate, polymorph, or prodrug thereof.
(56) The compound of (1), having a structure of Formula (V), or a pharmaceutically acceptable salt, stereoisomer, tautomer, solvate, polymorph, or prodrug thereof,

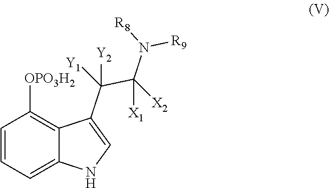

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₈ is selected from the group consisting of hydrogen, unsubstituted alkyl, an alkyl substituted with one or more deuterium, and R_{f},
   R₉ is selected from the group consisting of unsubstituted alkyl, an alkyl substituted with one or more deuterium, R_{f}, —S(O) R_{f}, and —S(O)₂R_{f}, or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₈ and R₉ comprises the fluoroalkyl group, R_{f}, or R₈ and R₉ together with the nitrogen atom attached thereto are joined to form the heterocycloalkyl substituted with at least one fluorine.
(57) The compound of (56), wherein X₁, X₂, Y₁, and Y₂ are hydrogen.
(58) The compound of (56) or (57), wherein R₈ is hydrogen or an unsubstituted C₁-C₆ alkyl.
(59) The compound of (56) or (57), wherein R₈ is R_{f}.
(60) The compound of (59), wherein H^{x} is hydrogen and n is 2.
(61) The compound of any one of (56) to (60), wherein R₉ is R_{f}.
(62) The compound of (61), wherein H^{x} is hydrogen and n is 2.
(63) The compound of any one of (56) to (60), wherein R₉ is —S(O) R_{f} or —S(O)₂R_{f}.
(64) The compound of (63), wherein n is 0.
(65) The compound of any one of (56) to (64), which is selected from the group consisting of

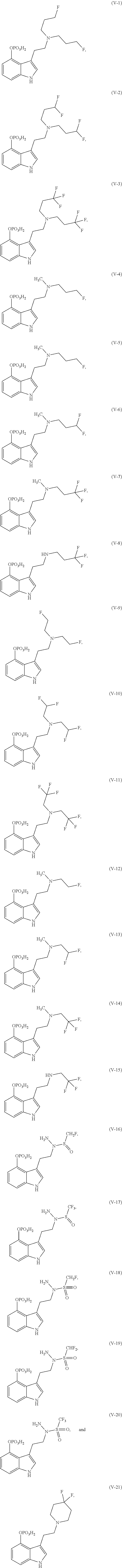

or a pharmaceutically acceptable salt, stereoisomer, tautomer, solvate, polymorph, or prodrug thereof.
(66) A pharmaceutical composition comprising:
   a compound of Formula (I), or a pharmaceutically acceptable salt, stereoisomer, tautomer, solvate, polymorph, or prodrug thereof; and
   a pharmaceutically acceptable vehicle,

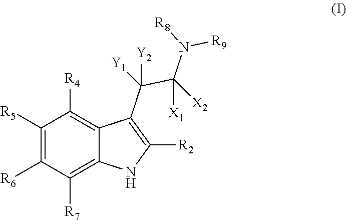

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₄ is selected from the group consisting of hydrogen, deuterium, hydroxyl, unsubstituted or substituted alkoxy, and —OPO₃H₂;
   R₅ is selected from the group consisting of hydrogen, deuterium, hydroxyl, unsubstituted alkyl, alkyl substituted with one or more deuterium, unsubstituted alkoxy, alkoxy substituted with one or more deuterium, unsubstituted alkylthio, alkylthio substituted with one or more deuterium, —OR_{f}, and —SR_{f},
   R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₈ is selected from the group consisting of hydrogen, unsubstituted alkyl, an alkyl substituted with one or more deuterium, and R_{f},
   R₉ is selected from the group consisting of unsubstituted alkyl, an alkyl substituted with one or more deuterium, R_{f}, —S(O) R_{f}, and —S(O)₂R_{f};
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine.
(67) The pharmaceutical composition of (66), which is adapted for oral administration.
(68) A method of treating a subject with a disease or disorder, comprising:
   administering to the subject a therapeutically effective amount of a compound of Formula (I), or a pharmaceutically acceptable salt, stereoisomer, tautomer, solvate, polymorph, or prodrug thereof,

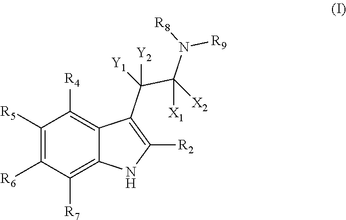

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₄ is selected from the group consisting of hydrogen, deuterium, hydroxyl, unsubstituted or substituted alkoxy, and —OPO₃H₂;
   R₅ is selected from the group consisting of hydrogen, deuterium, hydroxyl, unsubstituted alkyl, alkyl substituted with one or more deuterium, unsubstituted alkoxy, alkoxy substituted with one or more deuterium, unsubstituted alkylthio, alkylthio substituted with one or more deuterium, —OR_{f}, and —SR_{f};
   R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₈ is selected from the group consisting of hydrogen, unsubstituted alkyl, an alkyl substituted with one or more deuterium, and R_{f},
   R₉ is selected from the group consisting of unsubstituted alkyl, an alkyl substituted with one or more deuterium, R_{f}, —S(O) R_{f}, and —S(O)₂R_{f};
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine.
(69) A method of treating a subject with a disease or disorder associated with a serotonin 5-HT₂ receptor, comprising:
   administering to the subject a therapeutically effective amount of a compound of Formula (I), or a pharmaceutically acceptable salt, stereoisomer, tautomer, solvate, polymorph, or prodrug thereof,

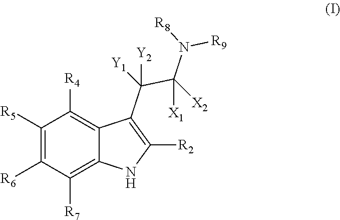

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₄ is selected from the group consisting of hydrogen, deuterium, hydroxyl, unsubstituted or substituted alkoxy, and —OPO₃H₂;
   R₅ is selected from the group consisting of hydrogen, deuterium, hydroxyl, unsubstituted alkyl, alkyl substituted with one or more deuterium, unsubstituted alkoxy, alkoxy substituted with one or more deuterium, unsubstituted alkylthio, alkylthio substituted with one or more deuterium, —OR_{f}, and —SR_{f};
   R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₈ is selected from the group consisting of hydrogen, unsubstituted alkyl, an alkyl substituted with one or more deuterium, and R_{f},
   R₉ is selected from the group consisting of unsubstituted alkyl, an alkyl substituted with one or more deuterium, R_{f}, —S(O) R_{f}, and —S(O)₂R_{f};
   or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine.
(70) The method of (69), wherein the disease or disorder is a neuropsychiatric disease or disorder or an inflammatory disease or disorder.
(71) The method of (69), wherein the disease or disorder is a central nervous system (CNS) disorder.
(72) The method of (71), wherein the central nervous system (CNS) disorder is at least one selected from the group consisting of major depressive disorder (MDD), treatment-resistant depression (TRD), post-traumatic stress disorder (PTSD), bipolar and related disorders, obsessive-compulsive disorder (OCD), generalized anxiety disorder (GAD), social anxiety disorder, a substance use disorder, an eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, a paraphilic disorder, sexual dysfunction, peripheral neuropathy, and obesity.
(73) The method of (71), wherein the central nervous system (CNS) disorder is major depressive disorder (MDD).
(74) The method of (71), wherein the central nervous system (CNS) disorder is treatment-resistant depression (TRD).
(75) The method of (71), wherein the central nervous system (CNS) disorder is generalized anxiety disorder (GAD).
(76) The method of (71), wherein the central nervous system (CNS) disorder is social anxiety disorder.
(77) The method of (71), wherein the central nervous system (CNS) disorder is obsessive-compulsive disorder (OCD).
(78) The method of (71), wherein the central nervous system (CNS) disorder is cluster headaches or migraine.
(79) The method of (71), wherein the central nervous system (CNS) disorder is a substance use disorder.
(80) The method of (79), wherein the substance use disorder is alcohol use disorder.
(81) The method of (79), wherein the substance use disorder is nicotine use disorder.
(82) The method of (69), wherein the disease or disorder is an autonomic nervous system (ANS) condition.
(83) The method of (69), wherein the disease or disorder is a pulmonary disorder.
(84) The method of (69), wherein the disease or disorder is a cardiovascular disorder.
(85) The method of any one of (69) to (84), wherein the compound is administered orally to the subject.
(86) The method of any one of (69) to (85), wherein the compound is administered intraorally to the subject.
(87) The method of any one of (69) to (86), wherein the compound is administered to the subject at a psychedelic dose of about 0.083 mg/kg to about 5 mg/kg.
(88) The method of (87), wherein the compound is administered at the psychedelic dose once per week or less over a treatment course.
(89) The method of (69) to (86), wherein the compound is administered to the subject at a sub-psychedelic dose of about 0.00001 mg/kg to less than about 0.083 mg/kg.
(90) The method of (89), wherein the compound is administered at the sub-psychedelic dose once per day or more over a treatment course.
(91) Use of a compound of Formula (I), including any of Formula (II) through (V) or a pharmaceutically acceptable salt, stereoisomer, tautomer, solvate, polymorph, or prodrug thereof, for treating a subject with a disease or disorder associated with a serotonin 5-HT₂ receptor,

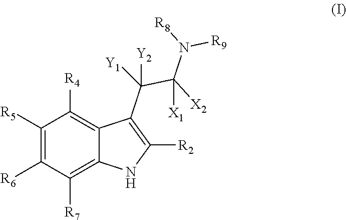

wherein:
   X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   Y₁ and Y₂ are independently selected from the group consisting of hydrogen and deuterium;
   R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₄ is selected from the group consisting of hydrogen, deuterium, hydroxyl, unsubstituted or substituted alkoxy, and —OPO₃H₂;
   R₈ is selected from the group consisting of hydrogen, deuterium, hydroxyl, unsubstituted alkyl, alkyl substituted with one or more deuterium, unsubstituted alkoxy, alkoxy substituted with one or more deuterium, unsubstituted alkylthio, alkylthio substituted with one or more deuterium, —OR_{f}, and —SR_{f};
   R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
   R₈ is selected from the group consisting of hydrogen, unsubstituted alkyl, an alkyl substituted with one or more deuterium, and R_{f},
   R₉ is selected from the group consisting of unsubstituted alkyl, an alkyl substituted with one or more deuterium, R_{f}, —S(O) R_{f}, and —S(O)₂R_{f}, or alternatively R₈ and R₉ together with the nitrogen atom attached thereto are optionally joined to form a heterocycloalkyl substituted with at least one fluorine; and
   R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
   wherein at least one of R₅, R₈, and R₉ comprises the fluoroalkyl group, R_{f}, and/or R₈ and R₉ together with the nitrogen atom attached thereto are joined to form the heterocycloalkyl substituted with at least one fluorine.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIG. 1 shows examples of general synthetic routes which can be used to access the compounds of the present disclosure, LG=leaving group, e.g., chloride, bromide, tosylate, etc;

FIG. 2 shows a synthetic route to compound I-3;

FIG. 3 shows a synthetic route to compound II-2;

FIG. 4 shows a synthetic route to compound II-3;

FIG. 5 shows a synthetic route to compound II-6;

FIG. 6 shows a synthetic route to compound II-10;

FIG. 7 shows a synthetic route to compound II-12;

FIG. 8 shows a synthetic route to compound II-15;

FIG. 9 shows a synthetic route to compound II-19;

FIG. 10 shows a synthetic route to compound II-20;

FIG. 11 shows a synthetic route to compound II-21;

FIG. 12 shows a synthetic route to compound III-2;

FIG. 13 shows a synthetic route to compound III-3;

FIG. 14 shows a synthetic route to compound III-6;

FIG. 15 shows a synthetic route to compound III-10;

FIG. 16 shows a synthetic route to compound III-12;

FIG. 17 shows a synthetic route to compound III-13;

FIG. 18 shows a synthetic route to compound III-14;

FIG. 19 shows a synthetic route to compound IV-3;

FIG. 20 shows a synthetic route to compound IV-21;

FIG. 21 shows a synthetic route to compound IV-40;

FIG. 22 is a graph of agonist-labeled human serotonin 5-HT_{2A} receptor radioligand ([³H]LSD) competition binding using compound II-19 and serotonin (5-HT); results are from four independent experiments with 5-HT tested in duplicate (N=8 replicates) and II-19 in triplicate (N=12 replicates); data analyzed using a one-site fit Kᵢ model;

FIG. 23 is a graph of agonist-labeled human serotonin 5-HT_{2A} receptor radioligand ([³H]LSD) competition binding using compounds II-10 and II-20 and serotonin (5-HT); results are from four independent experiments with 5-HT tested in duplicate (N=08 replicates) and II-10 and II-20 in triplicate (N=12 replicates); data analyzed using a one-site fit Kᵢ model;

FIG. 24 is a graph of agonist-labeled human serotonin 5-HT_{2A} receptor radioligand ([³H]LSD) competition binding using compounds II-6 and III-6 and serotonin (5-HT); results are from three independent experiments with 5-HT tested in duplicate (N=06 replicates) and II-6 and III-6 in triplicate (N=09 replicates); data analyzed using a one-site fit Kᵢ model;

FIG. 25 is a graph showing the effects of compounds II-6 and III-6 (10 mg/kg PO) compared to positive control (±) 2,5-dimethoxy-4-iodoamphetamine (DOI, 10 mg/kg PO) on the serotonin 5-HT_{2A} receptor-dependent head-twitch response (HTR) in adult, male C57Bl/6J mice;

FIG. 26 is a graph showing 5-HT_{2A} receptor functional assay results, as percent maximal 5-HT change from baseline; compounds were tested in triplicate at 500 nM and 50 AM concentrations;

FIG. 27 is a graph showing the dose-response 5-HT_{2A} functional assay results as a percent of control agonist response for compound II-2; tested in duplicate at the indicated concentrations and the EC₅₀ values were determined by non-linear regression analysis of the concentration-response curves generated with mean replicate values using Hill equation curve fitting;

FIG. 28 is a graph showing the dose-response 5-HT_{2A} functional assay results as a percent of control agonist response for compounds III-2 and IV-21; tested in duplicate at the indicated concentrations and the EC₅₀ values were determined by non-linear regression analysis of the concentration-response curves generated with mean replicate values using Hill equation curve fitting; and

FIG. 29 is a graph showing the dose-response 5-HT_{2B} functional assay results as a percent of control agonist response for compounds III-2 and IV-21; tested in duplicate at the indicated concentrations and the EC₅₀ values were determined by non-linear regression analysis of the concentration-response curves generated with mean replicate values using Hill equation curve fitting.

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

“Alkyl” refers to monovalent saturated aliphatic hydrocarbyl groups having from 1 to 10 carbon atoms and such as 1 to 6 carbon atoms, or 1 to 5, or 1 to 4, or 1 to 3, or 1 to 2 carbon atoms. This term includes, by way of example, linear and branched hydrocarbyl groups such as methyl(CH₃—), ethyl(CH₃CH₂—), n-propyl(CH₃CH₂CH₂—), isopropyl((CH₃)₂CH—), n-butyl(CH₃CH₂CH₂CH₂—), isobutyl((CH₃)₂CHCH₂—), sec-butyl((CH₃)(CH₃CH₂) CH—), t-butyl(t-Bu)((CH₃)₃C—), n-pentyl(CH₃CH₂CH₂CH₂CH₂—), and neopentyl((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 10 substituents selected from the group consisting of deuterium, alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups having from 1 to 6, including, for example, 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃) CH₂—), and the like.

“Substituted alkylene” refers to an alkylene group having from 1 to 3 hydrogens replaced with substituents as described for carbons in the definition of “substituted” below. The term “alkane” refers to alkyl group and alkylene group, as defined herein. The term “alkylaminoalkyl”, “alkylaminoalkenyl” and “alkylaminoalkynyl” refers to the groups R′NHR″—where R′ is alkyl group as defined herein and R″ is alkylene, alkenylene or alkynylene group as defined herein.

The term “alkaryl” or “aralkyl” refers to the groups-alkylene-aryl andsubstituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O—where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O—wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups-alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group-alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms, for example 2 to 4 carbon atoms and having at least 1, for example from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms, for example, 2 to 3 carbon atoms and having at least 1 and for example, from 1 to 2 sites of triple bond unsaturation. Examples of such alkynyl groups include acetylenyl (—C═CH), and propargyl (—CH₂C═CH).

The term “substituted alkynyl” refers to an alkynyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)

“Acylamino” refers to the groups —NR²⁰C(O)alkyl, —NR²⁰C(O) substituted alkyl, NR²⁰C(O)cycloalkyl, —NR²⁰C(O) substituted cycloalkyl, NR²⁰C(O)cycloalkenyl, —NR²⁰C(O) substituted cycloalkenyl, —NR²⁰C(O)alkenyl, NR²⁰C(O) substituted alkenyl, —NR²⁰C(O)alkynyl, —NR²⁰C(O) substituted alkynyl, —NR²⁰C(O) aryl, —NR²⁰C(O) substituted aryl, —NR²⁰C(O) heteroaryl, —NR²⁰C(O) substituted heteroaryl, —NR²⁰C(O) heterocyclic, and —NR²⁰C(O) substituted heterocyclic, wherein R₂₀ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R₂₂ independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₂₁ and R₂₂ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R₂₃ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O—wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R₂₁ and R₂₂ independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R₂₁ and R₂₂ are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R₂₁ and R₂₂ independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₂₁ and R₂₂ are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxyl, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.
“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
“Cyano” or “nitrile” refers to the group-CN.
“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclohexyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from deuterium, alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and for example, from 1 to 2 double bonds.

The term “substituted cycloalkenyl” refers to cycloalkenyl groups having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxyl, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups-alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero-ring atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, aziridine, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholinyl), 1,1-dioxothiomorpholine, pyrrolidine, tetrahydrofuran, benzo[d][1,3]oxathiole, benzo[d][1,3]dioxole, and the like.

Unless otherwise constrained by the definition for the heterocyclic substituent, such heterocyclic groups can be optionally substituted with 1 to 5, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic —S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, deuterium, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂ (M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S) OR⁷⁰, —C(O)NR⁸⁰R⁸⁰, —C(NR⁷⁰) NR⁸⁰R⁸⁰, —OC(O)R⁷⁰, —OC(S)R⁷⁰, —OC(O)OM⁺, —OC(O)OR⁷⁰, —OC(S) OR⁷⁰, —NR⁷⁰C(O)R⁷⁰, —NR⁷⁰C(S)R⁷⁰, —NR⁷⁰CO₂—NR⁷⁰CO₂R⁷⁰, M⁺, —NR⁷⁰C(S) OR⁷⁰, —NR⁷⁰C(O)NR⁸⁰R⁸⁰, —NR⁷⁰C(NR⁷⁰)R⁷⁰ and —NR⁷⁰C(NR⁷⁰) NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the disclosure and the other a typical counter ion such as chloride, or two ionized compounds disclosed herein can serve as counter ions for such divalent alkali earth ions, or a doubly ionized compound of the disclosure can serve as the counter ion for such divalent alkali earth ions). As specific examples, —NR⁸⁰R⁸⁰ is meant to include-NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, deuterium, —R⁶⁰, halo, —OM⁺, —OR⁷⁰, —SR⁷⁰, —S⁻M⁺, —NR⁸⁰R⁸⁰ trihalomethyl, —CF₃, —CN, —OCN, —SCN, —NO, —NO₂, —N₃, —SO₂R⁷⁰, —SO₃ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S) OR⁷⁰, —C(O)NR⁸⁰R⁸⁰, —C(NR⁷⁰) NR⁸⁰R⁸⁰, —OC(O)R⁷⁰, —OC(S)R⁷⁰, —OCO₂⁻M⁺, —OCO₂R⁷⁰, —NR⁷⁰C(O)R⁷⁰, —OC(S) OR⁷⁰, —NR⁷⁰C(S)R⁷⁰, —NR⁷⁰CO₂⁻M⁺, —NR⁷⁰CO₂R⁷⁰, —NR⁷⁰C(S) OR⁷⁰, —NR⁷⁰C(O)NR⁸⁰R⁸⁰, —NR⁷⁰C(NR⁷⁰)R⁷⁰ and —NR⁷⁰C(NR⁷⁰) NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl unless groups are, otherwise specified, —R⁶⁰, —O⁻M⁺, —OR⁷⁰, —SR⁷⁰, —S⁻M⁺, NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰) OM⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S) OR⁷⁰, —C(O)NR⁸⁰R⁸⁰, —C(NR⁷⁰) NR⁸⁰R⁸⁰, —OC(O)R⁷⁰, —OC(S)R⁷⁰, —OC(O)OR⁷⁰, —OC(S) OR⁷⁰, —NR⁷⁰C(O)R⁷⁰, —NR⁷⁰C(S)R⁷⁰, —NR⁷⁰C(O)OR⁷⁰, —NR⁷⁰C(S) OR⁷⁰, —NR⁷⁰C(O)NR⁸⁰R⁸⁰, —NR⁷⁰C(NR⁷⁰)R⁷⁰ and —NR⁷⁰C(NR⁷⁰) NR⁸⁰R⁸⁰, where R⁶⁰, R⁷⁰R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein, unless specified otherwise. In such cases, the maximum number of such substitutions is three. For example, serial substitutions of substituted aryl groups specifically contemplated herein are limited to substituted aryl-(substituted aryl)-substituted aryl. However, substituent groups defined as e.g., polyethers may contain serial substitution greater than three, e.g., —O—(CH₂CH₂O), —H, where n can be 1, 2, 3, or greater.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)—O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

When it is stated that a substituent or group “comprise(s) a/the fluoroalkyl group” (which is sometimes denoted as R_{f} herein) it is to be understood that the substituent or group may itself be the fluoroalkyl group, or the substituent or group may contain a fluoroalkyl group within in its chemical structure, provided that the presence of the fluoroalkyl group is consistent with the other requirements set forth of the substituent or group being discussed. For example, when substituent “—R” is defined to comprise a fluoroalkyl group, it is to be understood that-R may be itself a fluoroalkyl group (e.g., —CF₃), or a group containing the fluoroalkyl group (e.g., —SCF₃) that is consistent with the other requirements set forth of —R.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, malonate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like. The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis —trans isomers, E and Z isomers, enantiomers, and diastereomers.

“Tautomer” refers to alternate forms of a molecule that differ only in electronic bonding of atoms and/or in the position of a proton, such as enol-keto, imine-enamine, and neutral/zwitterionic tautomers, or the tautomeric forms of heteroaryl groups containing a —N═C(H)—NH-ring atom arrangement, such as pyrazoles, imidazoles, benzimidazoles, triazoles, and tetrazoles. Other tautomeric ring atom arrangements are also possible. For example, compounds containing an acid and a base group within the same molecule depicted in neutral form may exist also in a zwitterionic form, as is the case for amino acid/ammonium carboxylate tautomers. Thus, compounds of the present disclosure, e.g., compounds of Formula (I), Formula (V), etc., which are depicted to contain both amino and dihydrogen phosphate (—OPO₃H₂) functionality in neutral form may also exist in zwitterionic form as the ammonium monohydrogen phosphate zwitterion. A given chemical formula or name shall encompass all tautomeric forms thereof, insofar as they exist.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, e.g., an ester, a phosphate ester, etc. but is converted in vivo to an active compound, for example, by hydrolysis to a free carboxylic acid or free hydroxyl group. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxyl, free amino or free mercapto group, respectively. Examples of prodrugs include, but are not limited to, acetate, formate, benzoate, and dihydrogen phosphate derivatives of an alcohol, or acetamide, formamide and benzamide derivatives of an amine functional group in the active compound and the like.

A “crystalline” solid is a type of solid whose fundamental three-dimensional structure consists of a highly regular pattern of atoms or molecules—with long range order-forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity—less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of an internal standard. Other characterization techniques, such as modulated differential scanning calorimetry (mDSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

It will be appreciated that the compounds herein can exist in different salt, solvate, stereoisomer, tautomer, crystalline/amorphous (or polymorphic) forms, and the present disclosure is intended to include all permutations thereof, such as a solvate of a pharmaceutically acceptable salt of a stereoisomer of the subject compound.

The term “stable,” “stability,” and the like, as used herein includes chemical stability and solid state (physical) stability. The term “chemical stability” means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g., hydration, dehydration, solvatization, desolvatization, crystallization, recrystallization or solid-state phase transition). As used herein, the term “composition” is equivalent to the term “formulation.”

A “vapor” is a solid substance in the gas phase at a temperature lower than its critical temperature, meaning that the vapor can be condensed to a liquid by increasing the pressure on it without reducing the temperature.

An “aerosol”, as used herein, is a suspension of fine solid particles or liquid droplets in a gas phase (e.g., air, oxygen, helium, nitrous oxide, and other gases, as well as mixtures thereof). A “mist”, as used herein, is a subset of aerosols, differing from a vapor, and is a dispersion of liquid droplets (liquid phase) suspended in the gas phase (e.g., air, oxygen, helium, and mixtures thereof). The liquid droplets of an aerosol or mist can comprise a drug moiety dissolved in an aqueous liquid, organic solvent, or a mixture thereof. The gas phase of an aerosol or mist can comprise air, oxygen, helium, or other gases, including mixtures thereof. Mists do not comprise solid particulates. Aerosols and mists of the present disclosure can be generated by any suitable methods and devices, examples of which are set forth herein, e.g., through use of an inhaler or nebulizer.

As used herein, the term “inhalation session” describes a dosing event whereby the subject inhales a given dose of drug, irrespective of the number of breadths needed to inhale the given dose. For example, a subject prescribed to take 10 mg of a drug twice a day would undertake two inhalation sessions, each inhalation session providing 10 mg of the drug. The length of time and the number of breaths for each inhalation session would be dependent on factors such as the inhalation device used, the amount of drug that is drawn per breath, the concentration of the drug in the dosage form, the subject's breathing pattern, etc.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating one or more symptoms of the disease or medical condition in a patient. In an embodiment, prophylactic treatment can result in preventing the disease or medical condition from occurring, in a subject.

A “patient” or “subject,” used interchangeably herein, can be any mammal including, for example, a human or a non-human subject. A patient or subject can have a condition to be treated or can be susceptible to a condition to be treated.

As used herein, and unless otherwise specified, the terms “prevent,” “preventing” and “prevention” refer to the prevention of the onset, recurrence or spread of a disease, disorder, or condition, or of one or more symptoms thereof. The terms encompass the inhibition or reduction of a symptom of the particular disease, disorder, or condition. Subjects with familial history of a disease, disorder, or condition, in particular, are candidates for preventive regimens in certain embodiments. In addition, subjects who have a history of recurring symptoms are also potential candidates for the prevention. In this regard, the term “prevention” may be interchangeably used with the term “prophylactic treatment.”

“Therapeutically effective amount” refers to an amount of a compound(s) sufficient to treat a specified disorder or disease or one or more of its symptoms and/or to prevent the occurrence of the disease or disorder (prophylactically effective amount).

As used herein, and unless otherwise specified, a “prophylactically effective amount” of an active agent, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The language “toxic spikes” is used herein to describe neurological spikes in concentration of any compound described herein that would produce side-effects of sedation or psychotomimetic effects, e.g., hallucination, dizziness, and nausea; which can not only have immediate repercussions, but also effect treatment compliance. In particular, side effects may become more pronounced at blood concentration levels of about 250, 300, 400, 500 ng/L or more.

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, attention deficit disorder, attention deficit hyperactivity disorder, bipolar and manic disorders, depression, or any combinations thereof.

“Inflammatory conditions” or “inflammatory disease,” as used herein, refers broadly to chronic or acute inflammatory diseases, including, but not limited to, rheumatic diseases (e.g., rheumatoid arthritis, osteoarthritis, psoriatic arthritis) spondyloarthropathies (e.g., ankylosing spondylitis, reactive arthritis, Reiter's syndrome), crystal arthropathies (e.g., gout, pseudogout, calcium pyrophosphate deposition disease), multiple sclerosis, Lyme disease, polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, glaucoma, and cataracts.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

### Compounds

### Formula (I)

Disclosed herein is a compound of Formula (I), or a pharmaceutically acceptable salt, stereoisomer, tautomer, solvate, polymorph, or prodrug thereof,

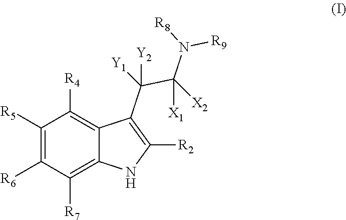

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ is selected from the group consisting of hydrogen, deuterium, hydroxyl, unsubstituted or substituted alkoxy, and —OPO₃H₂;
R₅ is selected from the group consisting of hydrogen, deuterium, hydroxyl, unsubstituted alkyl, alkyl substituted with one or more deuterium, unsubstituted alkoxy, alkoxy substituted with one or more deuterium, unsubstituted alkylthio, alkylthio substituted with one or more deuterium, —OR_{f}, and —SR_{f};
R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₈ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f};
or alternatively R₈ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine.
X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is an unsubstituted or substituted alkyl (e.g., an unsubstituted or substituted a C₁-C₆ alkyl). In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, X₁ and/or X₂ is an unsubstituted or substituted alkenyl, e.g., an unsubstituted or substituted allyl. In some embodiments, X₁ and/or X₂ is an unsubstituted or substituted alkynyl. In some embodiments, X₁ and/or X₂ is an unsubstituted or substituted C₃-C₁₀ cycloalkyl. In some embodiments, X₁ and/or X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, X₁ and/or X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, X₁ and/or X₂ is an unsubstituted or substituted heterocycloalkyl. In some embodiments, X₁ and/or X₂ is an unsubstituted or substituted aryl. In some embodiments, X₁ and/or X₂ is an unsubstituted or substituted heteroaryl.
Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₂ is hydrogen. In some embodiments, R₂ is deuterium. In some embodiments, R₂ is a halogen, e.g., fluoro, chloro, bromo, and iodo. In some embodiments, R₂ is an unsubstituted or substituted alkyl (e.g., an unsubstituted or substituted a C₁-C₆ alkyl). In some embodiments, R₂ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₂ is a substituted alkyl (e.g., a C₁-C₆ alkyl). When R₂ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₂ is an unsubstituted or substituted alkenyl, e.g., an unsubstituted or substituted allyl. In some embodiments, R₂ is an unsubstituted or substituted alkynyl. In some embodiments, R₂ is an unsubstituted or substituted C₃-C₁₀ cycloalkyl. In some embodiments, R₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₂ is an unsubstituted or substituted heterocycloalkyl. In some embodiments, R₂ is an unsubstituted or substituted aryl. In some embodiments, R₂ is an unsubstituted or substituted heteroaryl.

In some embodiments, R₄ is hydrogen. In some embodiments, R₄ is deuterium. In some embodiments, R₄ is hydroxyl. In some embodiments, R₄ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₄ is a substituted alkoxy. When R₄ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc. In some embodiments, R₄ is —OPO₃H₂.

In some embodiments, R₈ is hydrogen. In some embodiments, R₅ is deuterium. In some embodiments, R₅ is hydroxyl. In some embodiments, R₅ is an unsubstituted alkyl (e.g., an unsubstituted C₁-C₆ alkyl), examples of which include, but are not limited to, methyl, ethyl, n-propyl, isopropyl, n-butyl, isobutyl, sec-butyl, t-butyl, n-pentyl, neopentyl, and hexyl. In some embodiments, R₅ is methyl. In some embodiments, R₅ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃. In some embodiments, R₅ is an unsubstituted alkoxy group, such as an unsubstituted C₁-C₆ alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is an alkoxy group substituted with one or more deuterium. The alkoxy group may contain one, or more than one, deuterium substituent. For example, when the alkoxy group is a C₁ alkoxy group (i.e., methoxy group), the deuterium substituted C₁ alkoxy group may be —OCDH₂, —OCD₂H, and —OCD₃. In some embodiments, R₅ is an unsubstituted alkylthio group, examples of which include, but are not limited to, methylthio, ethylthio, n-propylthio, isopropylthio, n-butylthio, isobutylthio, sec-butylthio, t-butylthio, n-pentylthio, neopentylthio, and hexylthio. In some embodiments, R₅ is an alkylthio group substituted with one or more deuterium. The alkylthio group may contain one, or more than one, deuterium substituent. For example, when the alkylthio group is a C₁ alkylthio group (i.e., a methylthio group), the deuterium substituted C₁ alktlthio group may be —SCDH₂, —SCD₂H, and —SCD₃.

In some embodiments, R₅ is —OR_{f}, examples of which include, but are not limited to, —OCH₂F, —OCHF₂, —OCF₃, —OCH₂CH₂F, —OCH₂CHF₂, —OCH₂CF₃, —OCH₂CH₂CH₂F, —OCH₂CH₂CHF₂, —OCH₂CH₂CF₃, —OCH₂CH₂CH₂CH₂F, —OCH₂CH₂CH₂CHF₂, and —OCH₂CH₂CH₂CF₃, with particular mention being made to —OCH₂F, —OCHF₂, and —OCF₃. In some embodiments, R₅ is —SR_{f}, examples of which include, but are not limited to, —SCH₂F, —SCHF₂, —SCF₃, —SCH₂CH₂F, —SCH₂CHF₂, —SCH₂CF₃, —SCH₂CH₂CH₂F, —SCH₂C H₂CHF₂, —SCH₂CH₂CF₃, —SCH₂CH₂CH₂CH₂F, —SCH₂CH₂CH₂CHF₂, and —SCH₂CH₂CH₂CF₃, with particular mention being made to —SCH₂F, —SCHF₂, —SCF₃.

R₆ and R₇ may be the same, or different. In some embodiments, R₆ and R₇ are the same. In some embodiments, R₆ and R₇ are different. In some embodiments, R₆ is hydrogen. In some embodiments, R₆ is deuterium. In some embodiments, R₆ is a halogen, e.g., fluoro, chloro, bromo, and iodo. In some embodiments, R₆ is an unsubstituted or substituted alkyl (e.g., an unsubstituted or substituted a C₁-C₆ alkyl). In some embodiments, R₆ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₆ is a substituted C₁-C₆ alkyl. When R₆ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₆ is an unsubstituted or substituted alkenyl, e.g., an unsubstituted or substituted allyl. In some embodiments, R₆ is an unsubstituted or substituted alkynyl. In some embodiments, R₆ is an unsubstituted or substituted C₃-C₁₀ cycloalkyl. In some embodiments, R₆ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₆ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₆ is an unsubstituted or substituted heterocycloalkyl. In some embodiments, R₆ is an unsubstituted or substituted aryl. In some embodiments, R₆ is an unsubstituted or substituted heteroaryl.

In some embodiments, R₇ is hydrogen. In some embodiments, R₇ is deuterium. In some embodiments, R₇ is a halogen, e.g., fluoro, chloro, bromo, and iodo. In some embodiments, R₇ is an unsubstituted or substituted alkyl (e.g., an unsubstituted or substituted a C₁-C₆ alkyl). In some embodiments, R₇ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₇ is a substituted C₁-C₆ alkyl. When R₇ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₇ is an unsubstituted or substituted alkenyl, e.g., an unsubstituted or substituted allyl. In some embodiments, R₇ is an unsubstituted or substituted alkynyl. In some embodiments, R₇ is an unsubstituted or substituted C₃-C₁₀ cycloalkyl. In some embodiments, R₇ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₇ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₇ is an unsubstituted or substituted heterocycloalkyl. In some embodiments, R₇ is an unsubstituted or substituted aryl. In some embodiments, R₇ is an unsubstituted or substituted heteroaryl.

R₈ and R₉ may be the same, or different. In some embodiments, R₈ and R₉ are the same. In some embodiments, R₈ and R₇ are different. In some embodiments, R₈ is hydrogen. In some embodiments, R₅ is unsubstituted alkyl. In some embodiments, R₈ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₈ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₈ is R_{f}. Examples of R_{f} include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is unsubstituted alkyl. In some embodiments, R₉ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₉ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₉ is R_{f}, examples of which include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is —S(O) R_{f}, examples of which include, but are not limited to, —S(O) CH₂F, —S(O) CHF₂, —S(O) CF₃, —S(O) CH₂CH₂F, —S(O) CH₂CHF₂, —S(O) CH₂CF₃, —S(O) CH₂CH₂CH₂F, —S(O) CH₂CH₂CHF₂, —S(O) CH₂CH₂CF₃, —S(O) CH₂CH₂CH₂CH₂F, —S(O) CH₂ CH₂CH₂CHF₂, and —S(O) CH₂CH₂CH₂CF₃, with particular mention being made to —S(O) CH₂F, —S(O) CHF₂, and —S(O) CF₃.

In some embodiments, R₉ is —S(O)₂R_{f}, examples of which include, but are not limited to, —S(O)₂CH₂F, —S(O)₂CHF₂, —S(O)₂CF₃, —S(O)₂CH₂CH₂F, —S(O)₂CH₂CHF₂, —S(O)₂CH₂CF₃, —S(O)₂CH₂CH₂CH₂F, —S(O)₂CH₂CH₂CHF₂, —S(O)₂CH₂CH₂CF₃, —S(O)₂CH₂CH₂CH₂CH₂F, —S(O)₂CH₂CH₂CH₂CHF₂, and —S(O)₂CH₂CH₂CH₂CF₃, with particular mention being made to —S(O)₂CH₂F, —S(O)₂CHF₂, and —S(O)₂CF₃.

In some embodiments, R₈ and R₉ together with the nitrogen atom attached thereto are joined to form a heterocycloalkyl substituted with at least one fluorine. In some embodiments, the heterocycloalkyl group may be a 3-membered ring. In some embodiments, the heterocycloalkyl group may be a 4-membered ring. In some embodiments, the heterocycloalkyl group may be a 5-membered ring. In some embodiments, the heterocycloalkyl group may be a 6-membered ring. In some embodiments, the heterocycloalkyl group may be a 7-membered ring. In some embodiments, the heterocycloalkyl group may be an 8-membered ring. The heterocycloalkyl group contains a minimum of one nitrogen ring atom (the nitrogen atom intervening R₈ and R₉), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, with particular mention being made to aziridine, azetidine, pyrrolidine, and piperidine. When R₈ and R₉ together with the nitrogen atom attached thereto are joined to form a heterocycloalkyl group, the heterocycloalkyl group may be substituted with one fluorine atom, two fluorine atoms, three fluorine atoms, four fluorine atoms, or more. In some embodiments, the heterocycloalkyl group is substituted with two fluorine atoms.

Examples of heterocycloalkyl groups substituted with at least one fluorine atom formed from joining R₈ and R₉ together with the nitrogen atom attached thereto include, but are not limited to,

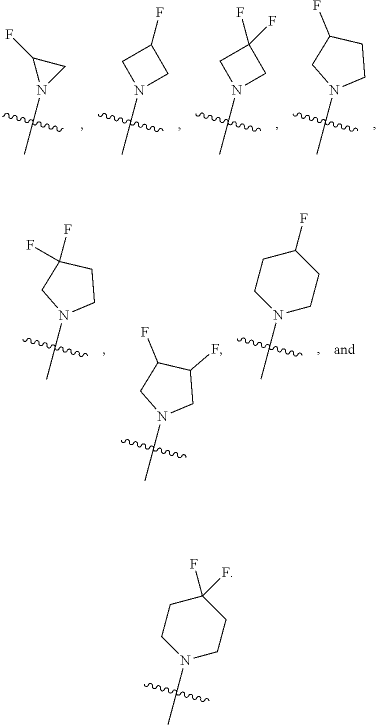

In some embodiments, R₈ and R₉ are not joined with the nitrogen atom attached thereto to form a heterocycloalkyl substituted with at least one fluorine, e.g., those exemplified above. In some embodiments, R₈ and R₉ are not joined with the nitrogen atom attached thereto to form a heterocycloalkyl substituted with at least one fluorine, e.g., those exemplified above, when X₁, X₂, Y₁, Y₂, R₂, R₄, R₅, R₆, and R₇ are hydrogen. In some embodiments, R₈ and R₉ are not joined with the nitrogen atom attached thereto to form a 4,4-difluoropiperidinyl group (represented below) when X₁, X₂, Y₁, Y₂, R₂, R₄, R₅, R₆, and R₇ are hydrogen.

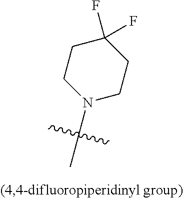 [SMILES: *N1CCC(F)(F)CC1]

In the compounds of the present disclosure, R_{f} represents a fluoroalkyl group, and at least one of R₅, R₈, and Ry comprises the fluoroalkyl group, R_{f}. Each R_{f} present in the disclosed compounds is independently from selected the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium. In some embodiments, each H^{x} is hydrogen. In some embodiments, each H^{x} is deuterium. In some embodiments, at least one H^{x} is deuterium and at least one H^{x} is hydrogen. In some embodiments, n is 0. In some embodiments, n is 1. In some embodiments, n is 2. In some embodiments, n is 3.

Examples of R_{f} include, but are not limited to, —CH₂F, —CHF₂, —CF₃, —CH₂CH₂F, —CH₂CHF₂, —CH₂CF₃, —CD₂CH₂F, —CD₂CHF₂, —CD₂CF₃, —CH₂CH₂CH₂F, —CH₂CH₂CHF₂, —CH₂CH₂CF₃, —CD₂CH₂CH₂F, —CD₂CH₂CHF₂, —CD₂CH₂CF₃, —CD₂CD₂CH₂F, —CD₂CD₂CHF₂, —CD₂CD₂CF₃, —CH₂CH₂CH₂CH₂F, —CH₂CH₂CH₂CHF₂, —CH₂CH₂CH₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, the compound comprises one R_{f} group. In some embodiments, R₅ comprises the fluoroalkyl group, R_{f}, and R₈, and R₉ represent a group that does not comprise the fluoroalkyl group, R_{f}, for example where R₈, and R₉ are each —CH₃ or CD₃, or where R₈ and R₉ together with the nitrogen atom attached thereto are joined to form a heterocycloalkyl substituted with at least one fluorine. In some embodiments, R₉ comprises the fluoroalkyl group, R_{f}, and R₅ and R₅ represent a group that does not comprise the fluoroalkyl group, R_{f}, for example where R₅ is H, —OCH₃, —OCD₃, —SCH₃, —SCD₃, and R₈ is —CH₃ or CD₃.

In some embodiments, two of R₅, R₈, and Ry comprises the fluoroalkyl group, R_{f}, which may be the same or different. In some embodiments, R₈ and R₉ comprise a fluoroalkyl group, R_{f}, which can be the same or different, and R₅ represents a group that does not comprise the fluoroalkyl group, R_{f}, for example where R₅ is H, —OCH₃, —OCD₃, —SCH₃, or —SCD₃. In some embodiments, R₈ and R₉ comprise a fluoroalkyl group, R_{f}, which can be the same or different, and R₅ represents a group that does not comprise the fluoroalkyl group, R_{f}, for example where R₈ is —CH₃ or CD₃.

In some embodiments, each of R₅, R₈, and R₉ comprises the fluoroalkyl group, R_{f}, which may all be the same, all be different, or where two R_{f} groups are the same and the third Rr group is different.

In some embodiments, R₉ is not —CH₂CF₃ when X₁, X₂, Y₁, Y₂, R₂, R₄, R₅, R₆, and R₇ are hydrogen and R₈ is hydrogen or methyl. In some embodiments, R₉ is —CH₂CF₃ when X₁, X₂, Y₁, Y₂, R₂, R₄, R₅, R₆, and R₇ are hydrogen and R₈ is hydrogen or methyl.

In some embodiments, the compound, e.g., the compound of Formula (I), is selected from the group consisting of:

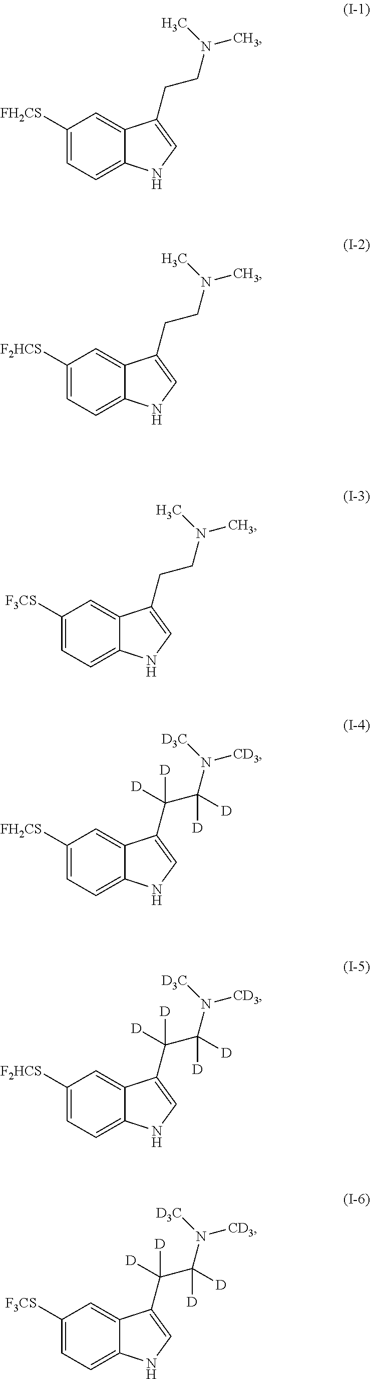

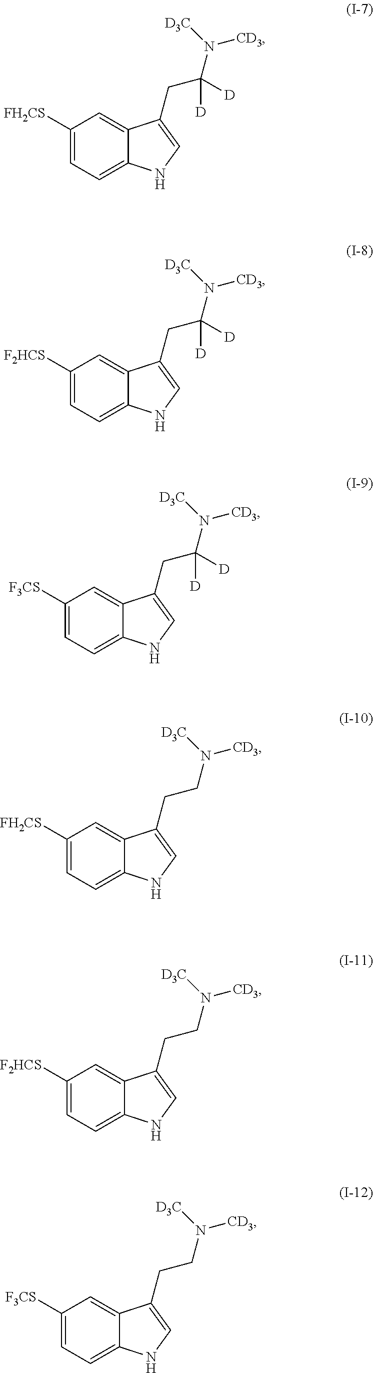

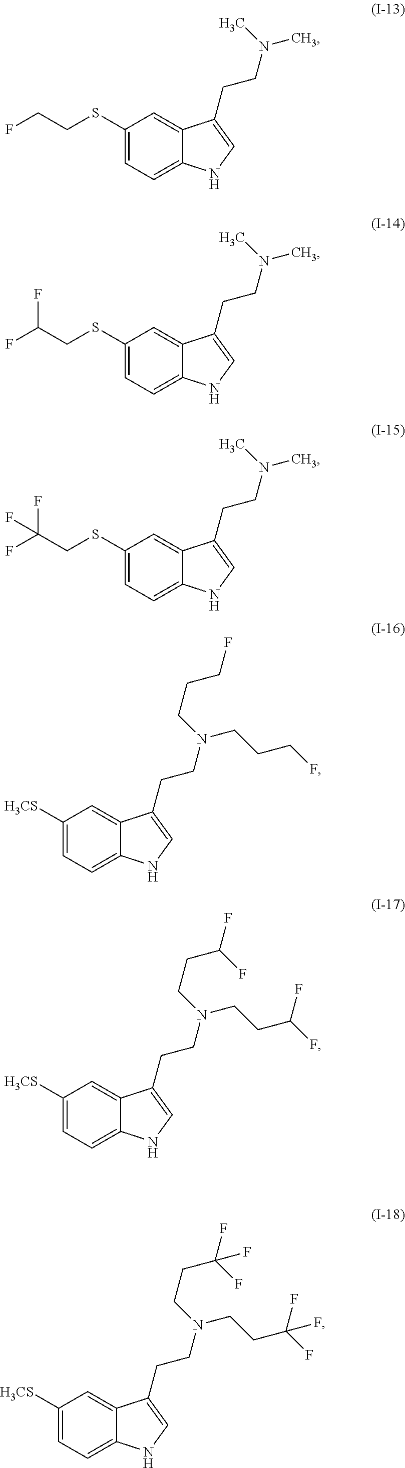

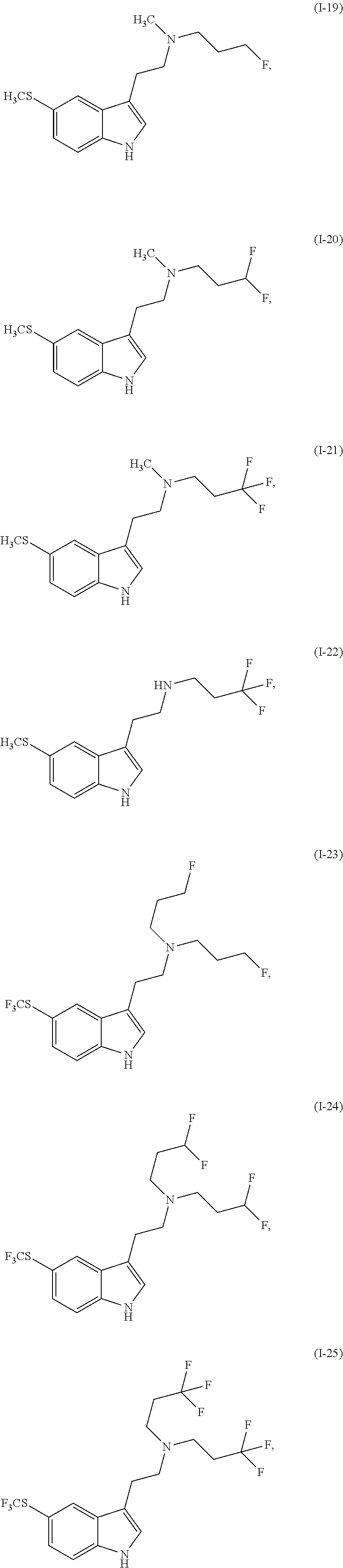

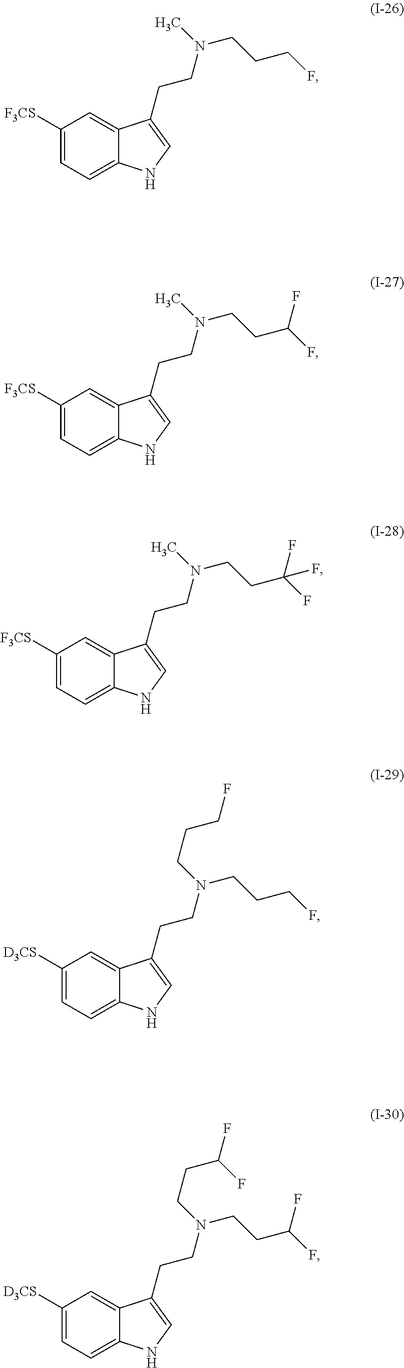

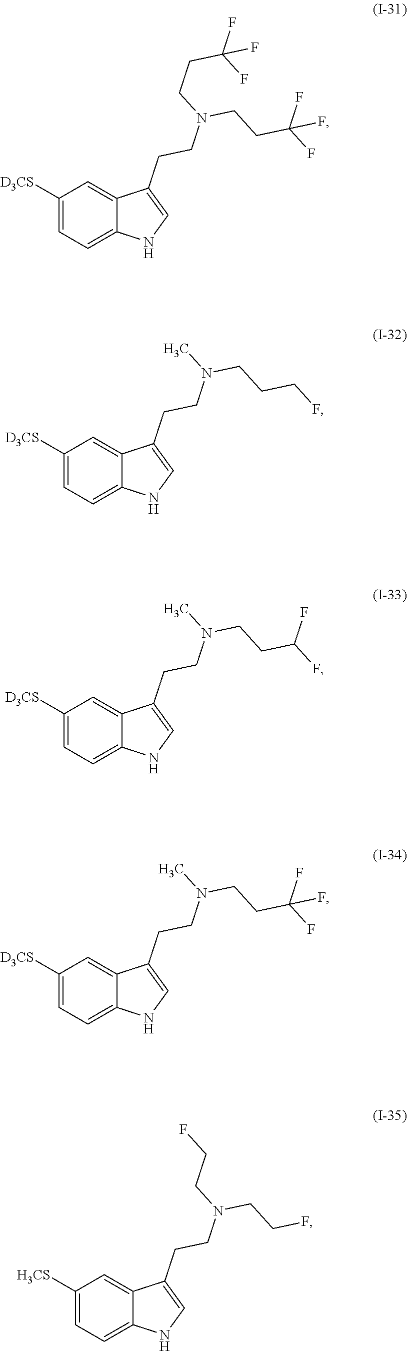

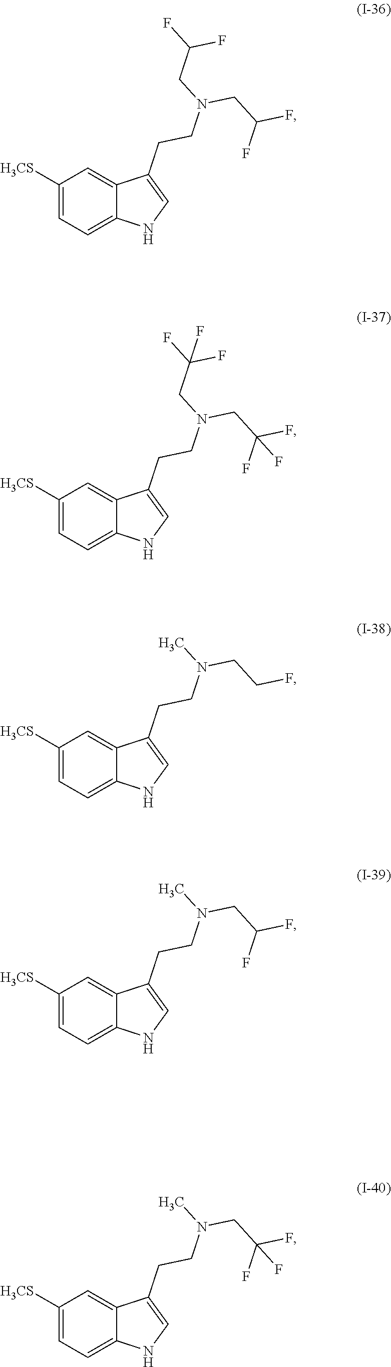

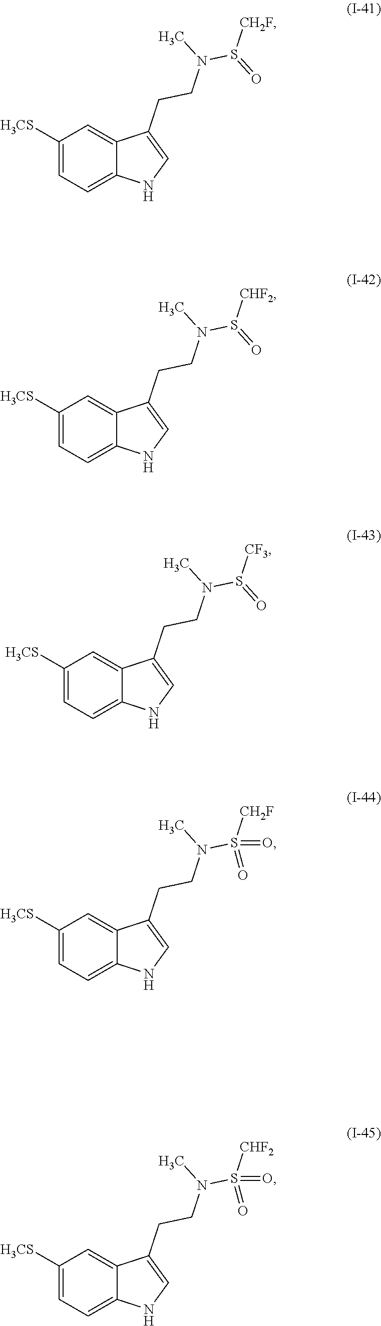

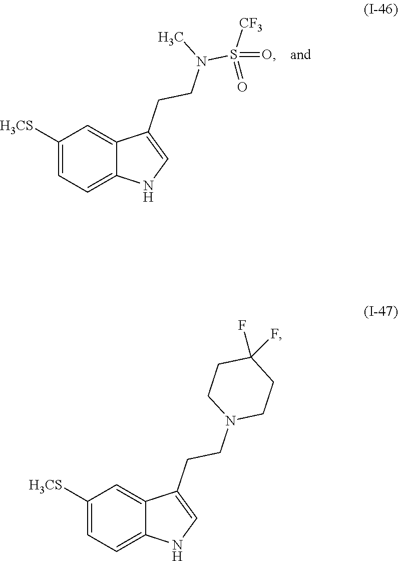

or a pharmaceutically acceptable salt, stereoisomer, tautomer, solvate, polymorph, or prodrug thereof.

In some embodiments, the compound of Formula (I) has a structure of Formula (II), Formula (III), Formula (IV), or Formula (V), including any exemplary compounds thereof, as provided below.

### Formula (II)

In some embodiments, the compound of Formula (I) has a structure of Formula (II), or a pharmaceutically acceptable salt, stereoisomer, tautomer, solvate, polymorph, or prodrug thereof,

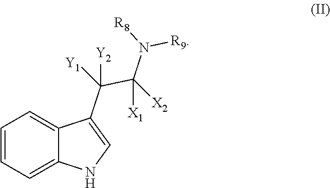

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f};
or alternatively R₈ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₈ and R₉ comprises the fluoroalkyl group, R_{f}, or R₈ and R₉ together with the nitrogen atom attached thereto are joined to form the heterocycloalkyl substituted with at least one fluorine.
X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is an unsubstituted or substituted alkyl (e.g., an unsubstituted or substituted a C₁-C₆ alkyl). In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, X₁ and/or X₂ is an unsubstituted or substituted alkenyl, e.g., an unsubstituted or substituted allyl. In some embodiments, X₁ and/or X₂ is an unsubstituted or substituted alkynyl. In some embodiments, X₁ and/or X₂ is an unsubstituted or substituted C₃-C₁₀ cycloalkyl. In some embodiments, X₁ and/or X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, X₁ and/or X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent.

In some embodiments, X₁ and/or X₂ is an unsubstituted or substituted heterocycloalkyl. In some embodiments, X₁ and/or X₂ is an unsubstituted or substituted aryl. In some embodiments, X₁ and/or X₂ is an unsubstituted or substituted heteroaryl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

R₈ and R₉ may be the same, or different. In some embodiments, R₈ and R₉ are the same. In some embodiments, R₈ and R₈ are different. In some embodiments, Rₐ is hydrogen. In some embodiments, R₈ is unsubstituted alkyl. In some embodiments, R₈ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₈ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, Rₐ is R_{f}. Examples of R_{f} include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH 2CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is unsubstituted alkyl. In some embodiments, R₉ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₉ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₉ is R_{f}, examples of which include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is —S(O) R_{f}, examples of which include, but are not limited to, —S(O) CH₂F, —S(O) CHF₂, —S(O) CF₃, —S(O) CH₂CH₂F, —S(O) CH₂CHF₂, —S(O) CH₂CF₃, —S(O) CH₂CH₂CH₂F, —S(O) CH₂CH₂CHF₂, —S(O) CH₂CH₂CF₃, —S(O) CH₂CH₂CH₂CH₂F, —S(O) CH₂ CH₂CH₂CHF₂, and —S(O) CH₂CH₂CH₂CF₃, with particular mention being made to —S(O) CH₂F, —S(O) CHF₂, and —S(O) CF₃.

In some embodiments, R₉ is —S(O)₂R_{f}, examples of which include, but are not limited to, —S(O)₂CH₂F, —S(O)₂CHF₂, —S(O)₂CF₃, —S(O)₂CH₂CH₂F, —S(O)₂CH₂CHF₂, —S(O)₂CH₂CF₃, —S(O)₂CH₂CH₂CH₂F, —S(O)₂CH₂CH₂CHF₂, —S(O)₂CH₂CH₂CF₃, —S(O)₂CH₂CH₂CH₂CH₂F, —S(O)₂CH₂CH₂CH₂CHF₂, and —S(O)₂CH₂CH₂CH₂CF₃, with particular mention being made to —S(O)₂CH₂F, —S(O)₂CHF₂, and —S(O)₂CF₃.

In some embodiments, R₈ and R₉ together with the nitrogen atom attached thereto are joined to form a heterocycloalkyl substituted with at least one fluorine. In some embodiments, the heterocycloalkyl group may be a 3-membered ring. In some embodiments, the heterocycloalkyl group may be a 4-membered ring. In some embodiments, the heterocycloalkyl group may be a 5-membered ring. In some embodiments, the heterocycloalkyl group may be a 6-membered ring. In some embodiments, the heterocycloalkyl group may be a 7-membered ring. In some embodiments, the heterocycloalkyl group may be an 8-membered ring. The heterocycloalkyl group contains a minimum of one nitrogen ring atom (the nitrogen atom intervening R₈ and R₉), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, with particular mention being made to aziridine, azetidine, pyrrolidine, and piperidine. When R₈ and R₉ together with the nitrogen atom attached thereto are joined to form a heterocycloalkyl group, the heterocycloalkyl group may be substituted with one fluorine atom, two fluorine atoms, three fluorine atoms, four fluorine atoms, or more. In some embodiments, the heterocycloalkyl group is substituted with two fluorine atoms.

Examples of heterocycloalkyl groups substituted with at least one fluorine atom formed from joining R₈ and R₉ together with the nitrogen atom attached thereto include, but are not limited to,

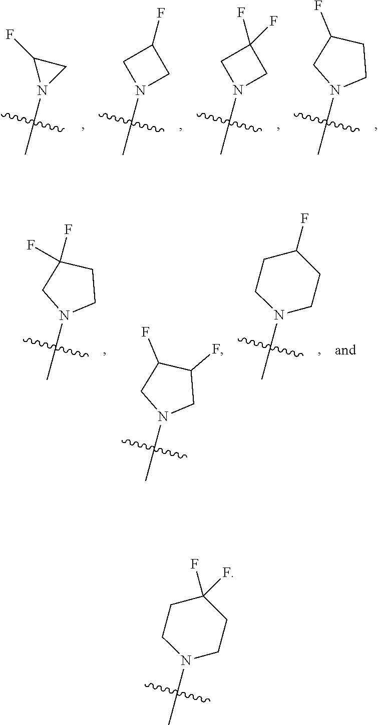

In some embodiments, R₈ and R₉ are not joined with the nitrogen atom attached thereto to form a heterocycloalkyl substituted with at least one fluorine, e.g., those exemplified above. In some embodiments, R₈ and R₉ are not joined with the nitrogen atom attached thereto to form a heterocycloalkyl substituted with at least one fluorine, e.g., those exemplified above, when X₁, X₂, Y₁, and Y₂ are hydrogen. In some embodiments, R₈ and R₉ are not joined with the nitrogen atom attached thereto to form a 4,4-difluoropiperidinyl group (represented below) when X₁, X₂, Y₁, and Y₂ are hydrogen.

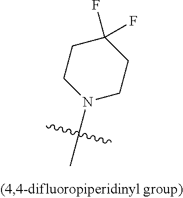 [SMILES: *N1CCC(F)(F)CC1]

R_{f} represents a fluoroalkyl group. In the compound of Formula (II), at least one of R₈ and R₉ comprises the fluoroalkyl group, R_{f}. Each R_{f} present in the disclosed compounds is independently selected from the group consisting of —(CH^{x}₂)ₙCH₂F, —(CH^{x}₂)ₙCHF₂, and —(CH^{x}₂)ₙCF₃, wherein n is 0 to 3, and each Ha is independently hydrogen or deuterium. In some embodiments, each H^{x} is hydrogen. In some embodiments, each H is deuterium. In some embodiments, at least one H^{x} is deuterium and at least one H^{x} is hydrogen. In some embodiments, n is 0. In some embodiments, n is 1. In some embodiments, n is 2. In some embodiments, n is 3.

Examples of R_{f} include, but are not limited to, —CH₂F, —CHF₂, —CF₃, —CH₂CH₂F, —CH₂CHF₂, —CH₂CF₃, —CD₂CH₂F, —CD₂CHF₂, —CD₂CF₃, —CH₂CH₂CH₂F, —CH₂CH₂CHF₂, —CH₂CH₂CF₃, —CD₂CH₂CH₂F, —CD₂CH₂CHF₂, —CD₂CH₂CF₃, —CD₂CD₂CH₂F, —CD₂CD₂CHF₂, —CD₂CD₂CF₃, —CH₂CH₂CH₂CH₂F, —CH₂CH₂CH₂CHF₂, —CH₂CH₂CH₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃

In some embodiments, the compound comprises one R_{f} group. In some embodiments, R⁹ comprises the fluoroalkyl group, R_{f}, and R₅ represents a group that does not comprise the fluoroalkyl group, R_{f}, for example where R₅ is —CH₃ or CD₃. In some embodiments, both R₈ and R₉ comprise the fluoroalkyl group, R_{f}, which may be the same or different.

In some embodiments, R₉ is not —CH₂CF₃ when X₁, X₂, Y₁, and Y₂ are hydrogen and R₈ is hydrogen or methyl.

In some embodiments, the compound, e.g., the compound of Formula (II), is selected from the group consisting of:

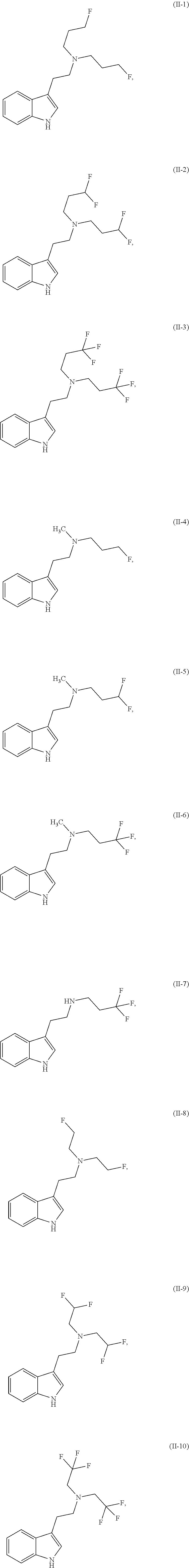

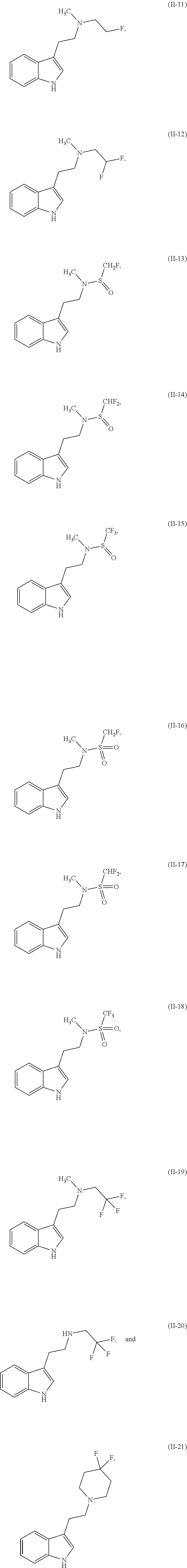

or a pharmaceutically acceptable salt, stereoisomer, tautomer, solvate, polymorph, or prodrug thereof.

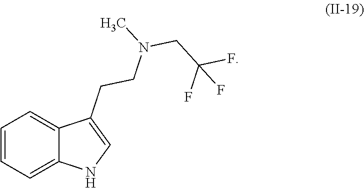 [SMILES: CN(CCc1c[nH]c2ccccc12)CC(F)(F)F]

In some embodiments, the compound is not

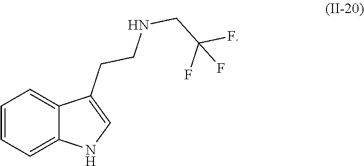 [SMILES: FC(F)(F)CNCCc1c[nH]c2ccccc12]

In some embodiments, the compound is not

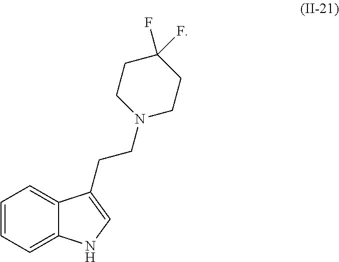 [SMILES: FC1(F)CCN(CCc2c[nH]c3ccccc23)CC1]

In some embodiments, the compound is not

### Formula (III)

In some embodiments, the compound of Formula (I) has a structure of Formula (III), or a pharmaceutically acceptable salt, stereoisomer, tautomer, solvate, polymorph, or prodrug thereof,

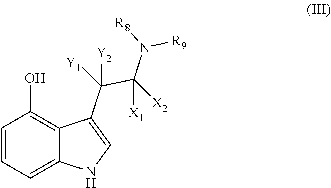

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f};
or alternatively R₈ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₈ and R₉ comprises the fluoroalkyl group, R_{f}, or R₈ and R₉ together with the nitrogen atom attached thereto are joined to form the heterocycloalkyl substituted with at least one fluorine.
X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is an unsubstituted or substituted alkyl (e.g., an unsubstituted or substituted a C₁-C₆ alkyl). In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, X₁ and/or X₂ is an unsubstituted or substituted alkenyl, e.g., an unsubstituted or substituted allyl. In some embodiments, X₁ and/or X₂ is an unsubstituted or substituted alkynyl. In some embodiments, X₁ and/or X₂ is an unsubstituted or substituted C₃-C₁₀ cycloalkyl. In some embodiments, X₁ and/or X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, X₁ and/or X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent.

In some embodiments, X₁ and/or X₂ is an unsubstituted or substituted heterocycloalkyl. In some embodiments, X₁ and/or X₂ is an unsubstituted or substituted aryl. In some embodiments, X₁ and/or X₂ is an unsubstituted or substituted heteroaryl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

R₈ and R₉ may be the same, or different. In some embodiments, R₈ and R₉ are the same. In some embodiments, R₈ and R₉ are different. In some embodiments, Rₐ is hydrogen. In some embodiments, R₈ is unsubstituted alkyl. In some embodiments, R₅ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₈ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₈ is R_{f}. Examples of R_{f} include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is unsubstituted alkyl. In some embodiments, R₉ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₉ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₉ is R_{f}, examples of which include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is —S(O) R_{f}, examples of which include, but are not limited to, —S(O) CH₂F, —S(O) CHF₂, —S(O) CF₃, —S(O) CH₂CH₂F, —S(O) CH₂CHF₂, —S(O) CH₂CF₃, —S(O) CH₂CH₂CH₂F, —S(O) CH₂CH₂CHF₂, —S(O) CH₂CH₂CF₃, —S(O) CH₂CH₂CH₂CH₂F, —S(O) CH₂ CH₂CH₂CHF₂, and —S(O) CH₂CH₂CH₂CF₃, with particular mention being made to —S(O) CH₂F, —S(O) CHF₂, and —S(O) CF₃.

In some embodiments, R₉ is —S(O)₂R_{f}, examples of which include, but are not limited to, —S(O)₂CH₂F, —S(O)₂CHF₂, —S(O)₂CF₃, —S(O)₂CH₂CH₂F, —S(O)₂CH₂CHF₂, —S(O)₂CH₂CF₃, —S(O)₂CH₂CH₂CH₂F, —S(O)₂CH₂CH₂CHF₂, —S(O)₂CH₂CH₂CF₃, —S(O)₂CH₂CH₂CH₂CH₂F, —S(O)₂CH₂CH₂CH₂CHF₂, and —S(O)₂CH₂CH₂CH₂CF₃, with particular mention being made to —S(O)₂CH₂F, —S(O)₂CHF₂, and —S(O)₂CF₃.

In some embodiments, R₈ and R₉ together with the nitrogen atom attached thereto are joined to form a heterocycloalkyl substituted with at least one fluorine. In some embodiments, the heterocycloalkyl group may be a 3-membered ring. In some embodiments, the heterocycloalkyl group may be a 4-membered ring. In some embodiments, the heterocycloalkyl group may be a 5-membered ring. In some embodiments, the heterocycloalkyl group may be a 6-membered ring. In some embodiments, the heterocycloalkyl group may be a 7-membered ring. In some embodiments, the heterocycloalkyl group may be an 8-membered ring. The heterocycloalkyl group contains a minimum of one nitrogen ring atom (the nitrogen atom intervening R₈ and R₉), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, with particular mention being made to aziridine, azetidine, pyrrolidine, and piperidine. When R₅ and R₉ together with the nitrogen atom attached thereto are joined to form a heterocycloalkyl group, the heterocycloalkyl group may be substituted with one fluorine atom, two fluorine atoms, three fluorine atoms, four fluorine atoms, or more. In some embodiments, the heterocycloalkyl group is substituted with two fluorine atoms.

Examples of heterocycloalkyl groups substituted with at least one fluorine atom formed from joining R₈ and R₉ together with the nitrogen atom attached thereto include, but are not limited to,

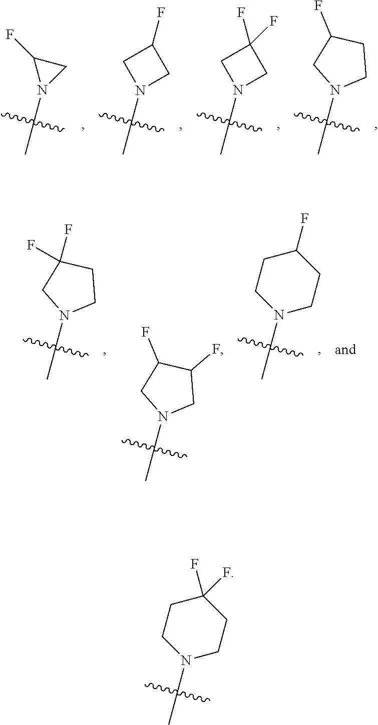

In some embodiments, R₈ and R₉ are not joined with the nitrogen atom attached thereto to form a heterocycloalkyl substituted with at least one fluorine, e.g., those exemplified above. In some embodiments, R₈ and R₇ are not joined with the nitrogen atom attached thereto to form a heterocycloalkyl substituted with at least one fluorine, e.g., those exemplified above, when X₁, X₂, Y₁, and Y₂ are hydrogen. In some embodiments, R₈ and R₉ are not joined with the nitrogen atom attached thereto to form a 4,4-difluoropiperidinyl group (represented below) when X₁, X₂, Y₁, and Y₂ are hydrogen.

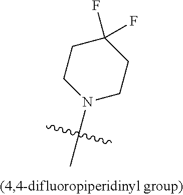 [SMILES: *N1CCC(F)(F)CC1]

R_{f} represents a fluoroalkyl group. In the compound of Formula (III), at least one of R₈ and R₉ comprises the fluoroalkyl group, R_{f}. Each R_{f} present in the disclosed compounds is independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium. In some embodiments, each H^{x} is hydrogen. In some embodiments, each H^{x} is deuterium. In some embodiments, at least one H is deuterium and at least one H^{x} is hydrogen. In some embodiments, n is 0. In some embodiments, n is 1. In some embodiments, n is 2. In some embodiments, n is 3.

Examples of R_{f} include, but are not limited to, —CH₂F, —CHF₂, —CF₃, —CH₂CH₂F, —CH₂CHF₂, —CH₂CF₃, —CD₂CH₂F, —CD₂CHF₂, —CD₂CF₃, —CH₂CH₂CH₂F, —CH₂CH₂CHF₂, —CH₂CH₂CF₃, —CD₂CH₂CH₂F, —CD₂CH₂CHF₂, —CD₂CH₂CF₃, —CD₂CD₂CH₂F, —CD₂CD₂CHF₂, —CD₂CD₂CF₃, —CH₂CH₂CH₂CH₂F, —CH₂CH₂CH₂CHF₂, —CH₂CH₂CH₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, the compound comprises one R_{f} group. In some embodiments, R₉ comprises the fluoroalkyl group, R_{f}, and R₈ represents a group that does not comprise the fluoroalkyl group, R_{f}, for example where R₈ is —CH₃ or CD₃. In some embodiments, both R₈ and R₉ comprise the fluoroalkyl group, R_{f}, which may be the same or different.

In some embodiments, the compound, e.g., the compound of Formula (III), is selected from the group consisting of:

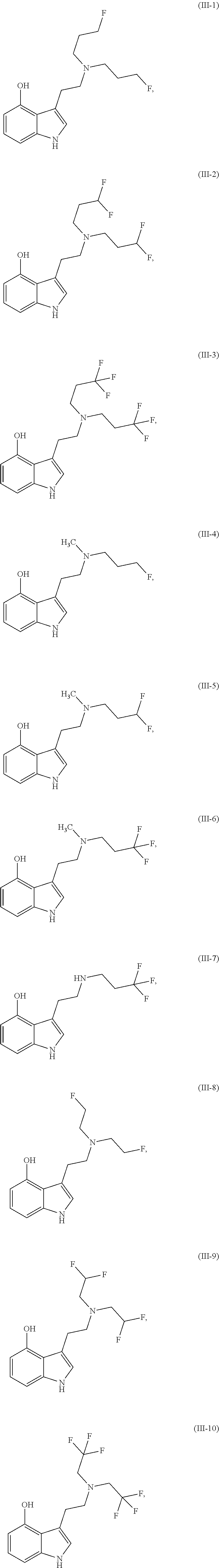

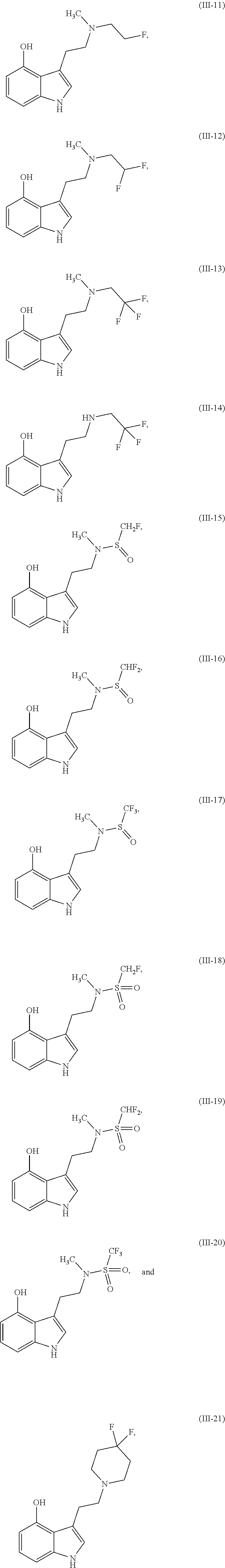

or a pharmaceutically acceptable salt, stereoisomer, tautomer, solvate, polymorph, or prodrug thereof.

### Formula (IV)

In some embodiments, the compound of Formula (I) has a structure of Formula (IV), or a pharmaceutically acceptable salt, stereoisomer, tautomer, solvate, polymorph, or prodrug thereof,

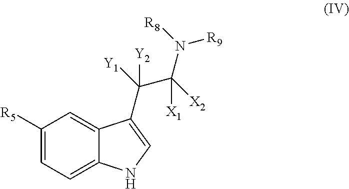

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of unsubstituted alkoxy, alkoxy substituted with one or more deuterium, and —OR_{f};
R₈ is selected from the group consisting of hydrogen, unsubstituted alkyl, an alkyl substituted with one or more deuterium, and R_{f},
R₉ is selected from the group consisting of unsubstituted alkyl, an alkyl substituted with one or more deuterium, R_{f}, —S(O) R_{f}, and —S(O)₂R_{f}, or alternatively R₈ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine.
X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is an unsubstituted or substituted alkyl (e.g., an unsubstituted or substituted a C₁-C₆ alkyl). In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, X₁ and/or X₂ is an unsubstituted or substituted alkenyl, e.g., an unsubstituted or substituted allyl. In some embodiments, X₁ and/or X₂ is an unsubstituted or substituted alkynyl. In some embodiments, X₁ and/or X₂ is an unsubstituted or substituted C₃-C₁₀ cycloalkyl. In some embodiments, X₁ and/or X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, X₁ and/or X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, X₁ and/or X₂ is an unsubstituted or substituted heterocycloalkyl. In some embodiments, X₁ and/or X₂ is an unsubstituted or substituted aryl. In some embodiments, X₁ and/or X₂ is an unsubstituted or substituted heteroaryl.
Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₅ is an unsubstituted alkoxy group, such as an unsubstituted C₁-C₆ alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is an alkoxy group substituted with one or more deuterium.

The alkoxy group may contain one, or more than one, deuterium substituent. For example, when the alkoxy group is a C₁ alkoxy group (i.e., methoxy group), the deuterium substituted C₁ alkoxy group may be —OCDH₂, —OCD₂H, and —OCD₃.

In some embodiments, R₅ is —OR_{f}, examples of which include, but are not limited to, —OCH₂F, —OCHF₂, —OCF₃, —OCH₂CH₂F, —OCH₂CHF₂, —OCH₂CF₃, —OCH₂CH₂CH₂F, —OCH₂CH₂CHF₂, —OCH₂CH₂CF₃, —OCH₂CH₂CH₂CH₂F, —OCH₂CH₂CH₂CHF₂, and —OCH₂CH₂CH₂CF₃, with particular mention being made to —OCH₂F, —OCHF₂, and —OCF₃.

R₈ and R₉ may be the same, or different. In some embodiments, R₈ and R₇ are the same. In some embodiments, R₈ and R₉ are different. In some embodiments, R₈ is hydrogen. In some embodiments, R₈ is unsubstituted alkyl. In some embodiments, R₈ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₈ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₈ is R_{f}. Examples of R_{f} include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is unsubstituted alkyl. In some embodiments, R₉ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₉ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₉ is R_{f}, examples of which include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is —S(O) R_{f}, examples of which include, but are not limited to, —S(O) CH₂F, —S(O) CHF₂, —S(O) CF₃, —S(O) CH₂CH₂F, —S(O) CH₂CHF₂, —S(O) CH₂CF₃, —S(O) CH₂CH₂CH₂F, —S(O) CH₂CH₂CHF₂, —S(O) CH₂CH₂CF₃, —S(O) CH₂CH₂CH₂CH₂F, —S(O) CH₂ CH₂CH₂CHF₂, and —S(O) CH₂CH₂CH₂CF₃, with particular mention being made to —S(O) CH₂F, —S(O) CHF₂, and —S(O) CF₃.

In some embodiments, R₉ is —S(O)₂R_{f}, examples of which include, but are not limited to, —S(O)₂CH₂F, —S(O)₂CHF₂, —S(O)₂CF₃, —S(O)₂CH₂CH₂F, —S(O)₂CH₂CHF₂, —S(O)₂CH₂CF₃, —S(O)₂CH₂CH₂CH₂F, —S(O)₂CH₂CH₂CHF₂, —S(O)₂CH₂CH₂CF₃, —S(O)₂CH₂CH₂CH₂CH₂F, —S(O)₂CH₂CH₂CH₂CHF₂, and —S(O)₂CH₂CH₂CH₂CF₃, with particular mention being made to —S(O)₂CH₂F, —S(O)₂CHF₂, and —S(O)₂CF₃.

In some embodiments, R₈ and R₉ together with the nitrogen atom attached thereto are joined to form a heterocycloalkyl substituted with at least one fluorine. In some embodiments, the heterocycloalkyl group may be a 3-membered ring. In some embodiments, the heterocycloalkyl group may be a 4-membered ring. In some embodiments, the heterocycloalkyl group may be a 5-membered ring. In some embodiments, the heterocycloalkyl group may be a 6-membered ring. In some embodiments, the heterocycloalkyl group may be a 7-membered ring. In some embodiments, the heterocycloalkyl group may be an 8-membered ring. The heterocycloalkyl group contains a minimum of one nitrogen ring atom (the nitrogen atom intervening R₈ and R₉), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, with particular mention being made to aziridine, azetidine, pyrrolidine, and piperidine. When R₈ and R₉ together with the nitrogen atom attached thereto are joined to form a heterocycloalkyl group, the heterocycloalkyl group may be substituted with one fluorine atom, two fluorine atoms, three fluorine atoms, four fluorine atoms, or more. In some embodiments, the heterocycloalkyl group is substituted with two fluorine atoms.

Examples of heterocycloalkyl groups substituted with at least one fluorine atom formed from joining R₈ and R₉ together with the nitrogen atom attached thereto include, but are not limited to,

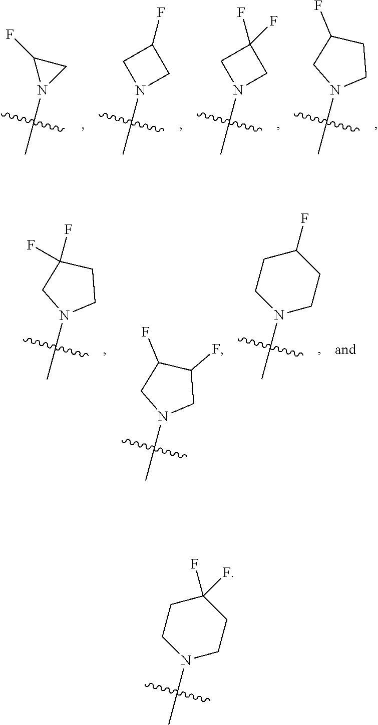

In some embodiments, R₈ and R₉ are not joined with the nitrogen atom attached thereto to form a heterocycloalkyl substituted with at least one fluorine, e.g., those exemplified above. In some embodiments, R₈ and R₉ are not joined with the nitrogen atom attached thereto to form a heterocycloalkyl substituted with at least one fluorine, e.g., those exemplified above, when X₁, X₂, Y₁, and Y₂ are hydrogen. In some embodiments, R₈ and R₉ are not joined with the nitrogen atom attached thereto to form a 4,4-difluoropiperidinyl group (represented below) when X₁, X₂, Y₁, and Y₂ are hydrogen.

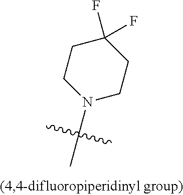 [SMILES: *N1CCC(F)(F)CC1]

R_{f} represents a fluoroalkyl group. In the compound of Formula (IV), at least one of R₅, R₈, and R₉ comprises the fluoroalkyl group, R_{f}. Each R_{f} present in the disclosed compounds is independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium. In some embodiments, each H^{x} is hydrogen. In some embodiments, each H^{x} is deuterium. In some embodiments, at least one H^{x} is deuterium and at least one H^{x} is hydrogen. In some embodiments, n is 0. In some embodiments, n is 1. In some embodiments, n is 2. In some embodiments, n is 3.

Examples of R_{f} include, but are not limited to, —CH₂F, —CHF₂, —CF₃, —CH₂CH₂F, —CH₂CHF₂, —CH₂CF₃, —CD₂CH₂F, —CD₂CHF₂, —CD₂CF₃, —CH₂CH₂CH₂F, —CH₂CH₂CHF₂, —CH₂CH₂CF₃, —CD₂CH₂CH₂F, —CD₂CH₂CHF₂, —CD₂CH₂CF₃, —CD₂CD₂CH₂F, —CD₂CD₂CHF₂, —CD₂CD₂CF₃, —CH₂CH₂CH₂CH₂F, —CH₂CH₂CH₂CHF₂, —CH₂CH₂CH₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, the compound comprises one R_{f} group. In some embodiments, R₅ comprises the fluoroalkyl group, R_{f}, and R₈, and R₉ represent a group that does not comprise the fluoroalkyl group, R_{f}, for example where R₅, and R₉ are each —CH₃ or CD₃. In some embodiments, R₉ comprises the fluoroalkyl group, R_{f}, and R₅ and R₅ represent a group that does not comprise the fluoroalkyl group, R_{f}, for example where R₅ is —OCH₃ or —OCD₃ and R₈ is —CH₃ or CD₃.

In some embodiments, two of R₅, R₈, and Ry comprises the fluoroalkyl group, R_{f}, which may be the same or different. In some embodiments, R₈ and R comprise a fluoroalkyl group,

R_{f}, which can be the same or different, and R₅ represents a group that does not comprise the fluoroalkyl group, R_{f}, for example where R₅—OCH₃ or —OCD₃. In some embodiments, R₅ and R₉ comprise a fluoroalkyl group, R_{f}, which can be the same or different, and R₈ represents a group that does not comprise the fluoroalkyl group, R_{f}, for example where R₅ is —CH₃ or CD₃.

In some embodiments, each of R₅, R₈, and R₉ comprises the fluoroalkyl group, R_{f}, which may all be the same, all be different, or where two R_{f} groups are the same and the third R_{f} group is different.

In some embodiments, the compound, e.g., the compound of Formula (IV), is selected from the group consisting of:

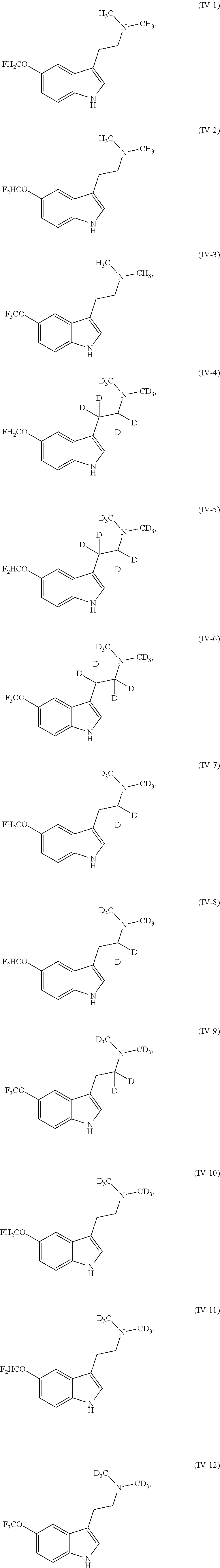

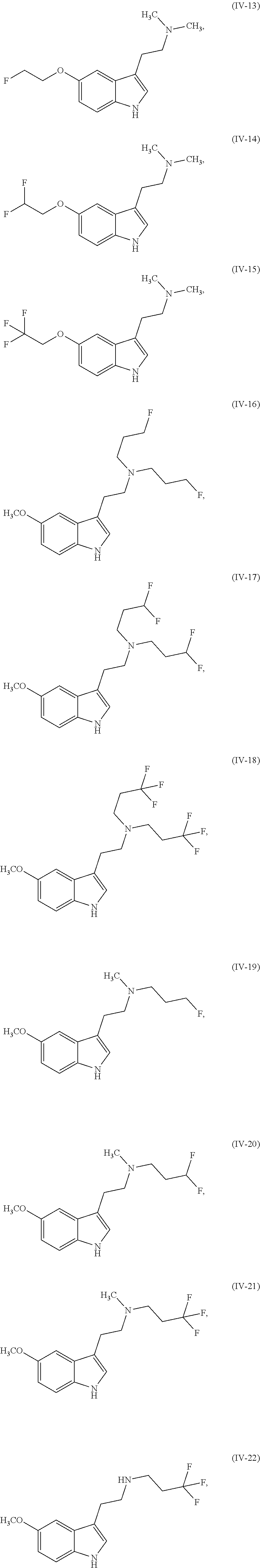

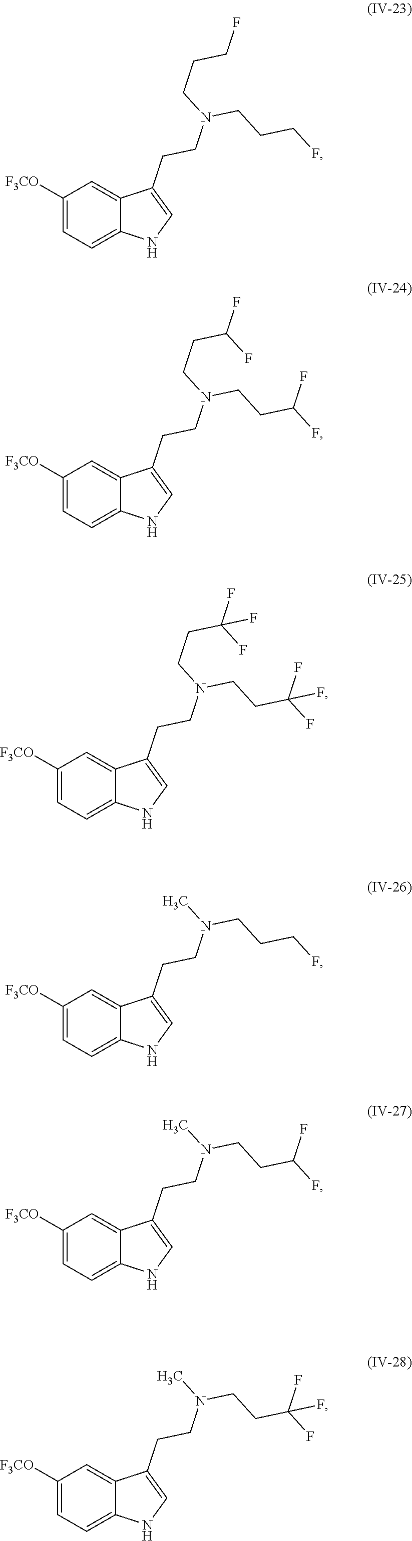 [SMILES: FCCCN(CCCF)CCc1c[nH]c2ccc(OC(F)(F)F)cc12 ; FC(F)CCN(CCc1c[nH]c2ccc(OC(F)(F)F)cc12)CCC(F)F ; FC(F)(F)CCN(CCc1c[nH]c2ccc(OC(F)(F)F)cc12)CCC(F)(F)F ; CN(CCCF)CCc1c[nH]c2ccc(OC(F)(F)F)cc12 ; CN(CCc1c[nH]c2ccc(OC(F)(F)F)cc12)CCC(F)F ; CN(CCc1c[nH]c2ccc(OC(F)(F)F)cc12)CCC(F)(F)F]

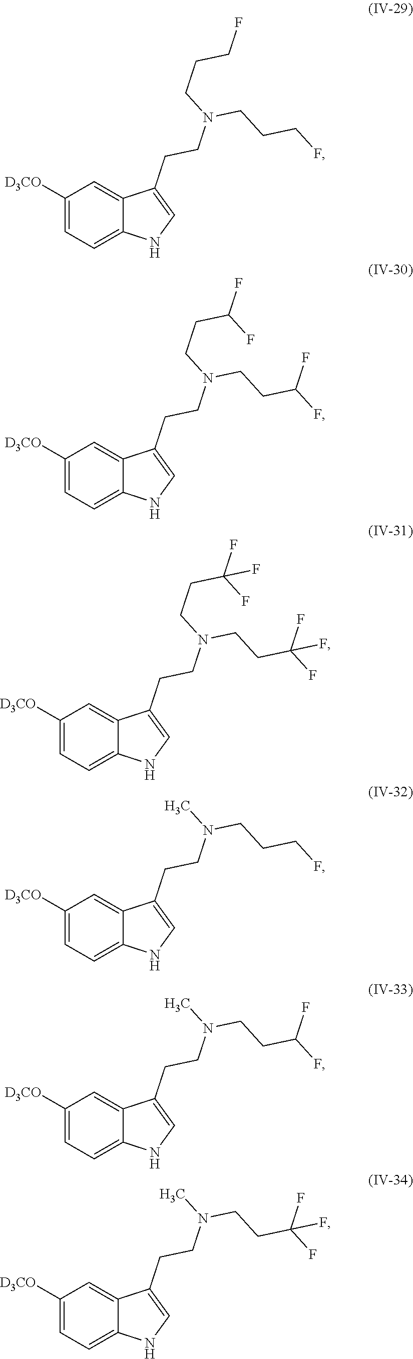

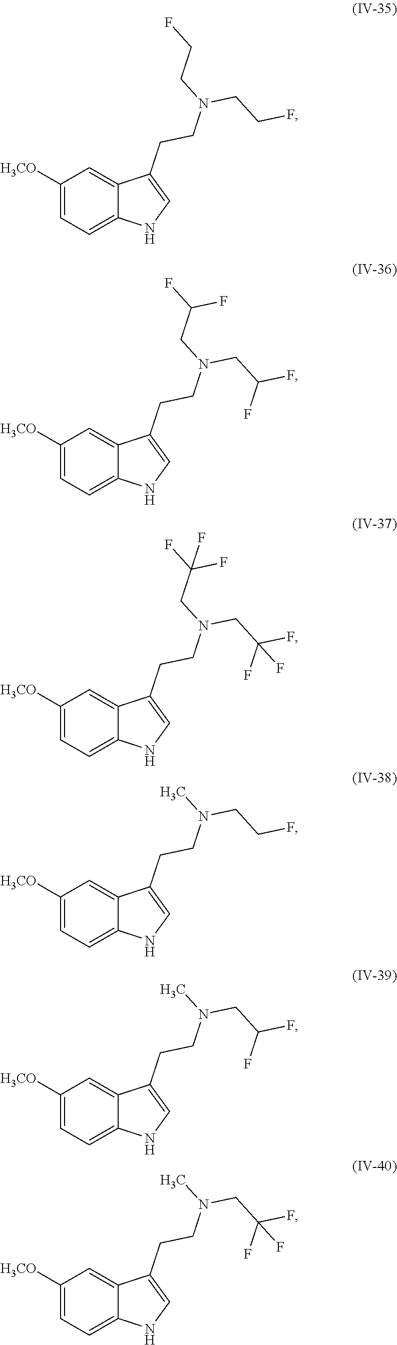 [SMILES: COc1ccc2[nH]cc(CCN(CCF)CCF)c2c1 ; COc1ccc2[nH]cc(CCN(CC(F)F)CC(F)F)c2c1 ; COc1ccc2[nH]cc(CCN(CC(F)(F)F)CC(F)(F)F)c2c1 ; COc1ccc2[nH]cc(CCN(C)CCF)c2c1 ; COc1ccc2[nH]cc(CCN(C)CC(F)F)c2c1 ; COc1ccc2[nH]cc(CCN(C)CC(F)(F)F)c2c1]

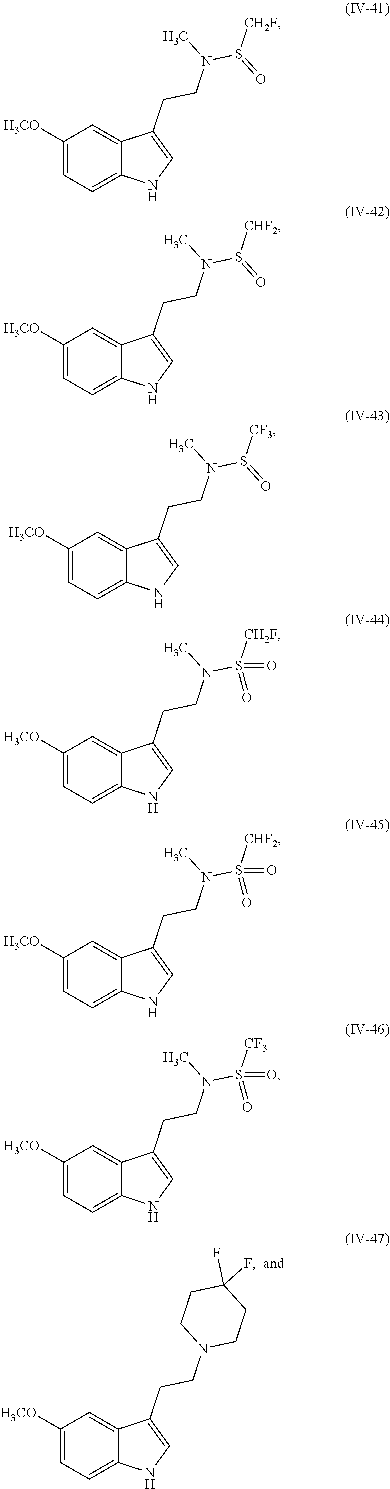

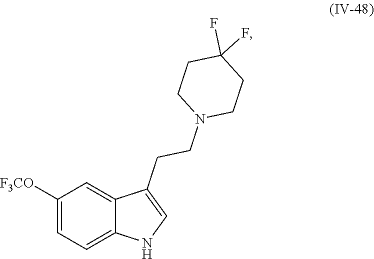 [SMILES: FC1(F)CCN(CCc2c[nH]c3ccc(OC(F)(F)F)cc23)CC1]

or a pharmaceutically acceptable salt, stereoisomer, tautomer, solvate, polymorph, or prodrug thereof.

### Formula (V)

In some embodiments, the compound of Formula (I) has a structure of Formula (V), or a pharmaceutically acceptable salt, stereoisomer, tautomer, solvate, polymorph, or prodrug thereof,

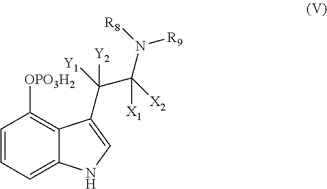

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f}, or alternatively R₈ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of Rₐ and Ry comprises the fluoroalkyl group, R_{f}, or R₈ and R₉ together with the nitrogen atom attached thereto are joined to form the heterocycloalkyl substituted with at least one fluorine.
X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is an unsubstituted or substituted alkyl (e.g., an unsubstituted or substituted a C₁-C₆ alkyl). In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, X₁ and/or X₂ is an unsubstituted or substituted alkenyl, e.g., an unsubstituted or substituted allyl. In some embodiments, X₁ and/or X₂ is an unsubstituted or substituted alkynyl. In some embodiments, X₁ and/or X₂ is an unsubstituted or substituted C₃-C₁₀ cycloalkyl. In some embodiments, X₁ and/or X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, X₁ and/or X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent.

In some embodiments, X₁ and/or X₂ is an unsubstituted or substituted heterocycloalkyl. In some embodiments, X₁ and/or X₂ is an unsubstituted or substituted aryl. In some embodiments, X₁ and/or X₂ is an unsubstituted or substituted heteroaryl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

R₈ and R₉ may be the same, or different. In some embodiments, R₈ and R₉ are the same. In some embodiments, R₈ and R₉ are different. In some embodiments, R₈ is hydrogen. In some embodiments, R₈ is unsubstituted alkyl. In some embodiments, Rₐ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, R₈ is an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₈ is R_{f}. Examples of R_{f} include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH 2CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is unsubstituted alkyl. In some embodiments, R₉ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl, with particular mention being made to methyl. In some embodiments, Rois an alkyl substituted with one or more deuterium, e.g., a C₁-C₆ alkyl group substituted with one or more deuterium. The alkyl group may contain one, or more than one, deuterium substituent. For example, when the alkyl group is a C₁ alkyl group (i.e., methyl group), the deuterium substituted C₁ alkyl group may be —CDH₂, —CD₂H, and —CD₃, with particular mention being made to —CD₃.

In some embodiments, R₉ is R_{f}, examples of which include, but are not limited to, —CH₂CH₂F, —CH₂CHF₂, —CH₂CF₃, —CH₂CH₂CH₂F, —CH₂CH₂CHF₂, —CH₂CH₂CF₃, —CH₂CH 2CH₂CH₂F, —CH₂CH₂CH₂CHF₂, and —CH₂CH₂CH₂CF₃, with particular mention being made to —CH₂CH₂CH₂F, —CH₂CH₂CHF₂, and —CH₂CH₂CF₃.

In some embodiments, R₉ is —S(O) R_{f}, examples of which include, but are not limited to, —S(O) CH₂F, —S(O) CHF₂, —S(O) CF₃, —S(O) CH₂CH₂F, —S(O) CH₂CHF₂, —S(O) CH₂CF₃, —S(O) CH₂CH₂CH₂F, —S(O) CH₂CH₂CHF₂, —S(O) CH₂CH₂CF₃, —S(O) CH₂CH₂CH₂CH₂F, —S(O) CH₂CH₂CH₂CHF₂, and —S(O) CH₂CH₂CH₂CF₃, with particular mention being made to —S(O) CH₂F, —S(O) CHF₂, and —S(O) CF₃.

In some embodiments, R₉ is —S(O)₂R_{f}, examples of which include, but are not limited to, —S(O)₂CH₂F, —S(O)₂CHF₂, —S(O)₂CF₃, —S(O)₂CH₂CH₂F, —S(O)₂CH₂CHF₂, —S(O)₂CH₂CF₃, —S(O)₂CH₂CH₂CH₂F, —S(O)₂CH₂CH₂CHF₂, —S(O)₂CH₂CH₂CF₃, —S(O)₂CH₂CH₂CH₂CH₂F, —S(O)₂CH₂CH₂CH₂CHF₂, and —S(O)₂CH₂CH₂CH₂CF₃, with particular mention being made to —S(O)₂CH₂F, —S(O)₂CHF₂, and —S(O)₂CF₃.

In some embodiments, R₈ and R₉ together with the nitrogen atom attached thereto are joined to form a heterocycloalkyl substituted with at least one fluorine. In some embodiments, the heterocycloalkyl group may be a 3-membered ring. In some embodiments, the heterocycloalkyl group may be a 4-membered ring. In some embodiments, the heterocycloalkyl group may be a 5-membered ring. In some embodiments, the heterocycloalkyl group may be a 6-membered ring. In some embodiments, the heterocycloalkyl group may be a 7-membered ring. In some embodiments, the heterocycloalkyl group may be an 8-membered ring. The heterocycloalkyl group contains a minimum of one nitrogen ring atom (the nitrogen atom intervening R₈ and R₉), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, with particular mention being made to aziridine, azetidine, pyrrolidine, and piperidine. When R₈ and R₉ together with the nitrogen atom attached thereto are joined to form a heterocycloalkyl group, the heterocycloalkyl group may be substituted with one fluorine atom, two fluorine atoms, three fluorine atoms, four fluorine atoms, or more. In some embodiments, the heterocycloalkyl group is substituted with two fluorine atoms.

Examples of heterocycloalkyl groups substituted with at least one fluorine atom formed from joining R₈ and R₉ together with the nitrogen atom attached thereto include, but are not limited to,

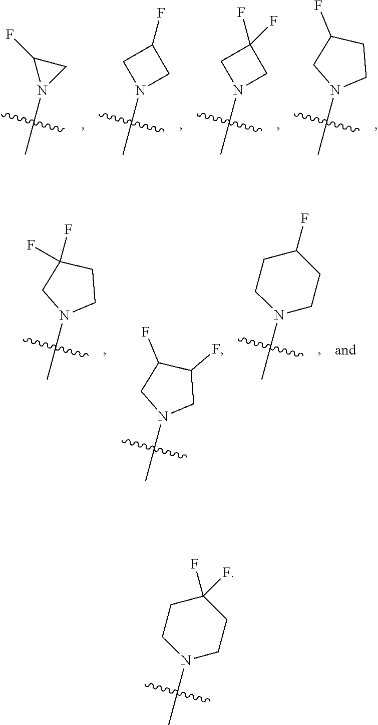

In some embodiments, R₈ and R₉ are not joined with the nitrogen atom attached thereto to form a heterocycloalkyl substituted with at least one fluorine, e.g., those exemplified above. In some embodiments, R₈ and R₉ are not joined with the nitrogen atom attached thereto to form a heterocycloalkyl substituted with at least one fluorine, e.g., those exemplified above, when X₁, X₂, Y₁, and Y₂ are hydrogen. In some embodiments, R₈ and R₉ are not joined with the nitrogen atom attached thereto to form a 4,4-difluoropiperidinyl group (represented below) when X₁, X₂, Y₁, and Y₂ are hydrogen.

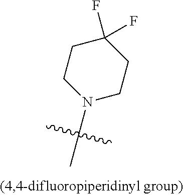 [SMILES: *N1CCC(F)(F)CC1]

R_{f} represents a fluoroalkyl group. In the compound of Formula (V), at least one of R₈ and R₉ comprises the fluoroalkyl group, R_{f}. Each R_{f} present in the disclosed compounds is independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium. In some embodiments, each H^{x} is hydrogen. In some embodiments, each H^{x} is deuterium. In some embodiments, at least one H^{x} is deuterium and at least one H^{x} is hydrogen. In some embodiments, n is 0. In some embodiments, n is 1. In some embodiments, n is 2. In some embodiments, n is 3.

Examples of R_{f} include, but are not limited to, —CH₂F, —CHF₂, —CF₃, —CH₂CH₂F, —CH₂CHF₂, —CH₂CF₃, —CD₂CH₂F, —CD₂CHF₂, —CD₂CF₃, —CH₂CH₂CH₂F, —CH₂CH₂CHF₂, —CH₂CH₂CF₃, —CD₂CH₂CH₂F, —CD₂CH₂CHF₂, —CD₂CH₂CF₃, —CD₂CD₂CH₂F, —CD₂CD₂CHF₂, —CD₂CD₂CF₃, —CH₂CH₂CH₂CH₂F, —CH₂CH₂CH₂CHF₂, —CH₂CH₂CH₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, the compound comprises one R_{f} group. In some embodiments, R₉ comprises the fluoroalkyl group, R_{f}, and R₈ represents a group that does not comprise the fluoroalkyl group, R_{f}, for example where R₈ is —CH₃ or CD₃. In some embodiments, both R₈ and Ry comprise the fluoroalkyl group, R_{f}, which may be the same or different.

In some embodiments, the compound, e.g., the compound of Formula (V), is selected from the group consisting of:

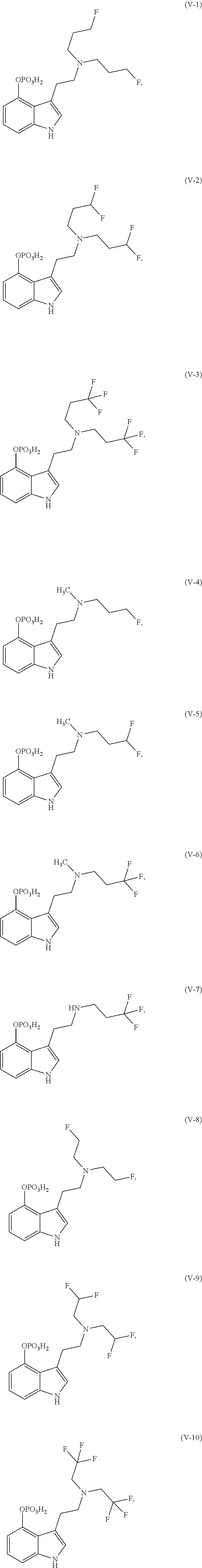

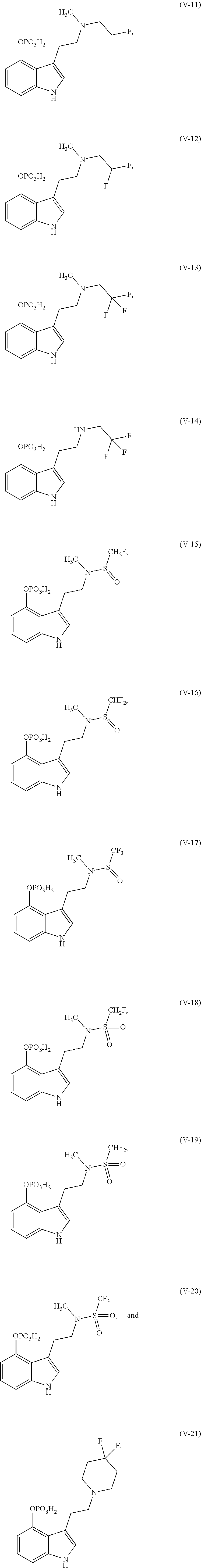

or a pharmaceutically acceptable salt, stereoisomer, tautomer, solvate, polymorph, or prodrug thereof.

The compound of Formula (I) through (V) may contain a stereogenic center. In such cases, the compounds may exist as different stereoisomeric forms, even though Formula (I) through (V) are drawn without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by, e.g., stereospecific synthesis, as is known in the art.

In some embodiments, the compound described herein, e.g., a compound of Formula (I) through (V), is non-stereogenic. In some embodiments, the compound described herein, e.g., a compound of Formula (I) through (V), is racemic. In some embodiments, the compound described herein, e.g., a compound of Formula (I) through (V), is enantiomerically enriched (one enantiomer is present in a higher percentage), including enantiomerically pure. In some embodiments, the compound described herein, e.g., a compound of Formula (I) through (V), is provided as a single diastereomer. In some embodiments, the compound described herein, e.g., a compound of Formula (I) through (V), is provided as a mixture of diastereomers. When provided as a mixture of diastereomers, the mixtures may include equal mixtures, or mixtures which are enriched with a particular diastereomer (one diastereomer is present in a higher percentage than another).

In some embodiments, the compound of Formula (I) through (V) is an agonist of a serotonin 5-HT₂ receptor.

In some embodiments, the compound of Formula (I) through (V) is an agonist of a serotonin 5-HT_{2A} receptor.

Also disclosed herein is a pharmaceutically acceptable salt of the compounds of the present disclosure, e.g., a compound of Formula (I) through (V). When the pharmaceutically acceptable salt is an acid addition salt, the acid used to form the pharmaceutically acceptable salt of the compound of Formula (I) through (V) may be a monoacid, a diacid, a triacid, a tetraacid, or may contain a higher number of acid groups. The acid groups may be, e.g., a carboxylic acid, a sulfonic acid, a phosphonic acid, or other acidic moieties containing at least one replaceable hydrogen atom. Examples of acids for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

In some embodiments, the pharmaceutically acceptable salt of the compound of Formula (I) through (V) is a benzenesulfonate salt, a tartrate salt, a hemi-fumarate salt, an acetate salt, a citrate salt, a malonate salt, a fumarate salt, a succinate salt, an oxalate salt, a benzoate salt, a salicylate salt, an ascorbate salt, a hydrochloride salt, a maleate salt, a malate salt, a methanesulfonate salt, a toluenesulfonate salt, a glucuronate salt, or a glutarate salt of the compound of Formula (I) through (V). In some embodiments, the pharmaceutically acceptable salt of the compound of Formula (I) through (V) is a salt formed from a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the pharmaceutically acceptable salt of the compound of Formula (I) through (V) is a salt formed from a benzoic acid (e.g., benzoic acid, 4-acetamidobenzoic acid, 2-acetoxybenzoic acid, salicylic acid, 4-amino-salicylic acid, etc.).

In some embodiments, the pharmaceutically acceptable salt of the compound of Formula (I) through (V) is a fatty acid salt. The fatty acid used to make the fatty acid salt of the compound of Formula (I) through (V) may be a fatty monoacid or a fatty diacid, and may contain a fatty hydrocarbon portion made up of hydrogen and anywhere from 4, from 6, from 8, from 10, from 12, from 14, from 16, and up to 26, up to 24, up to 22, up to 20, up to 18 carbon atoms, which may be fully saturated or partially unsaturated. In some embodiments, the pharmaceutically acceptable salt of the compound of Formula (I) through (V) is an adipate salt, a laurate salt, a linoleate salt, a myristate salt, a caprate salt, a stearate salt, an oleate salt, a caprylate salt, a palmitate salt, a sebacate salt, an undecylenate salt, or a caproate salt of the compound of Formula (I) through (V).

Methods for preparing pharmaceutically acceptable salt forms of pharmaceutical compounds are known by those of ordinary skill in the art. In some embodiments, the method includes:
(a) suspending the compound of Formula (I) through (V) in a solvent or mixture of solvents;
(b) contacting an acid with the compound of Formula (I) through (V) to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, octane, and combinations thereof. In some embodiments, the solvent is ethanol. In some embodiments, the solvent is 1,4-dioxane. In some embodiments, the solvent is acetonitrile. In some embodiments, the solvent is tetrahydrofuran.

Suitable acids for use in the preparation of pharmaceutically acceptable acid addition salts may include those described heretofore. The acid may be an inorganic acid such as hydrochloric acid, or an organic acid, with organic acids being preferred. In some embodiments, the acid is an organic acid selected from the group consisting of ascorbic acid, citric acid, fumaric acid, maleic acid, malonic acid, (−)-L-malic acid, (+)-L-tartaric acid, methanesulfonic acid, benzenesulfonic acid, toluenesulfonic acid, benzoic acid, salicylic acid, succinic acid, oxalic acid, D-glucuronic acid, glutaric acid salt, and acetic acid. In some embodiments, the acid is an organic acid selected from the group consisting of benzenesulfonic acid, (+)-L-tartaric acid, fumaric acid, acetic acid, citric acid, malonic acid, succinic acid, oxalic acid, benzoic acid, and salicylic acid. In some embodiments, the acid is a fatty acid, such as adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc., with particular mention being made to adipic (hexandioic) acid, lauric (dodecanoic) acid, linoleic acid, myristic (tetradecanoic) acid, capric (decanoic) acid, stearic (octadecanoic) acid, oleic acid, and caprylic (octanoic) acid.

In some embodiments, a stoichiometric (or superstoichiometric) quantity of the acid is contacted with the compound of Formula (I) through (V). In some embodiments, a sub-stoichiometric (e.g., 0.5 molar equivalents) quantity of the acid is contacted with the compound of Formula (I) through (V). The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

In some embodiments, compounds of the present disclosure, e.g., a compound of Formula (I) through (V), or a pharmaceutically acceptable salt, stereoisomer, tautomer, polymorph, or prodrug thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. Solvates of the compounds herein may be in the form of isolable solvates. In one non-limiting example, as a hydrate, the compound may be a monohydrate, a dihydrate, etc. Solvates of the compounds herein also include solution-phase forms. Thus, in some embodiments, the present disclosure provides solution-phase compositions of the compounds of the present disclosure, or any pharmaceutically acceptable salts thereof, which are in solvated form, preferably fully solvated form.

In some embodiments, the compound of the present disclosure, e.g., a compound of Formula (I) through (V), or a pharmaceutically acceptable salt, stereoisomer, tautomer, solvate, or prodrug thereof, is provided in crystalline form, e.g., as determined by XRPD. Accordingly, pharmaceutical compositions may be prepared from compounds of Formula (I) through (V), in crystalline form including in one or more polymorphic forms, and may be used for treatment as set forth herein. Crystalline forms are advantageous in terms of stability and providing well-defined physical properties, which is desirable for pharmaceutical preparation and administration.

In some embodiments, the compounds of the present disclosure, or any pharmaceutically acceptable salt, stereoisomer, or solvate thereof, are provided in amorphous form, e.g., as determined by XRPD. Accordingly, pharmaceutical compositions may be prepared from compounds of Formula (I) through (V), or a pharmaceutically acceptable salt, stereoisomer, tautomer, solvate, or prodrug thereof, in one or more amorphic forms, and may be used for treatment as set forth herein. Amorphous forms typically possess higher aqueous solubility and rates of dissolution compared to their crystalline counterparts, and thus may be well suited for quick acting dosage forms adapted to rapidly release the active agent, such as for orodispersible dosage forms (ODxs), immediate release (IR) dosage forms, and the like.

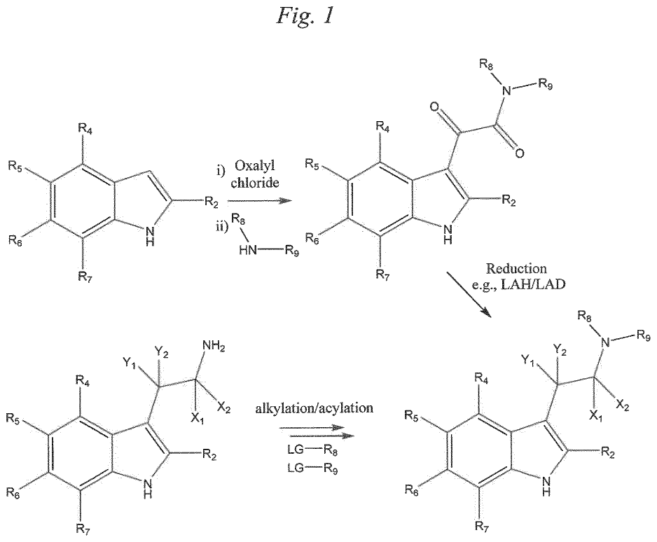

Compounds of the present disclosure, e.g., a compound of Formula (I) through (V), or a pharmaceutically acceptable salt, stereoisomer, tautomer, solvate, polymorph, or prodrug thereof, may generally be prepared according to, or analogous to, the general synthetic routes shown in FIG. 1, or the synthetic routes exemplified herein. Other synthetic routes may also be used according to techniques and procedures known to those of ordinary skill in the art.

### Pharmaceutical Compositions

Also disclosed herein is a pharmaceutical composition comprising a compound of Formula (I) through (V), or a pharmaceutically acceptable salt, stereoisomer, tautomer, solvate, polymorph, or prodrug thereof, and a pharmaceutically acceptable vehicle. The pharmaceutical compositions may contain one, or more than one, compound of the present disclosure.

“Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound or salt thereof of the present disclosure is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be water, saline, gum acacia, gelatin, starch paste, talc, keratin, colloidal silica, urea, and the like. In addition, auxiliary, stabilizing, solubilizing, thickening, lubricating, coloring agents, sweetening agents, and other pharmaceutical additives may be included in the disclosed compositions, for example those set forth hereinafter. The pharmaceutical vehicle can include an acid, such as those described heretofore for use in forming the pharmaceutically acceptable salt forms of the present disclosure, with specific mention being made to citric acid and/or tartaric acid.

The pharmaceutical composition may comprise a single compound of Formula (I) through (V) or a mixture of compounds of Formula (I) through (V). The pharmaceutical composition may be formed from an isotopologue mixture of the disclosed compounds. In some embodiments, a subject compound of Formula (I) through (V) may be present in the pharmaceutical composition at a purity of at least 50% by weight, at least 60% by weight, at least 70% by weight, at least 80% by weight, at least 90% by weight, at least 95% by weight, at least 99% by weight, based on a total weight of isotopologues of the compound of Formula (I) through (V) present in the pharmaceutical composition (i.e., the isotopic purity). In some embodiments, the composition comprises a subject compound of Formula (I) through (V), and is substantially free of other isotopologues of the subject compound, in either free base or salt form, e.g., the composition has less than 20, 15, 10, 9, 8, 7, 6, 5, 4, 3, 2 or 1 or 0.5 mole percent of other isotopologues of the subject compound.

In some embodiments, any position in the compound having deuterium has a minimum deuterium incorporation that is greater than that found naturally occurring in hydrogen (about 0.016 atom %). In some embodiments, any position in the compound having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

The pharmaceutical composition may be formulated with an enantiomerically pure compound of the present disclosure, e.g., a compound of Formula (I) through (V), or a racemic mixture of the compound(s). As described herein, a racemic compound of Formula (I) through (V) may contain about 50% of the R- and S-stereoisomers based on a molar ratio (about 48 to about 52 mol %, or about a 1:1 ratio)) of one of the isomers. In some embodiments, a composition, medicament, or method of treatment may involve combining separately produced compounds of the R- and S-stereoisomers in an approximately equal molar ratio (e.g., about 48 to 52%). In some embodiments, a medicament or pharmaceutical composition may contain a mixture of separate compounds of the R- and S-stereoisomers in different ratios. In some embodiments, the pharmaceutical composition contains an excess (greater than 50%) of the R-enantiomer. Suitable molar ratios of R/S may be from about 1.5:1, 2:1, 3:1, 4:1, 5:1, 10:1, or higher. In some embodiments, a pharmaceutical composition may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In some embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included.

The pharmaceutical composition may be formulated with one or more polymorphs of the compounds of Formula (I) through (V), or a pharmaceutically acceptable salt, stereoisomer, tautomer, solvate, or prodrug thereof, including crystalline and/or amorphous polymorphs of the compounds or salts thereof.

Pharmaceutical compositions may be generally provided herein which comprise about 0.001 to about 1000 mg, about 1 to about 500 mg, about 2 to about 100 mg, about 0.001 mg, about 0.01 mg, about 0.1 mg, about 1 mg, about 2 mg, about 3 mg, about 5 mg, about 10 mg, about 20 mg, about 30 mg, about 40 mg, about 50 mg, about 75 mg, about 100 mg, about 150 mg, about 200 mg, about 300 mg, about 400 mg, about 500 mg of one or more compounds as disclosed herein (on active basis). The quantity of compound of Formula (I) through (V)(on active basis) in a unit dose preparation may be varied or adjusted within the above ranges as deemed appropriate using sound medical judgment, according to the particular application, administration route, potency of the active ingredient, etc. The composition can, if desired, also contain other compatible therapeutic agents/active ingredients.

In some embodiments, the pharmaceutical composition comprises at least 0.1% by weight, at least 0.5% by weight, at least 1% by weight, at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, and up to 99.9% by weight, up to 99.5% by weight, up to 99% by weight, up to 98% by weight, up to 97% by weight, up to 95% by weight, up to 90% by weight, up to 85% by weight, up to 80% by weight, up to 75% by weight, up to 70% by weight, up to 65% by weight, up to 60% by weight, up to 55% by weight of the compound of Formula (I) through (V), based on a total weight of the pharmaceutical composition.

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral, intravenous, intradermal, or inhalation administration, or other routes of administration as set forth herein, to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of vehicle will be determined in part by the particular compound, and by the particular method used to administer the composition. Accordingly, there is a wide variety of suitable formulations of the subject pharmaceutical compositions. Liquid form preparations include solutions and emulsions, for example, water, water/propylene glycol solutions, or organic solvents. When administered to a mammal, the compounds and compositions of the present disclosure and pharmaceutically acceptable vehicles may be sterile. In some instances, an aqueous medium is employed as a vehicle e.g., when the subject compound is administered intravenously or via inhalation, such as water, saline solutions, and aqueous dextrose and glycerol solutions.

As described below, the pharmaceutical compositions of the present disclosure may be specially formulated for administration in solid, semi-solid, or liquid form, including those adapted for the following:
A. Oral administration, for example, drenches (aqueous or non-aqueous solutions or suspensions), tablets, films, or capsules, e.g., those targeted for buccal, sublingual, and systemic absorption, boluses, powders, granules, syrups, pastes for application to the tongue;
B. Parenteral administration, for example, by subcutaneous, intramuscular, intravenous, intradermal, or epidural injection as, for example, a sterile solution or suspension, or sustained release formulation;
C. Topical application/transdermal administration, for example, as a cream, ointment, or a controlled release patch or spray applied to the skin, or orifices and/or mucosal surfaces such as intravaginally or intrarectally, for example, as a pessary, cream or foam;
D. Modified release dosage forms, including delayed-, extended-, prolonged-, sustained-, pulsatile-, controlled-, accelerated-, fast-, targeted-, programmed-release, and gastric retention dosage forms, such modified release dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126); and
E. Inhalation administration, for example as an aerosol, preferably a mist.

Tamper resistant dosage forms/packaging of any of the disclosed pharmaceutical compositions are contemplated.

### A. Oral Administration

The pharmaceutical compositions disclosed herein may be provided in solid, semisolid, or liquid dosage forms for oral administration. As used herein, oral administration includes gastric (enteral) delivery, for example whereby the medication is taken by mouth and swallowed, as well as intraoral administration such as through the mucosal linings of the oral cavity, e.g., buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, films, sprinkles, elixirs, and syrups. In addition to the active ingredient(s), the pharmaceutical compositions may contain one or more pharmaceutically acceptable vehicles (e.g., carriers or excipients), including, but not limited to, binders, fillers, diluents, disintegrants, wetting agents, lubricants, glidants, coloring agents, dye-migration inhibitors, sweetening agents, preservatives, antioxidants, lyoprotectants, stabilizing agents, solubilizing agents, complexing agents, and flavoring agents.

In some embodiments, pharmaceutical compositions of the present disclosure may be in orodispersible dosage forms (ODxs), including orally disintegrating tablets (ODTs)(also sometimes referred to as fast disintegrating tablets, orodispersible tablets, or fast dispersible tablets) or orodispersible films (ODFs)(or wafers). Such dosage forms allow for pre-gastric absorption of the compounds herein, e.g., when administered intraorally through the mucosal linings of the oral cavity, e.g., buccal, lingual, and sublingual administration, for increased bioavailability and faster onset compared to oral administration through the gastrointestinal tract.

Orodispersible dosage forms can be prepared by different techniques, such as freeze drying (lyophilization), molding, spray drying, mass extrusion or compressing. Preferably, the orally disintegrating tablets are prepared by lyophilization. In some embodiments, the orodispersible dosage forms disintegrate in less than about 90 seconds, in less than about 60 seconds, in less than about 30 seconds, in less than about 20, in less than about 10 seconds, in less than about 5 seconds, or in less than about 2 seconds after being received in the oral cavity.

In some embodiments, the orodispersible dosage forms dissolve in less than about 90 seconds, in less than about 60 seconds, or in less than about 30 seconds after being received in the oral cavity. In some embodiments, the orodispersible dosage forms disperse in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the pharmaceutical compositions are in the form of orodispersible dosage forms, such as oral disintegrating tablets (ODTs), having a disintegration time according to the United States Phamacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Phamacopeia (USP) disintegration test <701>, such as when adapted for extended release, for example 2 minutes, 3 minutes, 4 minutes, 5 minutes, 10 minutes, 15 minutes, 20 minutes, 25 minutes, 30 minutes, 45 minutes, 60 minutes, or any range therebetween, or longer, are also contemplated.

In some embodiments, the pharmaceutical compositions are in the form of lyophilized orodispersible dosage forms, such as lyopholized ODTs. In some embodiments, the lyophilized orodispersible dosage forms (e.g., lyophilized ODTs) are created by creating a porous matrix by subliming the water from pre-frozen aqueous formulation of the drug containing matrix-forming agents and other vehicles such as those set forth herein, e.g., one or more lyoprotectants, preservatives, antioxidants, stabilizing agents, solubilizing agents, flavoring agents, etc. In some embodiments, the orodispersible dosage form comprises two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the dosage form (binder). In some embodiments, the second constituent is a matrix-supporting/disintegration-enhancing agent such as sucrose, lactose, mannitol, xylitol, microcrystalline cellulose, calcium diphosphate, and/or starch, which acts by cementing the porous framework, provided by the water-soluble polymer and accelerates the disintegration of the orodispersible dosage form. In some embodiments, the lyophilized orodispersible dosage form (e.g., lyophilized ODT) includes gelatin and mannitol. In some embodiments, the lyophilized orodispersible dosage form (e.g., lyophilized ODT) includes gelatin, mannitol, and one or more of a lyoprotectant, a preservative, an antioxidant, a stabilizing agent, a solubilizing agent, a flavoring agent, etc., with particular mention being made to citric acid. A non-limiting example of an ODT formulation is Zydis® orally dispersible tablets (available from Catalent). In some embodiments, the ODT formulation (e.g., Zydis® orally dispersible tablets) includes one or more water-soluble polymers, such as gelatin, one or more matrix materials, fillers, or diluents such as mannitol, a compound of Formula (I) through (V), or a pharmaceutically acceptable salt, stereoisomer, tautomer, solvate, polymorph, or prodrug thereof, and optionally a lyoprotectant, a preservative, an antioxidant, a stabilizing agent, a solubilizing agent, and/or a flavoring agent. In some embodiments, the ODT formulation (e.g., Zydis® orally dispersible tablets) includes gelatin, mannitol, a compound of Formula (I) through (V), or a pharmaceutically acceptable salt, stereoisomer, tautomer, solvate, polymorph, or prodrug thereof, and an organic acid, non-limiting examples of which are citric acid and/or tartaric acid, or any suitable organic acid set forth herein.

In some embodiments, the pharmaceutical composition is in the form of lyophilized orodispersible film (ODF)(or wafer). In some embodiments, the pharmaceutical compositions are in the form of lyophilized ODFs protected for the long-term storage by a specialty packaging excluding moisture, oxygen, and light. In some embodiments, the lyophilized ODFs are created by creating a porous matrix by subliming the water from pre-frozen aqueous formulation of the drug containing matrix-forming agents and other vehicles such as those set forth herein, e.g., one or more of a lyoprotectant, a preservative, an antioxidant, a stabilizing agent, a solubilizing agent, a flavoring agent, etc. In some embodiments, the lyophilized ODF includes a thin water-soluble film matrix. In some embodiments, the ODFs comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the film/wafer (binder). In some embodiments, the second constituent is matrix-supporting/disintegration-enhancing agents such as sucrose and mannitol, which acts by cementing the porous framework, provided by the water-soluble polymer and accelerates the disintegration of the wafer. In some embodiments, the lyophilized ODFs include gelatin and mannitol. In some embodiments, the lyophilized ODFs include gelatin, mannitol, and one or more of a lyoprotectant, a preservative, an antioxidant, a stabilizing agent, a solubilizing agent, a flavoring agent, etc., with particular mention being made to citric acid.

In some embodiments, the ODF (or wafer) can comprise a monolayer, bilayer, or trilayer. In some embodiments, the monolayer ODF contains an active agent and one or more pharmaceutically acceptable vehicles (e.g., carrier or excipients). In some embodiments, the bilayer ODF contains one or more excipients, such as a solubilizing agent, in a first layer and an active agent in the second layer. This configuration allows the active agent to be stored separately from the excipients and can increase the stability of the active agent and optionally increase the shelf life of the composition compared to the case where the excipients and the active agent were contained in a single layer. For tri-layer ODFs, each of the layers may be different or two of the layers, such as the upper and lower layers, may have substantially the same composition. In some embodiments, the lower and upper layers surround a core layer containing the active agent. In some embodiments, the lower and upper layers may contain one or more excipients, such as a solubilizing agent. In some embodiments, the lower and upper layers have the same composition. Alternatively, the lower and upper layers may contain different excipients or different amounts of the same excipient. The core layer typically contains the active agent, optionally with one or more excipients.

In some embodiments, in addition to the active ingredient(s), the pharmaceutical compositions in orodispersible dosage forms (ODxs) may contain one or more pharmaceutically acceptable vehicles (e.g., carriers or excipients). For example, in some embodiments, pharmaceutical compositions in orodispersible dosage forms include one or more of pharmaceutically acceptable a lyoprotectant, a preservative, an antioxidant, a stabilizing agent, a solubilizing agent, a flavoring agent, etc.

Examples of pharmaceutically acceptable lyoprotectants include, but are not limited to, disaccharides such as sucrose and trehalose, anionic polymers such as sulfobutylether-β-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include: (1) water soluble antioxidants, such as ascorbic acid, cysteine or salts thereof (cysteine hydrochloride), sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, glycerol, methionine, monothioglycerol, ascorbic acid, citric acid, polysorbate, arginine, cyclodextrins, microcrystalline cellulose, modified celluloses (e.g., carboxymethylcellulose, sodium salt), sorbitol, and cellulose gel.

Examples of pharmaceutically acceptable solubilizing agents (or dissolution aids) include, but are not limited to, citric acid, hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, fumaric acid, DL-malic acid, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, tartaric acid, dioctylsodium sulfosuccinate, zein, powdered skim milk, sorbitan trioleate, lactic acid, aluminum lactate, ascorbyl palmitate, hydroxyethylmethylcellulose, hydroxypropylmethylcelluloseacetate succinate, polyoxyethylene (105) polyoxypropylene (5)glycol, polyoxyethylene hydrogenated castor oil 60, polyoxyl 35 castor oil, poly(sodium 4-styrenesulfonate), polyvinylacetaldiethylamino acetate, polyvinyl alcohol, maleic acid, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate. Of these, in some embodiments, such as in ODT formulation, citric acid is preferred.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, lemon, lime, and lemon-lime.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Pharmaceutical compositions adapted for oral administration, e.g., tablets including compressed tablets, may be formulated with various vehicles such as those set forth herein. Examples of suitable vehicles may include, but are not limited to, binders, fillers, diluents, disintegrants, wetting agents, lubricants, glidants, coloring agents, dye-migration inhibitors, sweetening agents, preservatives, antioxidants, stabilizing agents, solubilizing agents, and flavoring agents.

Binders or granulators impart cohesiveness to a tablet to ensure the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present, e.g., from about 10%, about 20%, about 30%, about 40%, about 50%, about 60%, about 70%, about 80%, about 90%, about 99% by weight in the pharmaceutical compositions disclosed herein, or any range therebetween.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain, e.g., from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; sodium stearyl fumarate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-O-SIL® (Cabot Co. of Boston, Mass.); and mixtures thereof. The pharmaceutical compositions disclosed herein may contain, e.g., about 0.1 to about 5% by weight of a lubricant.

Suitable glidants include colloidal silicon dioxide, CAB-O-SIL® (Cabot Co. of Boston, Mass.), and asbestos-free talc.

Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Sweetening agents include sucrose, lactose, mannitol, syrups, glycerin, and artificial sweeteners, such as saccharin and aspartame.

Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

Suspending and dispersing agents include sodium carboxymethylcellulose, pectin, tragacanth, Veegum, acacia, sodium carbomethylcellulose, hydroxypropyl methylcellulose, and polyvinylpyrolidone.

Preservatives include glycerin, methyl and propylparaben, benzoic add, sodium benzoate and alcohol.

Wetting agents include propylene glycol monostearate, sorbitan monooleate, diethylene glycol monolaurate, and polyoxyethylene lauryl ether.

Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many vehicles (carriers, excipients, etc.) may serve several functions, even within the same formulation. Particular mention is made to pharmaceutical compositions herein containing citric acid, which may play multiple roles as a stabilizing agent, as a solubilizing agent to provide fast dissolution of the active for rapid onset, etc., particularly for dosage forms adapted for rapid onset and a shorter duration of drug action, such as orodispersible dosage forms (e.g., ODTs and ODFs).

The pharmaceutical compositions herein may be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more vehicles (e.g., carriers or excipients) described herein, including binders, disintegrants, controlled-release polymers, lubricants, diluents, and/or colorants. Flavoring and sweetening agents are especially useful in the formation of chewable tablets and lozenges.

Disclosed herein are pharmaceutical compositions in modified release dosage forms, which comprise a compound as disclosed herein and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Further disclosed herein are pharmaceutical compositions in enteric coated dosage forms, which comprise a compound as disclosed herein and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise a compound as disclosed herein and one or more release controlling excipients or carriers for use in an effervescent dosage form. The pharmaceutical compositions may also comprise non-release controlling excipients or carriers.

Additionally disclosed are pharmaceutical compositions in a dosage form that has an instant releasing component and at least one delayed releasing component, and is capable of giving a discontinuous release of the compound in the form of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise a compound as disclosed herein and one or more release controlling and non-release controlling excipients or carriers, such as those excipients or carriers suitable for a disruptable semipermeable membrane and as swellable substances.

Disclosed herein also are pharmaceutical compositions in a dosage form for oral administration to a subject, which comprise a compound, salt, or solvate as disclosed herein and one or more pharmaceutically acceptable vehicles (e.g., excipients or carriers), enclosed in an intermediate reactive layer comprising a gastric juice-resistant polymeric layered material partially neutralized with alkali and having cation exchange capacity and a gastric juice-resistant outer layer.

The dosage form may be an immediate release (IR) dosage form, examples of which include, but are not limited to, an immediate release (IR) tablets or an immediate release (IR) capsule. In addition to the active ingredient (e.g., a compound of Formula (I) through (V)), dosage forms adapted for immediate release may include one or more pharmaceutically acceptable vehicles which readily disperse, dissolve, or otherwise breakdown in the gastric environment so as not to delay or prolong dissolution/absorption of the active ingredient(s).

Examples of pharmaceutically acceptable vehicles for immediate release dosage forms include, but are not limited to, one or more binders/granulators, matrix materials, fillers, diluents, disintegrants, dispersing agents, solubilizing agents, lubricants, and/or performance modifiers. In some embodiments, the immediate release (IR) dosage form is an immediate release (IR) tablet comprising one or more of microcrystalline cellulose, sodium carboxymethylcellulose, magnesium stearate, mannitol, crospovidone, and sodium stearyl fumarate. In some embodiments, the immediate release (IR) dosage form comprises microcrystalline cellulose, sodium carboxymethylcellulose, and magnesium stearate. In some embodiments, the immediate release (IR) dosage form comprises mannitol, crospovidone, and sodium stearyl fumarate.

The pharmaceutical compositions disclosed herein may be disclosed as soft or hard capsules, which can be made from gelatin, methylcellulose, starch, or calcium alginate. The hard gelatin capsule, also known as dry-filled capsule (DFC) or powder in capsule (PIC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The pharmaceutical compositions disclosed herein may be disclosed in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein (e.g., a compound of Formula (I) through

(V)), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

The pharmaceutical compositions disclosed herein may be disclosed as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in any of the disclosed dosage forms.

The pharmaceutical compositions disclosed herein may be co-formulated with other active ingredients which do not impair the desired therapeutic action, or with substances that supplement the desired action, such as hydrocortisone.

### B. Parenteral Administration

The pharmaceutical compositions disclosed herein may be administered parenterally by injection, infusion, perfusion, or implantation, for local or systemic administration. Parenteral administration, as used herein, includes, but is not limited to, intravenous, intradermal, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable vehicles (e.g., carriers and excipients), including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, methyl-β-cyclodextrin, hydroxypropyl-3-cyclodextrin/hydroxypropyl-β-cyclodextrin, sulfobutylether-β-cyclodextrin, and sulfobutylether 7-O-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In some embodiments, the pharmaceutical compositions are disclosed as ready-to —use sterile solutions. In some embodiments, the pharmaceutical compositions are disclosed as sterile dry soluble products, including lyophilized powders and hypodermic tablets, to be reconstituted with a vehicle prior to use. In some embodiments, the pharmaceutical compositions are disclosed as ready-to —use sterile suspensions. In some embodiments, the pharmaceutical compositions are disclosed as sterile dry insoluble products to be reconstituted with a vehicle prior to use. In some embodiments, the pharmaceutical compositions are disclosed as ready-to —use sterile emulsions.

The pharmaceutical compositions may be formulated as a suspension, solid, semi-solid, or thixotropic liquid, for administration as an implanted depot. In some embodiments, the pharmaceutical compositions disclosed herein are dispersed in a solid inner matrix, which is surrounded by an outer polymeric membrane that is insoluble in body fluids but allows the active ingredient in the pharmaceutical compositions to diffuse through. Fatty acid salts of the compounds of Formula (I) through (V) may be well-suited for such dosage forms.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions disclosed herein may be administered topically to the skin, orifices, or mucosa. The effects may be local or systemic. Topical administration, as described herein, includes, but is not limited to, conjuctival, intracorneal, intraocular, ophthalmic, auricular, transdermal, nasal (e.g., intranasal), vaginal, uretheral, respiratory, and rectal administration.

The pharmaceutical compositions disclosed herein may be formulated in any dosage form that is suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may contain the active ingredient(s) which may be mixed under sterile conditions with a pharmaceutically acceptable vehicle, and with any preservatives, buffers, absorption enhancers, propellants which may be required. Liposomes, micelles, microspheres, nanosystems, and mixtures thereof, may also be used.

Pharmaceutically acceptable vehicles (e.g., carriers and excipients) suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The ointments, pastes, creams and gels may contain, in addition to an active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient(s), excipients such as lactose, talc, silicic acid, aluminum hydroxide, calcium silicates and polyamide powder, or mixtures of these substances. Sprays, such as those used for (intra) nasal administration, can additionally contain customary propellants, such as chlorofluorohydrocarbons and volatile unsubstituted hydrocarbons, such as butane and propane.

Transdermal delivery devices (e.g., patches) may be used. Such dosage forms have the added advantage of providing controlled delivery of active ingredient(s) to the body. That is, the compounds of the present disclosure (e.g., a compound of Formula (I) through (V), or a pharmaceutically acceptable salt, stereoisomer, tautomer, solvate, polymorph, or prodrug thereof) can be administered via a transdermal patch at a steady state concentration, whereby the active ingredient(s) is gradually administered over time, thus avoiding drug spiking and adverse events/toxicity associated therewith.

Transdermal patch dosage forms herein may be formulated with various amounts of the active ingredient(s), depending on the disease/condition being treated, the active ingredient(s) employed, the permeation and size of the transdermal delivery device, the release time period, etc. For example, when formulated with a compound of Formula (I) through (V), a unit dose preparation may be varied or adjusted e.g., from 5 mg, 10 mg, 15 mg, 20 mg, 25 mg, 30 mg, 35 mg, 40 mg, 45 mg, 50 mg, to 200 mg, 175 mg, 150 mg, 125 mg, 100 mg, 95 mg, 90 mg, 85 mg, 80 mg, 75 mg, 70 mg, 65 mg, 60 mg, 55 mg of the compound of Formula (I) through (V) (active basis) or otherwise as deemed appropriate using sound medical judgment, according to the particular application and the potency of compound.

Transdermal patches formulated with the disclosed compounds may be suitable for microdosing or sub-psychedelic (also referred to herein as sub-psychoactive) dosing, to achieve durable therapeutic benefits, with decreased toxicity. In some embodiments, the compound of Formula (I) through (V), or a pharmaceutically acceptable salt, stereoisomer, tautomer, solvate, polymorph, or prodrug thereof, is administered via a transdermal patch at sub-psychoactive (yet still potentially serotonergic) concentrations, for example, over an extended period such as over a 8, 24, 48, 72, 84, 96, or 168 hour time period.

In addition to the active ingredient(s), and any optional pharmaceutically acceptable vehicles(s), the transdermal patch may also include one or more of a pressure sensitive adhesive layer, a backing, and a release liner, as is known to those of ordinary skill in the art.

Transdermal patch dosage forms can be made by dissolving or dispersing the compounds herein in the proper medium. In some embodiments, the compounds of the present disclosure may be dissolved/dispersed directly into a polymer matrix forming the pressure sensitive adhesive layer. Such transdermal patches are called drug-in-adhesive (DIA) patches. Preferred DIA patch forms are those in which the active ingredient(s) is distributed uniformly throughout the pressure sensitive adhesive polymer matrix. In some embodiments, the active ingredient(s) may be provided in a layer containing the active ingredient(s) plus a polymer matrix which is separate from the pressure sensitive adhesive layer. In any case, the compounds of the present disclosure may optionally be formulated with suitable vehicles(s) such as carrier agents, permeation agents/absorption enhancers, humectants/crystallization inhibitors, etc. to increase the flux across the skin.

Examples of carrier agents may include, but are not limited to, C₈-C₂₂ fatty acids, such as oleic acid, undecanoic acid, valeric acid, heptanoic acid, pelargonic acid, capric acid, lauric acid, and eicosapentaenoic acid; C₈-C₂₂ fatty alcohols such as octanol, nonanol, oleyl alcohol, decyl alcohol and lauryl alcohol; lower alkyl esters of C₈-C₂₂ fatty acids such as ethyl oleate, isopropyl myristate, butyl stearate, and methyl laurate; di(lower) alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy) ethanol; diethylene glycol monomethyl ether; alkylaryl ethers of polyethylene oxide; polyethylene oxide monomethyl ethers; polyethylene oxide dimethyl ethers; glycerol; ethyl acetate; acetoacetic ester; N-alkylpyrrolidone; cyclodextrins, such as α-cyclodextrin, β-cyclodextrin, γ-cyclodextrin, or derivatives such as 2-hydroxypropyl-β-cyclodextrin; and terpenes/terpenoids, such as limonene, linalool, myrcene, pinene such as α-pinene, caryophyllene, citral, eucolyptol, and the like; including mixtures thereof.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Examples of humectants/crystallization inhibitors include, but are not limited to, polyvinyl pyrrolidone-co-vinyl acetate, HPMC, polymethacrylate, and mixtures thereof.

The pressure sensitive adhesive layer may be formed from polymers including, but not limited to, acrylics (polyacrylates including alkyl acrylics), polyvinyl acetates, natural and synthetic rubbers (e.g., polyisobutylene), ethylenevinylacetate copolymers, polysiloxanes, polyurethanes, plasticized polyether block amide copolymers, plasticized styrene-butadiene rubber block copolymers, and mixtures thereof. The pressure-sensitive adhesive layer used in the transdermal patch of the present disclosure may be formed from an acrylic polymer pressure-sensitive adhesive, preferably an acrylic copolymer pressure sensitive adhesive. The acrylic copolymer pressure sensitive adhesive may be obtained by copolymerization of one or more alkyl (meth)acrylates (e.g., 2-ethylhexyl acrylate); aryl(meth)acrylates; arylalkyl (meth)acrylate; and (meth)acrylates with functional groups such as hydroxyalkyl (meth)acrylates (e.g., hydroxyethyl acrylate, 2-hydroxypropyl acrylate, 3-hydroxypropyl acrylate, 4-hydroxybutyl acrylate, 2-hydroxyethyl methacrylate, 2-hydroxypropyl methacrylate, 3-hydroxypropyl methacrylate, and 4-hydroxybutyl methacrylate), carboxylic acid containing (meth)acrylates (e.g., acrylic acid), and alkoxy(meth)acrylates (e.g., methoxyethyl acrylate); optionally with one or more copolymerizable monomers (e.g., vinylpyrrolidone, vinyl acetate, etc.). Specific examples of acrylic pressure-sensitive adhesives may include, but are not limited to, DURO-TAK products (Henkel) such as DURO-TAK 87-900A, DURO-TAK 87-9301, DURO-TAK 87-4098, DURO-TAK 87-2074, DURO-TAK 87-235A, DURO-TAK 87-2510, DURO-TAK 87-2287, DURO-TAK 87-4287, DURO-TAK 87-2516, DURO-TAK 387-2052, and DURO-TAK 87-2677.

The backing used in the transdermal patch of the present disclosure may include flexible backings such as films, nonwoven fabrics, Japanese papers, cotton fabrics, knitted fabrics, woven fabrics, and laminated composite bodies of a nonwoven fabric and a film. Such a backing is preferably composed of a soft material that can be in close contact with a skin and can follow skin movement and of a material that can suppress skin rash and other discomforts following prolonged use of the patch. Examples of the backing materials include, but are not limited to, polyethylene, polypropylene, polyethylene terephthalate, polybutylene terephthalate, polyethylene naphthalate, polystyrene, nylon, cotton, acetate rayon, rayon, a rayon/polyethylene terephthalate composite body, polyacrylonitrile, polyvinyl alcohol, acrylic polyurethane, ester polyurethane, ether polyurethane, a styrene-isoprene-styrene copolymer, a styrene-butadiene-styrene copolymer, a styrene-ethylene-propylene-styrene copolymer, styrene-butadiene rubber, an ethylene-vinyl acetate copolymer, or cellophane, for example. Preferred backings do not adsorb or release the active ingredient(s). In order to suppress the adsorption and release of the active ingredient(s), to improve transdermal absorbability of the active ingredient(s), and to suppress skin rash and other discomforts, the backing preferably includes one or more layers composed of the material above and has a water vapor permeability. Specific examples of backings may include, but are not limited to, 3M COTRAN products such as 3M COTRAN ethylene vinyl acetate membrane film 9702, 3M COTRAN ethylene vinyl acetate membrane film 9716, 3M COTRAN polyethylene membrane film 9720, 3M COTRAN ethylene vinyl acetate membrane film 9728, and the like.

The release liner used in the transdermal patch of the present disclosure may include, but is not limited to, a polyester film having one side or both sides treated with a release coating, a polyethylene laminated high-quality paper treated with a release coating, and a glassine paper treated with a release coating. The release coating may be a fluoropolymer, a silicone, a fluorosilicone, or any other release coating known to those of ordinary skill in the art. The release liner may have an uneven surface in order to easily take out the transdermal patch from a package. Examples of release liners may include, but are not limited to SCOTCHPAK products from 3M such as 3M SCOTCHPAK 9744, 3M SCOTCHPAK 9755, 3M SCOTCHPAK 9709, and 3M SCOTCHPAK 1022.

Other layers such as abuse deterrent layers formulated with one or more irritants (e.g., sodium lauryl sulfate, poloxamer, sorbitan monoesters, glyceryl monooleates, spices, etc.), may also be employed.

Methods disclosed herein using a transdermal patch dosage form provide for systemic delivery of small doses of active ingredient(s), preferably over extended periods of time such as up to 168 hour time periods, for example from 2 to 96 hours, or 4 to 72 hours, or 8 to 24 hours, or 10 to 18 hours, or 12 to 14 hours. In particular, the compound of Formula (I) through (V) can be delivered in small, steady, and consistent doses such that deleterious or undesirable side-effects can be avoided. In some embodiments, the compound of Formula (I) through (V) is administered transdermally at sub-psychoactive (yet still potentially serotonergic concentrations) concentrations.

An exemplary drug-in-adhesive (DIA) patch formulation may comprise 5 to 30 wt. % of a compound of Formula (I) through (V), or a pharmaceutically acceptable salt, stereoisomer, tautomer, solvate, polymorph, or prodrug thereof, 30 to 70 wt. % pressure sensitive adhesive (e.g., DURO-TAK 387-2052, DURO-TAK 87-2677, and DURO-TAK 87-4098), 1 to 10 wt. %

permeation agents/absorption enhancers (e.g., oleyloleate, oleyl alcohol, levulinic acid, diethylene glycol monoethyl ether, etc.), and 5 to 35 wt. % crystallization inhibitor (e.g., polyvinyl pyrrolidone-co-vinyl acetate, HPMC, polymethacrylate, etc.), each based on a total weight of the DIA patch formulation, though it should be understood that many variations are possible in light of the teachings herein.

Automatic injection devices offer a method for delivery of the compositions disclosed herein to patients. The compositions disclosed herein may be administered to a patient using automatic injection devices through a number of known devices, a non-limiting list of which includes transdermal, subcutaneous, and intramuscular delivery.

In some transdermal, subcutaneous, or intramuscular applications, a composition disclosed herein is absorbed through the skin. Passive transdermal patch devices often include an absorbent layer or membrane that is placed on the outer layer of the skin. The membrane typically contains a dose of a substance that is allowed to be absorbed through the skin to deliver the composition to the patient. Typically, only substances that are readily absorbed through the outer layer of the skin may be delivered with such transdermal patch devices.

Other automatic injection devices disclosed herein are configured to provide for increased skin permeability to improve delivery of the disclosed compositions. Non-limiting examples of structures used to increase permeability to improve transfer of a composition into the skin, across the skin, or intramuscularly include the use of one or more microneedles, which in some embodiments may be coated with a composition disclosed herein. Alternatively, hollow microneedles may be used to provide a fluid channel for delivery of the disclosed compositions below the outer layer of the skin. Other devices disclosed herein include transdermal delivery by iontophoresis, sonophoresis, reverse iontophoresis, or combinations thereof, and other technologies known in the art to increase skin permeability to facilitate drug delivery.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally. The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, including, but not limited to, fluorohydrocarbons, chlorofluorohydrocarbons, and volatile unsubstituted hydrocarbons, such as butane, propane, 1,1,1,2-tetrafluoroethane, and/or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

The pharmaceutical compositions disclosed herein may be micronized to a size suitable for delivery, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as l-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavor, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-, controlled-, targeted, and programmed release.

### D. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

In some embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT),′ CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the composition.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical compositions disclosed herein are formulated as AMT controlled-release dosage forms, which comprises an asymmetric osmotic membrane that coats a core comprising the active ingredient(s) and other pharmaceutically acceptable vehicles (e.g., excipients or The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 m to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems.

### E. Inhalation Administration

The pharmaceutical compositions disclosed herein may be formulated for inhalation administration, e.g., for pulmonary absorption. Suitable preparations may include liquid form preparations such as those described above, e.g., solutions and emulsions, wherein the solvent or carrier is, for example, water, water/water-miscible vehicles such as water/propylene glycol solutions, or organic solvents, with optional buffering agents, which can be delivered as an aerosol, preferably a mist, with a carrier gas, such as air, oxygen, a mixture of helium and oxygen, or other gases and gas mixtures. The pharmaceutical compositions may also be formulated as a dry powder for insufflation, alone or in combination with an inert carrier such as lactose or phospholipids.

The pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable as propellant, such dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases.

Aqueous solutions suitable for inhalation use can be prepared by dissolving the compound of Formula (I) through (V), or a pharmaceutically acceptable salt, stereoisomer, tautomer, solvate, polymorph, or prodrug thereof, in water or other water-based medium. Suitable stabilizers and thickening agents can also be added. Emulsions suitable for inhalation use can be made by solubilizing the compound of Formula (I) through (V), or a pharmaceutically acceptable salt, stereoisomer, tautomer, solvate, polymorph, or prodrug thereof, in an aqueous medium and dispersing the solubilized form in a hydrophobic medium, optionally with viscous material, such as natural or synthetic gums, resins, methylcellulose, sodium carboxymethylcellulose, and other suspending agents.

Solutions or suspensions for use in a pressurized container, pump, spray, atomizer, or nebulizer may be formulated to contain a surfactant or other appropriate co-solvent, or a suitable alternative agent for dispersing, solubilizing, or extending release of the active ingredient disclosed herein, and optionally a propellant. Such surfactants or co-solvents may include, but are not limited to, Polysorbate 20, 60, and 80; Pluronic F-68, F-84, and P-103; cyclodextrin; polyoxyl 35 castor oil; sorbitan trioleate, oleic acid, or an oligolactic acid. Surfactants and co-solvents may be optionally employed at a level between about 0.01% and about 2% by weight of the pharmaceutical composition. Viscosity greater than that of simple aqueous solutions may be desirable in some cases to decrease variability in dispensing the formulations, to decrease physical separation of components of an emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents, when desirable, are typically employed at a level between about 0.01% and about 2% by weight of the pharmaceutical composition.

The compounds of the present disclosure can also be dissolved in organic solvents or aqueous mixtures of organic solvents. Organic solvents can be, for example, acetonitrile, chlorobenzene, chloroform, cyclohexane, 1,2-dichloromethane, dichloromethane, 1,2-dimethoxyethane, N,N-dimethylacetamide, N,N-dimethylformamide, 1,4-dioxane, 2-ethoxyethanol, ethylene glycol, formamide, hexane, methanol, ethanol, 2-methoxyethanol, methybutylketone, methylcyclohexane, N-methylpyrrolidone, nitromethane, pyridine, sulfolane, tetralin, toluene, 1,1,2-trichloroethylene, or xylene, and like, including combinations thereof. Organic solvents can belong to functional group categories such as ester solvents, ketone solvents, alcohol solvents, amide solvents, ether solvents, hydrocarbon solvents, etc. each of which can be used.

The compounds of the present disclosure (e.g., a compound of Formula (I) through (V)) can be delivered as an aerosol, preferably a mist, via inhalation, for systemic administration to the patient's central nervous system. Preferably, the aerosol is generated without externally added heat (this does not exclude minor temperature increases caused by the formation of the aerosol itself, such as with a vibrating mesh or other nebulizer. However, such minor temperature increases can often be offset by vaporization of the drug, which results in cooling of the composition). The compounds of the present disclosure can be delivered as an aerosol, preferably a mist, with a carrier, such as air, oxygen, or a mixture of helium and oxygen, or other gas mixtures including therapeutic gas mixtures. The carrier gas, e.g., air, oxygen, a mixture of helium and oxygen, or other gases and gas mixtures, can be heated to about 50° C. to about 60° C., or to about 55° C. to about 56° C. When a mixture of helium and oxygen is used as the carrier, the helium can be present in the mixture of oxygen and helium at about 50%, 60%, 70%, 80% or 90% by volume, and the oxygen can be present in the mixture at about 50%, 40%, 30%, or 10% by volume, or any range therebetween.

Inhalation delivery can further comprise administering a pretreatment inhalation therapy prior to administration of the aerosol comprising the compound of Formula (I) through (V). The pretreatment can comprise administering via inhalation of a mixture of helium and oxygen heated to about 90° C., to about 92° C., to about 94° C., to about 96° C., to about 98° C., to about 100° C., to about 105° C., to about 110° C., to about 115° C., to about 120° C., or any range therebetween, to the patient. For example, an inhalation procedure may involve (i) administering via inhalation a mixture of helium and oxygen heated to about 90° C. to about 120° C. to the patient, followed by (ii) administering via inhalation a mixture of helium and oxygen heated to about 50° C. to about 60° C. and the aerosol comprising the compound of Formula (I) through (V) to the patient and then repeating steps (i) and (ii). Steps (i) and (ii) can be repeated 1, 2, 3, 4, 5, or more times.

The compounds of the present disclosure (e.g., compound of Formula (I) through (V)) can, in some embodiments, be administered via aerosol inhalation at doses of about 1 μg to about 200 mg or more (or any range between about 1 μg to about 200 mg), e.g., about 1 μg, 2 μg, 5 μg, 6 μg, 10 μg, 13 μg, 15 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 g, 190 μg, 200 μg, 210 μg, 220 μg, 230 μg, 240 μg, 250 μg, 260 μg, 270 μg, 280 μg, 290 μg, 300 μg, 400 μg, 500 μg, 1.0 mg, 2.0 mg, 3.0 mg, 4.0 mg, 5.0 mg, 6.0 mg, 7.0 mg, 8.0 mg, 9.0 mg, 10.0 mg, 20.0 mg, 30.0 mg, 40.0 mg, 50.0 mg, 60.0 mg, 70.0 mg, 80.0 mg, 90.0 mg, 100.0 mg, 150.0 mg, 200.0 mg, or more, per inhalation session. In some embodiments, a subject can have 1, 2, 3, 4, 5 or more inhalation sessions a day. In some embodiments, a subject can have 1, 2, 3, 4, 5 or more inhalation sessions every other day, once a week, twice a week, or three times a week. In some embodiments, a subject can have 1, 2, 3, 4, 5 or more inhalation sessions every other month, twice a month, three times a month, or four times a month. In some embodiments, a subject can have 1, 2, 3, 4, 5, 6, 7, 8, or more inhalation sessions per treatment course, such as within a 28-day time period.

### Aerosols

An aerosol, preferably a mist, can be delivered using, air, oxygen, a mixture of helium and oxygen, or other gases and gas mixtures, as a carrier gas. The carrier gas can be delivered at room temperature or heated. In some embodiments, an aerosol, preferably a mist comprising a compound of Formula (I) through (V) is delivered via inhalation using heated helium-oxygen (HELIOX) mixtures. Due to very low viscosity of helium the helium-oxygen mixtures generate gaseous streams characterized by laminar flow that is a highly desirable feature for reaching out into the deep lung areas and reducing deposition of the drug in the respiratory tract, one of the major obstacles in dose delivery via inhalation. A patient can inhale a dissolved compound disclosed herein as a mist into an alveolar region of the patient's lungs. The compound of

Formula (I) through (V) can then be delivered to a fluid lining of the alveolar region of the lungs and can be systemically absorbed into patient blood circulation. Advantageously, these formulations can be effectively delivered to the blood stream upon inhalation to the alveolar regions of the lungs.

Devices suitable for delivery of heated or unheated carrier gas (e.g., air, oxygen, or helium-oxygen mixtures) include, for example, continuous mode nebulizers Flo-Mist (Phillips) and Hope (B&B Medical Technologies) and the accessories such as regulators, e.g., Medipure™ Heliox-LCQ System (PraxAir) and control box, e.g., Precision Control Flow (PraxAir). In some embodiments, a full delivery setup can be a device as described in, for example, Russian patent RU199823U1.

The term “heliox” as used herein refers to breathing gas mixtures of helium gas (He) and oxygen gas (O₂). In some embodiments, the heliox mixture can contain helium in the mixture of helium and oxygen at about 50%, 60%, 70%, 80% or 90% by volume, and contain oxygen in the mixture of helium and oxygen at about 50%, 40%, 30%, or 10% by volume, or any range therebetween. The heliox mixture can thus contain helium and oxygen in a volume ratio of 50:50, 60:40, 70:30, 80:20, 90:10, or any range therebetween. In some embodiments, heliox can generate less airway resistance through increased tendency to laminar flow and reduced resistance in turbulent flow.

The use of heat in heliox mixtures can further enhance drug delivery by increasing permeability of key physical barriers for drug absorption. Heating of mucosal surfaces can increase permeability by enhancing peripheral blood circulation and relaxing the interstitial junction, as well as other mechanisms. Helium has a thermal conductivity almost 10 times higher than oxygen and nitrogen and can facilitate heat transfer more efficiently. A dry heliox mixture can be used safely as a pretreatment step when warmed up to as high as 110° C., which can enable the dry heliox mixture to heat mucosal surfaces of the lung and respiratory tract more efficiently.

Various types of personal vaporizers are known in the art. In general, personal vaporizers are characterized by heating a solid drug or compound. Vaporizers can work by directly heating a solid drug or compound to a smoldering point. Vaporizing a solid or solid concentrate can be done by convection or conduction. Convection heating of solid concentrate involves a heating element coming into contact with water, or another liquid, which then vaporizes. The hot vapor in turn directly heats the solid or solid concentrate to a smoldering point, releasing a vapor to be inhaled by a user. Conduction heating involves direct contact between the solid or solid concentrate and the heating element, which brings the solid to a smoldering point, releasing vapor to be inhaled by a user. Though vaporizers present advantages over smoking in terms of lung damage, the drug/active ingredient that is vaporized can be substantially deteriorated by the vaporizing heat.

In some embodiments, the compound of Formula (I) through (V) is delivered via a nebulizer, which generates an aqueous-droplet aerosol, preferably a mist, containing the compound, which is optionally combined with a heated helium-oxygen mixture. In some embodiments, the disclosed compounds are delivered via a nebulizer, which generates an aqueous-droplet aerosol, preferably a mist, containing the compound, which is combined with a driving gas comprising nitrous oxide. The driving gas comprising nitrous oxide may be nitrous oxide gas itself or a therapeutic gas mixture, such as a N₂O—O₂ mixture or a N₂O-air mixture. The therapeutic gas mixture may further include other gases such as one or more of N₂, Ar, CO₂, Ne, CH₄, He, Kr, H₂, Xe, H₂O (e.g., vapor), etc. In some embodiments, the driving gas is a therapeutic gas mixture comprising N₂O, which is present at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture, or any range in between. The presence of nitrous oxide (being an NMDA receptor antagonist) in (or as) the driving gas can augment the effect of the disclosed compounds and provide the ability to use lower doses thereof to obtain similar levels of effect.

For example, a preparation of compound of Formula (I) through (V), or a pharmaceutically acceptable salt, stereoisomer, tautomer, solvate, polymorph, or prodrug thereof, can be placed into a liquid medium and put into an aerosol by a device, such as a nebulizer. In some embodiments, a nebulizer can be, for example, a pneumatic compressor nebulizer, an ultrasonic nebulizer, a vibrating mesh or horn nebulizer, or a microprocessor-controlled breath-actuated nebulizer. In some embodiments, a nebulizer device can be a device as described in, for example, Russian patent RU199823U1.

A nebulizer is a device that turns a drug, such as a compound of Formula (I) through (V), in solution or suspension into a fine aerosol, such as a mist, for delivery to the lungs. A nebulizer can also be referred to as an atomizer. To atomize is to put a dissolved drug into an aerosol, such as a mist, form. To deliver a drug by nebulization, a drug can be dispersed in a liquid medium, for example, water, ethanol, or propylene glycol. Additionally, the disclosed compounds can be carried in a vehicle such as, for example liposomes, polymers, emulsions, micelles, nanoparticles, or polyethylenimine (PEI). Liquid drug formations for nebulizers can be, for example, aqueous solutions or viscous solutions. After application of a dispersing forcer (e.g., jet of gas, ultrasonic waves, or vibration of mesh), the dissolved drug is contained within liquid droplets, which are then inhaled. A mist can contain liquid droplets containing the drug in air or another gaseous mixture (e.g., a mixture of helium and oxygen).

Jet nebulizers (also known as pneumatic nebulizers or compressor nebulizers) use compressed gas to make a mist. In some embodiments, a jet nebulizer is a microprocessor-controlled breath-actuated nebulizer, also called a breath-actuated nebulizer. A breath-actuated nebulizer creates a mist only when a patient is inhaling, rather than creating a mist continuously. A mist can be generated by, for example, passing air flow through a Venturi in a nebulizer bowl or cup. A Venturi is a system for speeding the flow of a fluid by constricting fluid in a cone shape tube. In the restriction, the fluid must increase its velocity, thereby reducing its pressure and producing a partial vacuum. As the fluid exits the constriction point, its pressure increases back to the ambient or pipe level pressure. This can form a low-pressure zone that pulls up droplets through a feed tube from a solution of drug in a nebulizer bowl, and in turn this creates a stream of atomized droplets, which flow to a mouthpiece. Higher air flows lead to a decrease in particle size and an increase in output. Due to droplets and solvent that saturates the outgoing gas, jet nebulizers can cool a drug solution in the nebulizer and increase solute concentration in the residual volume. A baffle in a nebulizer bowl or cup can be impacted by larger particles, retaining them and returning them to the solution in the nebulizer bowl or cup to be reatomized. Entrainment of air through a nebulizer bowl as the subject inhales can increase mist output during inspiration. Generation of a mist can occur with a smaller particle size distribution, but using smaller particle sizes can result in an increased nebulization time.

The unit of measurement generally used for droplet size is mass median diameter (MMD), which is defined as the average droplet diameter by mass. This unit can also be referred to as the mass mean aerodynamic diameter, or MMAD. The MMD droplet size for jet nebulizers can be about 1.0, 1.5, 2.0, 2.5, 3.0, 3.5, 4.0, 5.0, 6.0, 7.0, 8.0, 9.0, 10.0 μm or more (or any range between about 1.0 and 10.0 μm), which can be smaller than that of ultrasonic nebulizers.

Ultrasonic nebulizers generate mists by using the vibration of a piezoelectric crystal, which converts alternating current to high-frequency (about 1 to about 3 MHz) acoustic energy. The solution breaks up into droplets at the surface, and the resulting mist is drawn out of the device by the patient's inhalation or pushed out by gas flow through the device generated by a small compressor. Ultrasonic nebulizers can include large-volume ultrasonic nebulizers and small-volume ultrasonic nebulizers. Droplet sizes tend to be larger with ultrasonic nebulizers than with jet nebulizers. The MMD droplet size for ultrasonic nebulizers can be about 2.0, 2.5, 3.0, 3.5, 4.0, 4.5, 5.0, 5.5, 6.0, 6.5, 7.0, 7.5, 8.0, 9.0, 10.0 μm or more (or any range between about 2.0 and 10.0 μm). Ultrasonic nebulizers can create a dense mist, with droplets at about 100, 150, 200, 250, 300 μm/L or more.

Mesh nebulizer devices use the vibration of a piezoelectric crystal to indirectly generate a mist. Mesh nebulizers include, for example, active mesh nebulizers and passive mesh nebulizers. Active mesh nebulizers use a piezo element that contracts and expands on application of an electric current and vibrates a precisely drilled mesh in contact with the drug solution to generate a mist. The vibration of a piezoelectric crystal can be used to vibrate a thin metal plate perforated by several thousand holes. One side of the plate is in contact with the liquid to be atomized, and the vibration forces this liquid through the holes, generating a mist of tiny droplets. Passive mesh nebulizers use a transducer horn that induces passive vibrations in the perforated plate with tapered holes to produce a mist. Examples of active mesh nebulizers include the Aeroneb® (Aerogen, Galway, Ireland) and the eFlow® (PARI, Starnberg, Germany), while the Microair NE-U22® (Omron, Bannockburn, IL) is a passive mesh nebulizer. Mesh nebulizers are precise and customizable. By altering the pore size of the mesh, the device can be tailored for use with drug solutions of different viscosities, and the output rate changed. Use of this method of atomization can offer several advantages. The size of the droplets can be extremely precise because droplet size can be determined by the size of the holes in the mesh (which may be tailor-made to suit the application). Nebulizer meshes can be manufactured using methods such as electrodeposition, electroplating, and laser cutting to produce a liquid particle in gas in the respirable range. Mesh can be made of metal alloy. The metals used in mesh manufacture can include platinum, palladium, nickel, and stainless steel. The size of the droplet is about twice the size of the mesh hole. Mesh holes, therefore, can be about 0.1, 0.5, 1.0, 1.5, 2.0, 2.5, 3.0, 3.5, 4.0, 4.5, 5.0 μm or more (or any value in between about 0.1 and 5.0 μm). Mist generation in mesh nebulizers can vary based on the shape of the mesh, the material that the mesh is made of, and also the way that the mesh is created. In other words, different meshes can produce different sized liquid particles suspended in gas. Generally, MMD droplet size for mesh nebulizers can be about 1.0, 1.5, 2.0, 2.5, 3.0, 3.5, 4.0, 4.5, 5.0, 5.5., 6.0, 6.1, 6.2, 6.3, 6.4, 6.5, 6.6, 6.7, 6.8, 6.9, 7.0 μm or more (or any value in between about 1.0 and 7.0 μm).

Additionally, droplet size can be programmable. In particular, geometric changes can be made to a nebulizer to provide a specific desired droplet size. Additionally, droplet size can be controlled independently of droplet velocity. The volume of liquid atomized, and the droplet velocity can also be precisely controlled by adjusting the frequency and amplitude of the mesh vibration. Furthermore, the number of holes in the mesh and their layout on the mesh can be tailored. Mesh nebulizers can be powered either by electricity or by battery.

A mist output rate in standing cloud mL per minute (for any atomization methodology described herein) can range from, for example, 0.1, 0.2. 0.3, 0.4, 0.5, 0.6, 0.7, 0.8, 0.9 mL/minute or more (or any range between about 0.1 and 0.9 mL/minute) and the residual volume in any type of nebulizer reservoir can range from a about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mL or more (or any range between about 0.01 and 2.0 mL). Precise droplet size control can be advantageous since droplet size can correlate directly to kinetic drug release (KDR). Precise control of KDR can be achievable with precise control of droplet size. The compounds herein can be delivered via a mist using any methodology with an MMD droplet size of about 0.5, 1.0, 1.5, 2.0, 2.5, 3.0, 3.5, 4.0, 5.0, 6.0, 7.0, 8.0, 9.0, 10.0 μm or more (or any range between about 0.5 and 10.0 μm).

In some embodiments, a compound of Formula (I) through (V) can be delivered via a continuous positive airway pressure (CPAP) or other pressure-assisted breathing device. A pressure-assisted breathing device forces a continuous column of compressed air or other gas at a fixed designated pressure against the face and nose of the patient, who is wearing a mask or nasal cap. When the patient's glottis opens to inhale, the pressure is transmitted throughout the airway, helping to open it. When the patient exhales, pressure from the deflating lungs and chest wall pushes air out against the continuous pressure, until the two pressures are equal. The air pressure in the airway at the end of exhalation is equal to the external air pressure of the machine, and this helps “splint” the airway open, allowing better oxygenation and airway recruitment. A pressure-assisted breathing device can be coupled with a means for introducing mist particles into the gas flow in the respiratory circuit and or a means for discontinuing the introduction of mist particles into the respiratory circuit when the patient exhales. See, e.g. U.S. Pat. No. 7,267,121.

In some embodiments, a mist can be delivered by a device such as a metered dose inhaler (MDI)(also referred to as a pressurized metered dose inhaler or pMDI), which generates an organic solvent-droplet mist containing a compound of Formula (I) through (V), which is optionally combined with a heated helium-oxygen mixture. In some embodiments, the compound can be delivered via a metered dose inhaler, MDI. MDI devices can include a canister which contains the compound of Formula (I) through (V) and a propellant, a metering valve which dispenses the medicament from the canister, an actuator body that receives the canister and which forms an opening for oral inhalation, and an actuator stem which receives the drug from the canister and directs it out the opening in the actuator body. A non-limiting example of a metering valve and actuator is Bespak's BK357 valve and actuator (orfice d=0.22 mm) by Recipharm. Moving the drug canister relative to the actuator body and actuator stem causes the metering valve to release the predetermined amount of the drug. In some embodiments, the compound of Formula (I) through (V), or a pharmaceutically acceptable salt, stereoisomer, tautomer, solvate, polymorph, or prodrug thereof, can be dissolved in a liquid propellant mixture (sometimes including small amounts of a volatile organic solvent) stored in a pressurized container of the MDI. The “metered dose” is the dose that is prepackaged in a single-dose inhaler, or which in a multidose inhaler is automatically measured out of a reservoir in preparation for inhalation. MDI devices can be aided with spacers. An MDI spacer is a spacer that goes between the MDI and the mouth of a user of the MDI. An MDI spacer allows droplets in the atomized dose to settle out a bit and mix with air or other gas, thus allowing for more effective delivery of a metered dose into a user's lungs when inhaled. An MDI spacer assists in preventing a user from inhaling the metered dose directly from an MDI where the dose would be traveling so fast that the droplets of the atomized spray from the MDI hit and stick to the back of the user's throat rather than being inhaled into the user's lungs where the drug of the metered dose is designed to be delivered. MDI devices offer the advantage of regular dosing, which can be controlled in the manufacture of the drug.

Drugs can also be delivered by dry powder inhalers (DPI). In such DPI devices, the drug itself can form the powder or the powder can be formed from a pharmaceutically acceptable excipient or carrier and the drug is releasably bound to a surface of the carrier powder such that upon inhalation, the moisture in the lungs releases the drug from the surface to make the drug available for systemic absorption. In some embodiments, the compound of Formula (I) through (V), or a pharmaceutically acceptable salt, stereoisomer, tautomer, solvate, polymorph, or prodrug thereof, is delivered by use of a dry powder inhaler (DPI). Depending on the compound or form used, the drug can be formed into the necessary powder itself, or can be releasably bound to a surface of a carrier powder. Such carrier powders are known in the art (see, e.g., H. Hamishehkar, et al., “The Role of Carrier in Dry Powder Inhaler”, Recent Advances in Novel Drug Carrier Systems, 2012, pp. 39-66).

DPI is generally formulated as a powder mixture of coarse carrier particles and micronized drug particles with aerodynamic particle diameters of 1-5 μm (see e.g., Iida, Kotaro, et al. “Preparation of dry powder inhalation by surface treatment of lactose carrier particles” Chemical and pharmaceutical bulletin 51.1 (2003): 1-5). Carrier particles are often used to improve drug particle flowability, thus improving dosing accuracy and minimizing the dose variability observed with drug formulations alone while making them easier to handle during manufacturing operations. Carrier particles should have several characteristics such as physico-chemical stability, biocompatibility and biodegradability, compatible with the drug substance and must be inert, available and economical. The choice of carrier particle (both content and size) is well within the purview of one of ordinary skill in the art. The most common carrier particles are made of lactose or other sugars, with α-lactose monohydrate being the most common lactose grade used in the inhalation field for such particulate carriers.

Delivery with Helium Oxygen Mixtures

Systemic delivery of the compound of Formula (I) through (V) can be carried out via inhalation of an aerosol comprising the compound and a carrier gas such as air, oxygen, helium, a mixture of helium and oxygen (i.e., a heliox mixture), other gases or other gas mixtures. In some embodiments, the carrier gas can be heated. The method can further comprise using a device containing a balloon with an oxygen-helium mixture equipped with a reducer and a mask connected to each other by a gas or air connecting tube, which contains an additional heating element capable of heating the gas mixture up to 120° C., a nebulizer with a vibrating porous plate or mesh, ensuring the passage of droplets with a size of less than 5 microns through it, and a disinfection unit.

In some embodiments, the compound of Formula (I) through (V) is delivered to the lower respiratory tract, for instance, to a pulmonary compartment such as alveoli, alveolar ducts and/or bronchioles. From there, the drug can enter the blood stream and travel to the central nervous system. In some embodiments, inhalation of a mist can deliver the compound of Formula (I) through (V) to the patient's CNS without passing through the liver. Administration via inhalation can allow gaseous drugs or those dispersed in a liquid or a mist, to rapidly reach the blood stream, bypassing first-pass metabolism. First-pass metabolism, also known as “first-pass effect” or “presystemic metabolism” describes drugs that enter the liver and undergo extensive biotransformation.

In some embodiments, the present disclosure provides a treatment step, in which the compound of Formula (I) through (V) can be administered to a patient in need thereof by administering via inhalation a mixture of helium and oxygen heated to about 50° C., 51° C., 52° C., 53° C., 54° C., 55° C., 56° C., 57° C., 58° C., 59° C., 60° C., or more (or any range between 50° C. to 60° C.) and the atomized compound. In some embodiments, a mist or vapor of the compound can have a particle size from about 0.1 microns to about 10 microns (e.g., about 10, 5, 4, 3, 2, 1, 0.1 or less microns). In some embodiments, atomization is performed via a nebulizer creating an inhalant that is a mist. In some embodiments, the atomized compound is driven down the patient delivery line by the patient's inhalation. In some embodiments, the atomized compound is driven down the patient delivery line by the patient's inhalation using a carrier gas. The carrier gas can be air, oxygen, a mix of oxygen and helium, heated air, heated oxygen, a heated helium and oxygen mixture, among others.

In some embodiments, the treatment step can be preceded by a pretreatment step. In some embodiments, the pretreatment step can comprise first administering a pretreatment inhalation therapy prior to administration of the mist of the compound of Formula (I) through

(V). In some embodiments, the pretreatment inhalation step can comprise (i) administering via inhalation air, oxygen, or mixture of helium and oxygen heated to about 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., or more (or any range between about 90° C. and 120° C.) and no drug, and then (ii) administering a treatment step of inhalation air, oxygen, a mix of oxygen and helium, heated air, heated oxygen, or heated helium and oxygen mixture, with the atomized compound of Formula (I) through (V). Heated air, heated oxygen, or heated helium and oxygen mixture, in combination with the atomized compound of the present disclosure, can be heated to about 50° C., 51° C., 52° C., 53° C., 54° C., 55° C., 56° C., 57° C., 58° C., 59° C., 60° C., or more (or any range between about 50° C. and 60° C.).

In some embodiments of the present disclosure, a pretreatment step (i) and a treatment step (ii) can be repeated 0, 1, 2, 3, 4, 5, or more times. In some embodiments of the present disclosure, steps (i) and (ii) can be repeated 0, 1, 2, 3, 4, 5, or more times followed by the treatment step, which can be repeated 0, 1, 2, 3, 4, 5, or more times. In some embodiments of the present disclosure, the treatment step can be repeated 0, 1, 2, 3, 4, 5, or more times with no pretreatment step.

Treatment, with optional pretreatment, can be administered once a week, twice a week, once a day, twice a day, three times a day or more, a defined number of treatments per treatment course (e.g., 1, 2, 3, or 4 treatments per treatment course) or other treatment schedules as set forth herein.

A drug delivery procedure can comprise an inhaled priming no-drug hot heliox mixture to effectively preheat the mucosal bed followed by inhaling an atomized compound of the present disclosure, again driven by the heated heliox, but at lower temperatures, that are now dictated by lower heat tolerance to the wet vs. dry inhaled gas stream. Consequently, this procedure can be conducted in multiple repeated cycles, wherein a target PK and drug exposure is controlled by the concentration of the drug, temperature, flow rate of the helium oxygen mixture, composition of the mixture, number and durations of cycles, time and combinations of the above.

Combination Therapy with an NMDA Receptor Antagonist

Also disclosed are combination drug therapies based on administration of both a compound of Formula (I) through (V), or a pharmaceutically acceptable salt, stereoisomer, tautomer, solvate, polymorph, or prodrug thereof, (as a 5-HT_{2A} receptor agonist) and a N-methyl-D-aspartate (NMDA) receptor antagonist. The combination drug therapy may enhance activity and improve patient experience when treating diseases or disorders associated with 5-HT_{2A} and/or NMDA receptors (e.g., a neuropsychiatric disease or disorder, a central nervous system (CNS) disorder, a psychological disorder, etc.), for example, by providing therapeutic efficacy while reducing or eliminating psychiatric adverse effects such as acute psychedelic crisis (bad trip) and dissociative effects from hallucinogens (out of body experience).

Non-limiting examples of NMDA receptor antagonists include, but are not limited to, ketamine, nitrous oxide, memantine, amantadine, dextromethorphan (DXM), phencyclidine (PCP), methoxetamine (MXE), dizocilpine (MK-801), esmethadone, or a combination thereof. In particular, nitrous oxide (N₂O), commonly known as laughing gas, is a rapid and effective analgesic gas that has a fast onset and rarely produces side effects when administered under proper medical supervision. Nitrous oxide is also a dissociative inhalant known to cause euphoria during inhalation. Prominent effects of nitrous oxide are increased feelings of euphoria, a heightened pain threshold, and involuntary laughing. Furthermore, unlike ketamine, nitrous oxide is not addictive. For these reasons, the use of nitrous oxide as the NMDA receptor antagonist is preferred.

In some embodiments, the combination drug therapy involves providing the compound of Formula (I) through (V), or a pharmaceutically acceptable salt, stereoisomer, tautomer, solvate, polymorph, or prodrug thereof, and the NMDA receptor antagonist as a single dosage form for administration to a patient (e.g., each is combined to provide a single aerosol that is inhaled by the patient; or each is combined into a single transdermal patch and delivered transdermally or subcutaneously to the patient). For example, when the NMDA receptor antagonist is nitrous oxide, the compound of Formula (I) through (V), or a pharmaceutically acceptable salt, stereoisomer, tautomer, solvate, polymorph, or prodrug thereof, may be present in the liquid phase of the aerosol, while the nitrous oxide may be present in the gas phase of the aerosol. The nitrous oxide (or therapeutic gas mixture comprising nitrous oxide) may be used in the generation of the aerosol or as a carrier gas used to deliver a generated aerosol to the patient. When a generated aerosol is combined with a carrier gas, the carrier gas becomes a part of the gas phase of the aerosol, i.e., the liquid phase of the aerosol becomes entrained in/diluted by the carrier gas. In some embodiments, the combination drug therapy involves providing the compound of Formula (I) through (V), or a pharmaceutically acceptable salt, stereoisomer, tautomer, solvate, polymorph, or prodrug thereof, and the NMDA receptor antagonist as separate dosage forms. For example, the compound of Formula (I) through (V), or a pharmaceutically acceptable salt, stereoisomer, tautomer, solvate, polymorph, or prodrug thereof, may be provided as an aerosol, preferably a mist, while the NMDA receptor antagonist is provided separately as a therapeutic gas mixture. Alternatively, the compound of Formula (I) through (V), or a pharmaceutically acceptable salt, stereoisomer, tautomer, solvate, polymorph, or prodrug thereof, may be provided as an injectable (e.g., intravenous, intradermal, etc.), bolus, infusion, perfusion, etc., while the NMDA receptor antagonist is provided as a therapeutic gas mixture for inhalation delivery.

The co-action of the compound of Formula (I) through (V)(as a 5-HT_{2A} receptor agonist) and a NMDA receptor antagonist (e.g., nitrous oxide, ketamine, etc.) may provide multiple benefits. For example, the NMDA receptor antagonist may control and/or reduce the activating effects of the 5-HT₂R₅, thereby reducing the risk of overstimulation and occurrences of psychiatric adverse effects such as acute psychedelic crisis. Additionally, administration of the NMDA receptor antagonist may enable the use of a reduced therapeutic dose of the compound of Formula (I) through (V), thereby decreasing the likelihood of a negative patient experience or dose-dependent side effects. Similarly, administration of the compound of Formula (I) through (V) may reduce the amount of NMDA receptor antagonist necessary for a therapeutic effect, which in the case of NMDA receptor antagonists such as nitrous oxide may alleviate certain side effects such as induced involuntary laughter and the general feelings of anxiety associated therewith. Thus, it is believed that co-administration would reduce the likelihood of a negative experience from the psychedelic administration, either because less psychedelic would be administered or the NMDA receptor antagonist (e.g., nitrous oxide, ketamine, etc.) would enable more efficient functioning of the psychedelic. Similarly, such co-administration would reduce the time or amount of NMDA receptor antagonist (e.g., nitrous oxide, ketamine, etc.) necessary for a therapeutic effect.

NMDA receptor antagonists (e.g., nitrous oxide) and 5-HT_{2A} receptor agonists function via different pharmacological pathways. However, both pathways appear to ultimately converge in a cascade at mTOR (mammalian target of rapamycin, or mechanistic target of rapamycin). Thus, a shared mechanism of action appears to exist between NMDA receptor antagonists and 5-HT_{2A} receptor agonists. Specifically, mTOR's signaling pathway may be modulated by 5-HT_{2A} receptor activation and NMDA antagonism. Without being bound by theory, such modulation of the mTOR pathway may underpin the immediate and long-lasting therapeutic and synergistic benefits of combined administration of both agents. As such, in some embodiments, administration of both agents at psychedelic or sub-psychedelic doses enables therapeutic efficacy without or minimizing psychiatric adverse effects.

In addition, it has been found that atrophy of neurons in the prefrontal cortex (PFC) plays a key role in the pathophysiology of depression and related disorders. The ability to promote both structural and functional plasticity in the PFC has been hypothesized to underlie the fast-acting antidepressant properties of the dissociative anesthetic ketamine but also the long-lasting effect after a single administration. Without being bound by theory, it is believed that the combination drug therapy disclosed herein may function by synergistically increasing neuritogenesis and spinogenesis, including increased density of dendritic spines, thereby providing or contributing to long-lasting therapeutic benefits.

A ratio of the compound of Formula (I) through (V) and the NMDA receptor antagonist administered in the combination drug therapy may vary depending on the patient (i.e., subject), the identity of the active ingredient(s) selections of the combination, the dosage form(s), and the specific disease or condition being treated. It should be understood that a specific ratio of the combination for any particular patient will depend upon a variety of factors, such as the activity of the specific compounds employed, the age, sex, general health of the patient, time of administration, rate of excretion, and the severity of the particular disease or condition being treated. In some embodiments, a weight ratio of the compound of Formula (I) through (V) and the NMDA receptor antagonist administered to the patient may range from about 1:100 to about 100:1, or any range therebetween, e.g., from about 1:75, from about 1:50, from about 1:40, from about 1:30, from about 1:20, from about 1:10, from about 1:8, from about 1:6, from about 1:5, from about 1:4, from about 1:3, from about 1:2, from about 2:3, from about 1:1, and up to about 100:1, up to about 75:1, up to about 50:1, up to about 40:1, up to about 30:1, up to about 20:1, up to about 10:1, up to about 8:1, up to about 6:1, up to about 5:1, up to about 4:1, up to about 3:1, up to about 2:1. Ratios outside of this range may also be employed, in certain circumstances.

The combination drug therapy is intended to embrace administration of the compound of Formula (I) through (V) and the NMDA receptor antagonist (e.g., nitrous oxide) in a sequential manner, that is, wherein each active ingredient is administered at a different time, as well as administration of these active ingredients, or at least two of the active ingredients, in a concurrent manner. Concurrent administration can be accomplished, for example, by administering to the subject a single dosage form having a fixed ratio of each active ingredient or in multiple, single dosage forms for each of the active ingredients. Administration of the compound of Formula (I) through (V) and a NMDA receptor antagonist (e.g., nitrous oxide), whether in a single dosage form or separate dosage forms, can be carried out by any administration route set forth herein. In some embodiments, both the compound of Formula (I) through (V) and the NMDA receptor antagonist are administered via inhalation, preferably in aerosol (e.g., mist) form. In some embodiments, the compound of Formula (I) through (V) is administered intravenously (IV), and the NMDA receptor antagonist is administered via inhalation. In some embodiments, the compound of Formula (I) through (V) is administered orally, and the NMDA receptor antagonist is administered via inhalation. In some embodiments, both the compound of Formula (I) through (V) and the NMDA receptor antagonist are administered transdermally or subcutaneously. The compositions for inhalation such as pharmaceutically acceptable vehicles, etc. for the single or separate dosage forms are set forth herein.

In some embodiments, the NMDA receptor antagonist used in the combination drug therapy is nitrous oxide. Nitrous oxide may be administered alone, or as a therapeutic gas mixture, e.g., N₂O and O₂; N₂O and air; N₂O and medical air (medical air being 78% nitrogen, 21% oxygen, 1% other gases); N₂O and a N₂/O₂ mix; N₂O and O₂ enriched medical air; N₂O and a He/O₂ mix etc. Thus, in addition to nitrous oxide and oxygen, the therapeutic gas mixture may further include other gases such as one or more of N₂, Ar, CO₂, Ne, CH₄, He, Kr, H₂, Xe, H₂O (e.g., vapor), etc. For example, nitrous oxide may be administered using a blending system that combines N₂O, O₂ and optionally other gases from separate compressed gas cylinders into a therapeutic gas mixture which is delivered to a patient via inhalation. Alternatively, the therapeutic gas mixture containing nitrous oxide may be packaged, for example, in a pressurized tank or in small, pressurized canisters which are easy to use and/or portable. The blending system and/or pressurized tanks/canisters may be adapted to fluidly connect to an inhalation device such as a device capable of generating an aerosol of the compound of Formula (I) through (V). Nitrous oxide itself, or the therapeutic gas mixture comprising nitrous oxide may be used for the generation of the aerosol (i.e., as the gas phase component of the aerosol) or as a carrier gas to facilitate the transfer of a generated aerosol to a patient's lungs. In some embodiments, N₂O is present in the therapeutic gas mixture at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture. The therapeutic gas mixture containing nitrous oxide can be administered over any desired duration, e.g., 5 minutes, 10 minutes 15 minutes, 20 minutes, 30 minutes, 40 minutes, 45 minutes, 50 minutes, 60 minutes, 90 minutes, 120 minutes, 150 minutes, 180 minutes, or any range therebetween.

In some embodiments, the compound of Formula (I) through (V) and the NMDA receptor antagonist (e.g., nitrous oxide) are each delivered by aerosol inhalation, as a single dosage form or as separate dosage forms. The aerosol, preferably a mist, may be generated by any capable device (e.g., a pressurized container, pump, spray, atomizer, or nebulizer), such as those devices disclosed herein, with or without the use of a propellant. When nitrous oxide is administered concurrently with the compound of Formula (I) through (V), the nitrous oxide may dually act as a carrier gas or propellant for the aerosol generation and as a therapeutic agent (an NMDA receptor antagonist).

In some embodiments, the delivery device is an inhalation delivery device for delivery of the combination of the compound of Formula (I) through (V) and nitrous oxide by inhalation to a patient in need thereof, comprising an inhalation outlet portal for administration of the combination to the patient; a container configured to deliver nitrous oxide, e.g., in a therapeutic gas mixture, to the inhalation outlet portal; and a device configured to generate and deliver an aerosol comprising the compound of Formula (I) through (V) to the inhalation outlet portal. In some embodiments, the inhalation outlet portal is selected from a mouthpiece or a mask covering the patient's nose and mouth. In some embodiments, the device configured to generate and deliver the aerosol to the inhalation outlet portal is a nebulizer. In some embodiments, the nebulizer is a jet nebulizer and the nitrous oxide gas, alone, or in combination with other gases (therapeutic gas mixture containing nitrous oxide), acts as a driving gas for the jet nebulizer. Accordingly, nitrous oxide delivered using a nebulizer (e.g., jet nebulizer) may dually act as a therapeutic agent and as a driving gas to entrain the nebulized form of the compound of Formula (I) through (V).

In some embodiments, the device is a dual delivery device configured to administer the compound of Formula (I) through (V), preferably in the form of an aerosol, and to simultaneously administer a controlled amount of nitrous oxide, either alone or as a therapeutic gas mixture. Any of the above aerosol delivery devices can be used for such a device, with the addition of a source of nitrous oxide (or a source of a therapeutic gas mixture containing nitrous oxide) configured to provide a metered, controlled dose/flow rate of nitrous oxide through the same administration outlet as the aerosol delivery device. In some embodiments, the driving gas for the nebulization of the compound of Formula (I) through (V) is the nitrous oxide or therapeutic gas mixture containing nitrous oxide.

Any of the delivery devices above, e.g., controlled release device, implant, patch, pump, depot, inhaler, inhalation delivery device, etc., can be optionally manufactured with smart technology, e.g., electronics, configured to provide remote activation and operational control of the drug delivery. The remote activation can be performed via computer or mobile app. To ensure security, the remote activation device can be password encoded. This technology enables a healthcare provider to perform telehealth sessions with a patient, during which the healthcare provider can remotely activate and administer the compound of Formula (I) through (V), or a pharmaceutically acceptable salt, stereoisomer, tautomer, solvate, polymorph, or prodrug thereof, via the desired delivery device while supervising the patient on the televisit.

### Therapeutic Applications and Methods

Also disclosed is a method of treating a subject with a disease or disorder comprising administering to the subject a therapeutically effective amount of a compound of Formula (I) through (V), or a pharmaceutically acceptable salt, stereoisomer, tautomer, solvate, polymorph, or prodrug thereof. In some embodiments, the disease or disorder is associated with a serotonin 5-HT₂ receptor.

The dosage and frequency (single or multiple doses) of the compounds herein administered can vary depending upon a variety of factors, including, but not limited to, the compound/salt form/polymorph to be administered; the disease/condition being treated; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards (e.g., up-titration) or downwards (e.g., down-titration).

Dosages may be varied depending upon the requirements of the subject and the active ingredient (e.g., a compound of Formula (I) through (V)) being employed. The dose administered to a subject, in the context of the pharmaceutical compositions presented herein, should be sufficient to affect a beneficial therapeutic response in the subject over time. The size of the dose also will be determined by the existence, nature, and extent of any adverse side effects. Generally, treatment is initiated with smaller dosages, which are less than the optimum dose of the active ingredient. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Routes of administration may include oral routes (e.g., enteral/gastric delivery, intraoral administration such buccal, lingual, and sublingual routes), parenteral routes (e.g., intravenous, intradermal, intraarterial, intraperitoneal, intrathecal, intraventricular, intraurethral, intrasternal, intracranial, intramuscular, intrasynovial, and subcutaneous administration), topical routes (e.g., conjuctival, intracorneal, intraocular, ophthalmic, auricular, transdermal, nasal (e.g., intranasal), vaginal, uretheral, respiratory, and rectal administration), inhalation, or others sufficient to affect a beneficial therapeutic response.

Administration may follow a continuous administration schedule, or an intermittent administration schedule. The administration schedule may be varied depending on the active ingredient(s) employed, the condition being treated, the administration route, etc. For example, administration of a compound of Formula (I) through (V), or a pharmaceutically acceptable salt, stereoisomer, tautomer, solvate, polymorph, or prodrug thereof, may be performed once a day (QD), or in divided dosages throughout the day, such as 2-times a day (BID), 3-times a day (TID), 4-times a day (QID), or more. In some embodiments administration may be performed nightly (QHS). In some embodiments, administration is performed as needed (PRN). Administration may also be performed on a weekly basis, e.g., once a week, twice a week, three times a week, four times a week, every other week, every two weeks, etc., or less. The administration schedule may also designate a defined number of treatments per treatment course, for example, the compound of Formula (I) through (V), or a pharmaceutically acceptable salt, stereoisomer, tautomer, solvate, polymorph, or prodrug thereof, may be administered 1 time, 2 times, 3 times, 4 times, 5 times, 6 times, 7 times, or 8 times per treatment course. Other administration schedules may also be deemed appropriate using sound medical judgement

The dosing can be continuous (7 days of administration in a week) or intermittent, for example, depending on the pharmacokinetics and a particular subject's clearance/accumulation of the drug. If intermittently, the schedule may be, for example, 4 days of administration and 3 days off (rest days) in a week or any other intermittent dosing schedule deemed appropriate using sound medical judgement. For example, intermittent dosing may involve administration of a single dose within a treatment course. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

In some embodiments, the use of compositions of the disclosure may be used as a standalone therapy. In some embodiments, the use of compositions of the disclosure may be used as an adjuvant/combination therapy. In some embodiments, the additional therapy is an antidepressant, an anticonvulsant, lisdexamfetamine dimesylate, an antipsychotic, an anxiolytic, an anti-inflammatory drug, a benzodiazepine, an analgesic drug, a cardiovascular drug, an opioid antagonist, psychotherapy, or combinations thereof.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the compounds Formula (I) through (V)), and yet is entirely effective to treat the clinical symptoms demonstrated by the particular patient. This planning should involve the careful choice of active ingredient by considering factors such as compound potency, relative bioavailability, patient body weight, presence and severity of adverse side effects, preferred mode of administration, and the toxicity profile of the selected agent.

A therapeutically effective dose may vary depending on the variety of factors described above, but is typically that which provides the compound of Formula (I) through (V) in an amount of about 0.00001 mg to about 10 mg per kilogram body weight of the recipient, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg of the compound of Formula (I) through (V)(on an active basis).

The compounds of the present disclosure (e.g., a compound of Formula (I) through (V)) may be administered at a psychedelic dose. Psychedelic dosing, by mouth or otherwise, may in some embodiments range from about 0.083 mg/kg, about 0.09 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, and up to about 5 mg/kg, about 4 mg/kg, about 3 mg/kg, about 2 mg/kg, about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg of the compound of Formula (I) through (V)(on an active basis). Higher dosing may also be used in some embodiments, as described above. In some embodiments, psychedelic doses are administered once by mouth, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose. These treatments are appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

The compounds of the present disclosure (e.g., a compound of Formula (I) through (V)), may be administered at sub-psychedelic/sub-psychoactive (yet still potentially serotonergic) concentrations to achieve durable therapeutic benefits, with decreased toxicity, and may thus be suitable for microdosing. Sub-psychedelic dosing, by mouth or otherwise, may in some embodiments range from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and less than about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the compound of Formula (I) through (V)(on an active basis). Typically, sub-psychedelic doses are administered orally up to every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday.

Sub-psychedelic dosing can also be carried out, for example, by transdermal delivery, subcutaneous administration, etc., via modified, controlled, slow, or extended release dosage forms, including, but not limited to, depot dosage forms, implants, patches, and pumps, which can be optionally remotely controlled. Here, doses would achieve similar blood levels as low oral dosing, but would nevertheless be sub-psychedelic.

Sub-psychedelic doses can be used, e.g., for the chronic treatment a variety of diseases or disorders disclosed herein, examples of which include, but are not limited to, inflammation, pain, and neuroinflammation.

The compounds of the present disclosure (e.g., a compound of Formula (I) through (V)), may be used for a maintenance regimen. As used herein, a “maintenance regimen” generally refers to the administration of the compounds of the present disclosure (e.g., a compound of Formula (I) through (V)) following achievement of a target dose, e.g., following completion of an up-titration regimen, and/or following a positive clinical response, e.g., improvement of the patient's condition, either to the same drug or to a different drug. In some embodiments, the patient is administered a first drug for a therapeutic regimen and a second drug for a maintenance regimen, wherein the first and second drugs are different. For example, the patient may be administered a therapeutic regimen of a first drug which is not a compound of the present disclosure (e.g., the first drug is a serotonergic psychedelic such as LSD, psilocybin, MDMA, dimethyltryptamine, etc., or a non-psychedelic drug), followed by a compound of the present disclosure (as the second drug) in a maintenance regimen. In another example, a different compound of the present disclosure is used for the therapeutic regimen (first drug) than is used for the maintenance regimen (second drug). In some embodiments, the patient is administered the same compound of the present disclosure for both a therapeutic regimen and a maintenance regimen. In any case, the maintenance dose of the compounds of the present disclosure may be used to ‘maintain’ the therapeutic response and/or to prevent occurrences of relapse. When the same compound of the present disclosure is used for both the original therapeutic regimen and for the maintenance regimen, the maintenance dose of the compound may be at or below the therapeutic dose. In some embodiments, the maintenance dose is a psychedelic dose. In some embodiments, the maintenance dose is a sub-psychedelic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

The subjects treated herein may have a disease or disorder associated with a serotonin 5-HT₂ receptor.

In some embodiments, the disease or disorder is a neuropsychiatric disease or disorder or an inflammatory disease or disorder. In some embodiments, the neuropsychiatric disease or disorder is not schizophrenia or cognitive deficits in schizophrenia.

In some embodiments, the disease or disorder is a central nervous system (CNS) disorder, including, but not limited to, major depressive disorder (MDD), treatment-resistant depression (TRD), post-traumatic stress disorder (PTSD), bipolar and related disorders (including, but not limited to, bipolar I disorder, bipolar II disorder, cyclothymic disorder), obsessive-compulsive disorder (OCD), generalized anxiety disorder (GAD), social anxiety disorder, substance use disorders (including, but not limited to, alcohol use disorder, opioid use disorder, amphetamine use disorder, nicotine use disorder, smoking, and cocaine use disorder), eating disorders (including, but not limited to anorexia nervosa, bulimia nervosa, binge-eating disorder, etc.), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido, hypoactive sexual desire disorder (HSDD), etc.), peripheral neuropathy, and obesity.

In some embodiments, the disease or disorder is major depressive disorder (MDD).

In some embodiments, the disease or disorder is treatment-resistant depression (TRD).

In some embodiments, the disease or disorder is an anxiety-related disorder, such as generalized anxiety disorder (GAD), social anxiety disorder, panic disorder, a phobia-related disorder (e.g., phobias related to flying, heights, specific animals such as spiders/dogs/snakes, receiving injections, blood, etc., agoraphobia), separation anxiety disorder, selective mutism, etc. In some embodiments, the disease or disorder is generalized anxiety disorder (GAD). In some embodiments, the disease or disorder is social anxiety disorder.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, etc. In some embodiments, the disease or disorder is obsessive-compulsive disorder (OCD).

In some embodiments, the disease or disorder is headaches (e.g., cluster headache, migraine, etc.).

In some embodiments, the disease or disorder is a substance use disorder. In some embodiments, the disease or disorder is alcohol use disorder. In some embodiments, the disease or disorder is nicotine use (e.g., smoking) disorder, and the therapy is used for e.g., smoking cessation.

In some embodiments, the disease or disorder is a disease or disorder characterized by, or otherwise associated with, neuroinflammation. Compounds and compositions of the present disclosure may provide cognitive benefits to subject's suffering from neurological and neurodegenerative diseases such as Alzheimer's disease and other dementia subtypes, Parkinson's disease, and others where neuroinflammation is a hallmark of disease pathophysiology and progression. For example, emerging psychedelic research/clinical evidence indicates that psychedelics, including tryptamine psychedelics (e.g., psilocybin), may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the compounds of the present disclosure (e.g., a compound of Formula (I) through (V)) are used for the treatment of neurological and neurodegenerative disorders such as Alzheimer's disease, dementia subtypes, and Parkinson's disease, where neuroinflammation is associated with disease pathogenesis. In some embodiments, the compounds of the present disclosure are used for the treatment of Alzheimer's disease. In some embodiments, the compounds of the present disclosure are used for the treatment of dementia. In some embodiments, the compounds of the present disclosure are used for the treatment of Parkinson's disease. As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical compositions adapted for oral and/or extended-release dosing are appropriate for such treatment methods, with sub-psychedelic dosing being preferred.

Further, many of the behavioral issues associated with chronic and/or life-threatening illnesses, including neurodegenerative disorders such as Alzheimer's disease, may benefit from treatment with the compounds disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Compounds described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder. In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is cancer related depression and anxiety. As discussed above, oral and/or extended-release dosing is appropriate for such applications, particularly when blood concentrations of active ingredient (e.g., a compound of Formula (I) through (V)) are kept below the psychedelic threshold.

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism

Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the disease or disorder includes conditions of the autonomic nervous system (ANS).

In some embodiments, the disease or disorder includes pulmonary disorders including asthma and chronic obstructive pulmonary disorder (COPD).

In some embodiments, the disease or disorder includes cardiovascular disorders including atherosclerosis.

In some embodiments, the disclosure relates to a method of treating an ocular disease, such as uveitis, corneal disease, iritis, iridocyclitis, glaucoma, and cataracts, by administering ophthalmically a therapeutically effective amount of any of the compounds described herein to a subject in need thereof. For example, compounds herein may be administered in the form of solutions, suspensions, ointments, emulsions, gel-forming solutions, powders for solutions, gels, ocular inserts, and implants. In some embodiments, the compounds are administered in the form of an eye drop formulation.

The administering physician can provide a method of treatment that is prophylactic or therapeutic by adjusting the amount and timing of any of the compounds/forms described herein on the basis of observations of one or more symptoms of the disorder or condition being treated. In some embodiments, the subject is a mammal. In some embodiments, the mammal is a human.

Also disclosed herein is a method of increasing oral bioavailability relative to DMT, 5-MeO-DMT, psilocybin, and/or psilocin, comprising administering a therapeutically effective amount of a compound as disclosed herein (e.g., a compound of Formula (I) through (V), or a pharmaceutically acceptable salt, stereoisomer, tautomer, solvate, polymorph, or prodrug thereof) to a patient in need thereof. For example, use of a compound of Formula (II) may increase oral bioavailability relative to DMT. In another example, use of a compound of

Formula (III) may increase oral bioavailability relative to psilocin. In yet another example, use of a compound of Formula (IV) may increase oral bioavailability relative to 5-MeO-DMT. In yet another example, use of a compound of Formula (V) may increase oral bioavailability relative to psilocybin.

Also disclosed herein is a method of reducing psychedelic side effects relative to DMT, 5-MeO-DMT, psilocybin, and/or psilocin, comprising administering a therapeutically effective amount of a compound as disclosed herein (i.e., a compound of Formula (I) through (V), or a pharmaceutically acceptable salt, stereoisomer, tautomer, solvate, polymorph, or prodrug thereof) to a patient in need thereof.

The terms “hallucinogenic side effects” and “psychedelic side effects” are used in the present disclosure interchangeably to refer to unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other substantial subjective changes in cognition, memory, emotion and consciousness.

In some embodiments, the administration of the compounds of the present disclosure causes no hallucinogenic and/or psychedelic side effects and/or less hallucinogenic and/or psychedelic side effects relative to DMT, 5-MeO-DMT, psilocybin, and/or psilocin. In some embodiments, the administration of the compound of Formula (I) through (V), or a pharmaceutically acceptable salt, stereoisomer, tautomer, solvate, polymorph, or prodrug thereof, alleviates, reduces, removes, and/or eliminates the hallucinogenic and/or psychedelic side effects compared to those caused by administration of DMT, 5-MeO-DMT, psilocybin, and/or psilocin.

Also disclosed herein is a method of reducing dose related side-effects, e.g., nausea, relative to treatment with DMT, 5-MeO-DMT, psilocybin, and/or psilocin, comprising administering a therapeutically effective amount of a compound as disclosed herein (e.g., the compound of Formula (I) through (V), or a pharmaceutically acceptable salt, stereoisomer, tautomer, solvate, polymorph, or prodrug thereof) to a subject in need thereof. The compound of Formula (I) through (V) may provide better brain penetration (i.e., a higher brain: plasma ratio) than that obtained from administration of DMT, 5-MeO-DMT, psilocybin, and/or psilocin. As a result, the effective dosing for the compounds of the present disclosure can be lowered, thereby reducing dose related side effects such as nausea.

Also disclosed herein is a method of increasing duration of therapeutic effect relative to DMT, 5-MeO-DMT, and/or psilocin, comprising administering a therapeutically effective amount of a compound as disclosed herein to a patient in need thereof. For example, use of a compound of Formula (II) may increase duration of action relative to DMT. In another example, use of a compound of Formula (III) may increase duration of action relative to psilocin. In yet another example, use of a compound of Formula (IV) may increase duration of action relative to 5-MeO-DMT.

### EXAMPLES

### I. Synthetic routes

Compounds of the present disclosure may generally be prepared according to, or analogous to, the following synthetic procedures, which are depicted in FIGS. 1-21.

### Example 1

### N,N-dimethyl-2-(5-((trifluoromethyl)thio)-1H-indol-3-yl) ethan-1-amine (I-3)

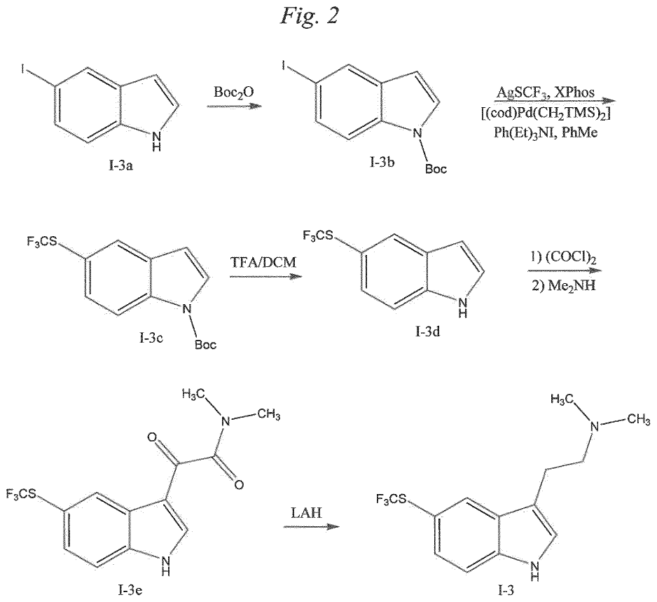

Synthesis of N,N-dimethyl-2-(5-((trifluoromethyl)thio)-1H-indol-3-yl) ethan-1-amine (I-3) was carried out according to FIG. 2. 5-iodo-1H-indole (I-3a) was reacted with Boc₂O (1.2 eq.) and cat. DMAP to provide intermediate I-3b in quantitative yield. A Pd/XPhos-catalyzed cross coupling was performed by reacting intermediate I-3b with AgSCF₃ (1.3 eq.) using (1,5-cyclooctadiene)bis(trimethylsilylmethyl) palladium (II) catalyst (0.14 eq.) in the presence of XPhos (0.17 eq.) and phenyltriethylammonium iodide (1.3 eq.) in toluene at 85-90° C. for 1.5 h to provide intermediate I-3c in 55% yield. Boc removal with TFA/DCM (1/1) for 2 h at room temperature, followed by a basic aqueous wash then provided intermediate I-3d in quantitative yield. Intermediate I-3d was reacted with oxalyl chloride (4 eq.) at 0° C. to room temperature overnight, followed by addition of dimethylamine (10 eq.) for 30 minutes at room temperature to provide intermediate I-3e in 71% yield. Reduction with LAH (5 eq.) in dioxane at 85° C. provided crude product residue which was purified by flash column chromatography to provide the title compound in 13% yield; the structure was confirmed by ¹H NMR, ¹⁹F-NMR, MS, and LC.

### Example 2

### N-(2-(1H-indol-3-yl)ethyl)-N-(3,3-difluoropropyl)-3,3-difluoropropan-1-amine (II-2)

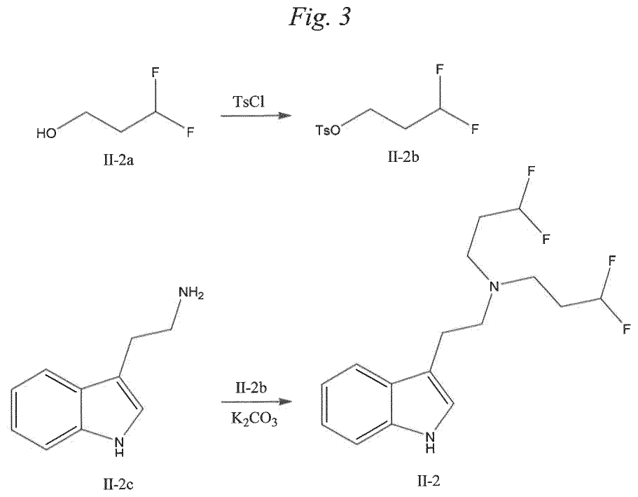

Synthesis of N-(2-(1H-indol-3-yl)ethyl)-N-(3,3-difluoropropyl)-3,3-difluoropropan-1-amine (II-2) was carried out according to FIG. 3. 3,3-difluoropropan-1-ol (II-2a) was reacted with tosyl chloride (1.5 eq.) in the presence of triethylamine (2 eq.) and cat. DMAP in DCM overnight to provide crude residue which was purified by flash column chromatography to give intermediate II-2b in 63% yield. 2-(1H-indol-3-yl) ethan-1-amine (II-2c) was then reacted with intermediate II-2b (2.1 eq.) in acetonitrile and K₂CO₃ (2.1 eq.) at 70-80° C. to provide crude product residue which was purified by flash column chromatography to provide the title compound in 7% yield.

### Example 3

### N-(2-(1H-indol-3-yl)ethyl)-3,3,3-trifluoro-N-(3,3,3-trifluoropropyl) propan-1-amine (II-3)

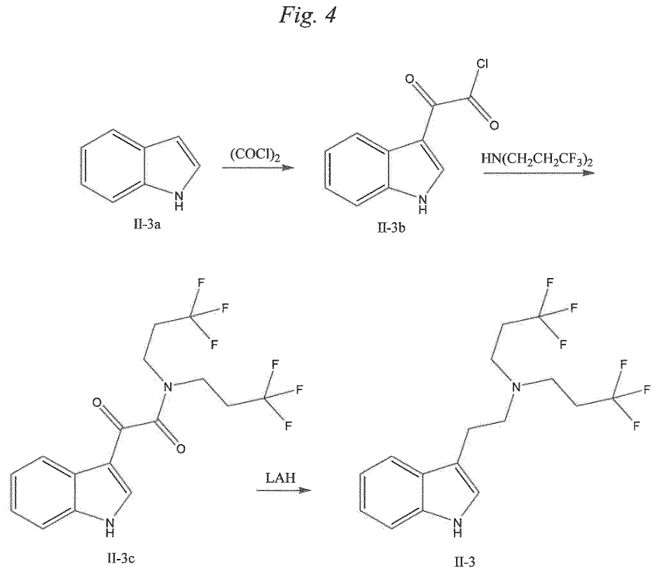

Synthesis of N-(2-(1H-indol-3-yl)ethyl)-3,3,3-trifluoro-N-(3,3,3-trifluoropropyl) propan-1-amine (II-3) was carried out according to FIG. 4. Indole (II-3a) was reacted with oxalyl chloride (4 eq.) at room temperature for 1.5 h to provide intermediate II-3b in 74% yield. Intermediate II-3b was reacted with bis(3,3,3-trifluoropropyl)amine (2 eq.) for 2 h at room temperature to provide intermediate II-3c in quantitative yield. Reduction with LAH (6 eq.) in dioxane at 90° C. overnight provided crude product residue which was purified by flash column chromatography to provide the title compound in 50% yield; the structure was confirmed by ¹H NMR, MS, and LC.

### Example 4

### N-(2-(1H-indol-3-yl)ethyl)-3,3,3-trifluoro-N-methylpropan-1-amine (II-6)

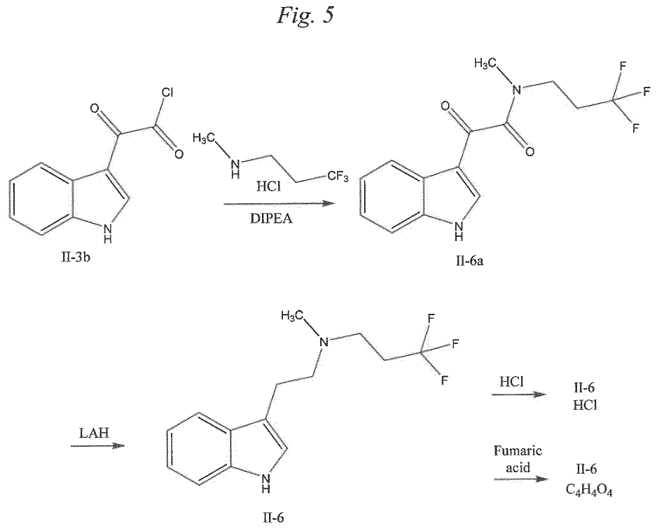

Synthesis of N-(2-(1H-indol-3-yl)ethyl)-3,3,3-trifluoro-N-methylpropan-1-amine (II-6) was carried out according to FIG. 5. II-3b was reacted with 3,3,3-trifluoro-N-methylpropan-1-amine-HCl salt (3 eq.) in the presence of DIPEA (6 eq.) overnight at room temperature to provide intermediate II-6a in 85% yield. Reduction with LAH (6 eq.) in dioxane at 85-90° C. overnight provided crude product residue which was purified by flash column chromatography to provide the title compound in 12% yield; the structure was confirmed by ¹H NMR and LC.

The HCl salt of II-6 was prepared by dissolving II-6 (free base) prepared above in methanol followed by treatment with HCl in diethyl ether (4 eq. HCl).

The fumarate salt of II-6 was prepared by dissolving II-6 (free base) prepared above in methanol followed by treatment with fumaric acid (1 eq.).

### Example 5

### N-(2-(1H-indol-3-yl)ethyl)-2,2,2-trifluoro-N-(2,2,2-trifluoroethyl) ethan-1-amine (II-10)

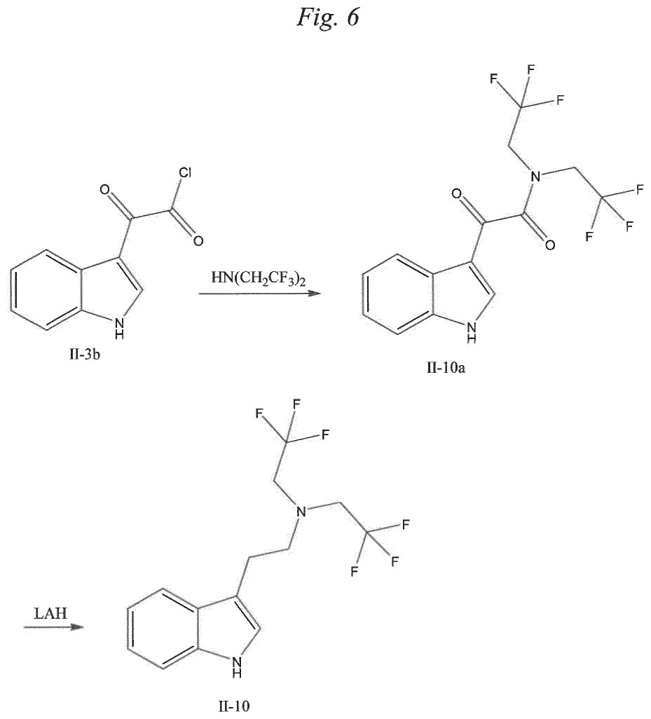

Synthesis of N-(2-(1H-indol-3-yl)ethyl)-2,2,2-trifluoro-N-(2,2,2-trifluoroethyl) ethan-1-amine (II-10) was carried out according to FIG. 6. II-3b was reacted with bis(2,2,2-trifluoroethyl)amine (3 eq.) in THF from 0° C. to room temperature over 2.5 h to provide intermediate II-10a in 85% yield. Reduction with LAH (6.4 eq.) in dioxane at 80° C. for 90 min provided crude product residue which was purified by flash column chromatography to provide the title compound in 19% yield; the structure was confirmed by ¹H NMR and MS.

Typically, sub-psychedelic doses are administered orally up to every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday.

Sub-psychedelic dosing can also be carried out, for example, by transdermal delivery, subcutaneous administration, etc., via modified, controlled, slow, or extended release dosage forms, including, but not limited to, depot dosage forms, implants, patches, and pumps, which can be optionally remotely controlled. Here, doses would achieve similar blood levels as low oral dosing, but would nevertheless be sub-psychedelic.

Sub-psychedelic doses can be used, e.g., for the chronic treatment a variety of diseases or disorders disclosed herein, examples of which include, but are not limited to, inflammation, pain, and neuroinflammation.

The compounds of the present disclosure (e.g., a compound of Formula (I) through (V)), may be used for a maintenance regimen. As used herein, a “maintenance regimen” generally refers to the administration of the compounds of the present disclosure (e.g., a compound of Formula (I) through (V)) following achievement of a target dose, e.g., following completion of an up-titration regimen, and/or following a positive clinical response, e.g., improvement of the patient's condition, either to the same drug or to a different drug. In some embodiments, the patient is administered a first drug for a therapeutic regimen and a second drug for a maintenance regimen, wherein the first and second drugs are different. For example, the patient may be administered a therapeutic regimen of a first drug which is not a compound of the present disclosure (e.g., the first drug is a serotonergic psychedelic such as LSD, psilocybin, MDMA, dimethyltryptamine, etc., or a non-psychedelic drug), followed by a compound of the present disclosure (as the second drug) in a maintenance regimen. In another example, a different compound of the present disclosure is used for the therapeutic regimen (first drug) than is used for the maintenance regimen (second drug). In some embodiments, the patient is administered the same compound of the present disclosure for both a therapeutic regimen and a maintenance regimen. In any case, the maintenance dose of the compounds of the present disclosure may be used to ‘maintain’ the therapeutic response and/or to prevent occurrences of relapse. When the same compound of the present disclosure is used for both the original therapeutic regimen and for the maintenance regimen, the maintenance dose of the compound may be at or below the therapeutic dose. In some embodiments, the maintenance dose is a psychedelic dose. In some embodiments, the maintenance dose is a sub-psychedelic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

The subjects treated herein may have a disease or disorder associated with a serotonin 5-HT₂ receptor.

In some embodiments, the disease or disorder is a neuropsychiatric disease or disorder or an inflammatory disease or disorder. In some embodiments, the neuropsychiatric disease or disorder is not schizophrenia or cognitive deficits in schizophrenia.

In some embodiments, the disease or disorder is a central nervous system (CNS) disorder, including, but not limited to, major depressive disorder (MDD), treatment-resistant depression (TRD), post-traumatic stress disorder (PTSD), bipolar and related disorders (including, but not limited to, bipolar I disorder, bipolar II disorder, cyclothymic disorder), obsessive-compulsive disorder (OCD), generalized anxiety disorder (GAD), social anxiety disorder, substance use disorders (including, but not limited to, alcohol use disorder, opioid use disorder, amphetamine use disorder, nicotine use disorder, smoking, and cocaine use disorder), eating disorders (including, but not limited to anorexia nervosa, bulimia nervosa, binge-eating disorder, etc.), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido, hypoactive sexual desire disorder (HSDD), etc.), peripheral neuropathy, and obesity.

In some embodiments, the disease or disorder is major depressive disorder (MDD).

In some embodiments, the disease or disorder is treatment-resistant depression (TRD).

In some embodiments, the disease or disorder is an anxiety-related disorder, such as generalized anxiety disorder (GAD), social anxiety disorder, panic disorder, a phobia-related disorder (e.g., phobias related to flying, heights, specific animals such as spiders/dogs/snakes, receiving injections, blood, etc., agoraphobia), separation anxiety disorder, selective mutism, etc. In some embodiments, the disease or disorder is generalized anxiety disorder (GAD). In some embodiments, the disease or disorder is social anxiety disorder.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, etc. In some embodiments, the disease or disorder is obsessive-compulsive disorder (OCD).

In some embodiments, the disease or disorder is headaches (e.g., cluster headache, migraine, etc.).

In some embodiments, the disease or disorder is a substance use disorder. In some embodiments, the disease or disorder is alcohol use disorder. In some embodiments, the disease or disorder is nicotine use (e.g., smoking) disorder, and the therapy is used for e.g., smoking cessation.

In some embodiments, the disease or disorder is a disease or disorder characterized by, or otherwise associated with, neuroinflammation. Compounds and compositions of the present disclosure may provide cognitive benefits to subject's suffering from neurological and neurodegenerative diseases such as Alzheimer's disease and other dementia subtypes, Parkinson's disease, and others where neuroinflammation is a hallmark of disease pathophysiology and progression. For example, emerging psychedelic research/clinical evidence indicates that psychedelics, including tryptamine psychedelics (e.g., psilocybin), may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the compounds of the present disclosure (e.g., a compound of Formula (I) through (V)) are used for the treatment of neurological and neurodegenerative disorders such as Alzheimer's disease, dementia subtypes, and Parkinson's disease, where neuroinflammation is associated with disease pathogenesis. In some embodiments, the compounds of the present disclosure are used for the treatment of Alzheimer's disease. In some embodiments, the compounds of the present disclosure are used for the treatment of dementia. In some embodiments, the compounds of the present disclosure are used for the treatment of Parkinson's disease. As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical compositions adapted for oral and/or extended-release dosing are appropriate for such treatment methods, with sub-psychedelic dosing being preferred.

Further, many of the behavioral issues associated with chronic and/or life-threatening illnesses, including neurodegenerative disorders such as Alzheimer's disease, may benefit from treatment with the compounds disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Compounds described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder. In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is cancer related depression and anxiety. As discussed above, oral and/or extended-release dosing is appropriate for such applications, particularly when blood concentrations of active ingredient (e.g., a compound of Formula (I) through (V)) are kept below the psychedelic threshold.

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the disease or disorder includes conditions of the autonomic nervous system (ANS).

In some embodiments, the disease or disorder includes pulmonary disorders including asthma and chronic obstructive pulmonary disorder (COPD).

In some embodiments, the disease or disorder includes cardiovascular disorders including atherosclerosis.

In some embodiments, the disclosure relates to a method of treating an ocular disease, such as uveitis, corneal disease, iritis, iridocyclitis, glaucoma, and cataracts, by administering ophthalmically a therapeutically effective amount of any of the compounds described herein to a subject in need thereof. For example, compounds herein may be administered in the form of solutions, suspensions, ointments, emulsions, gel-forming solutions, powders for solutions, gels, ocular inserts, and implants. In some embodiments, the compounds are administered in the form of an eye drop formulation.

The administering physician can provide a method of treatment that is prophylactic or therapeutic by adjusting the amount and timing of any of the compounds/forms described herein on the basis of observations of one or more symptoms of the disorder or condition being treated. In some embodiments, the subject is a mammal. In some embodiments, the mammal is a human.

Also disclosed herein is a method of increasing oral bioavailability relative to DMT, 5-MeO-DMT, psilocybin, and/or psilocin, comprising administering a therapeutically effective amount of a compound as disclosed herein (e.g., a compound of Formula (I) through (V), or a pharmaceutically acceptable salt, stereoisomer, tautomer, solvate, polymorph, or prodrug thereof) to a patient in need thereof. For example, use of a compound of Formula (II) may increase oral bioavailability relative to DMT. In another example, use of a compound of Formula (III) may increase oral bioavailability relative to psilocin. In yet another example, use of a compound of

Formula (IV) may increase oral bioavailability relative to 5-MeO-DMT. In yet another example, use of a compound of Formula (V) may increase oral bioavailability relative to psilocybin.

Also disclosed herein is a method of reducing psychedelic side effects relative to DMT, 5-MeO-DMT, psilocybin, and/or psilocin, comprising administering a therapeutically effective amount of a compound as disclosed herein (i.e., a compound of Formula (I) through (V), or a pharmaceutically acceptable salt, stereoisomer, tautomer, solvate, polymorph, or prodrug thereof) to a patient in need thereof.

The terms “hallucinogenic side effects” and “psychedelic side effects” are used in the present disclosure interchangeably to refer to unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other substantial subjective changes in cognition, memory, emotion and consciousness.

In some embodiments, the administration of the compounds of the present disclosure causes no hallucinogenic and/or psychedelic side effects and/or less hallucinogenic and/or psychedelic side effects relative to DMT, 5-MeO-DMT, psilocybin, and/or psilocin. In some embodiments, the administration of the compound of Formula (I) through (V), or a pharmaceutically acceptable salt, stereoisomer, tautomer, solvate, polymorph, or prodrug thereof, alleviates, reduces, removes, and/or eliminates the hallucinogenic and/or psychedelic side effects compared to those caused by administration of DMT, 5-MeO-DMT, psilocybin, and/or psilocin. Also disclosed herein is a method of reducing dose related side-effects, e.g., nausea, relative to treatment with DMT, 5-MeO-DMT, psilocybin, and/or psilocin, comprising administering a therapeutically effective amount of a compound as disclosed herein (e.g., the compound of Formula (I) through (V), or a pharmaceutically acceptable salt, stereoisomer, tautomer, solvate, polymorph, or prodrug thereof) to a subject in need thereof. The compound of Formula (I) through (V) may provide better brain penetration (i.e., a higher brain: plasma ratio) than that obtained from administration of DMT, 5-MeO-DMT, psilocybin, and/or psilocin. As a result, the effective dosing for the compounds of the present disclosure can be lowered, thereby reducing dose related side effects such as nausea.

Also disclosed herein is a method of increasing duration of therapeutic effect relative to DMT, 5-MeO-DMT, and/or psilocin, comprising administering a therapeutically effective amount of a compound as disclosed herein to a patient in need thereof. For example, use of a compound of Formula (II) may increase duration of action relative to DMT. In another example, use of a compound of Formula (III) may increase duration of action relative to psilocin. In yet another example, use of a compound of Formula (IV) may increase duration of action relative to 5-MeO-DMT.

### EXAMPLES

### I. Synthetic routes

Compounds of the present disclosure may generally be prepared according to, or analogous to, the following synthetic procedures, which are depicted in FIGS. 1-21.

### Example 1

### N,N-dimethyl-2-(5-((trifluoromethyl)thio)-1H-indol-3-yl) ethan-1-amine (I-3)

Synthesis of N,N-dimethyl-2-(5-((trifluoromethyl)thio)-1H-indol-3-yl) ethan-1-amine (I-3) was carried out according to FIG. 2. 5-iodo-1H-indole (I-3a) was reacted with Boc₂O (1.2 eq.) and cat. DMAP to provide intermediate I-3b in quantitative yield. A Pd/XPhos-catalyzed cross coupling was performed by reacting intermediate I-3b with AgSCF₃ (1.3 eq.) using (1,5-cyclooctadiene)bis(trimethylsilylmethyl) palladium (II) catalyst (0.14 eq.) in the presence of XPhos (0.17 eq.) and phenyltriethylammonium iodide (1.3 eq.) in toluene at 85-90° C. for 1.5 h to provide intermediate I-3c in 55% yield. Boc removal with TFA/DCM (1/1) for 2 h at room temperature, followed by a basic aqueous wash then provided intermediate I-3d in quantitative yield. Intermediate I-3d was reacted with oxalyl chloride (4 eq.) at 0° C. to room temperature overnight, followed by addition of dimethylamine (10 eq.) for 30 minutes at room temperature to provide intermediate I-3e in 71% yield. Reduction with LAH (5 eq.) in dioxane at 85° C. provided crude product residue which was purified by flash column chromatography to provide the title compound in 13% yield; the structure was confirmed by ¹H NMR, ¹⁹F-NMR, MS, and LC.

### Example 2

### N-(2-(1H-indol-3-yl)ethyl)-N-(3,3-difluoropropyl)-3,3-difluoropropan-1-amine (II-2)

Synthesis of N-(2-(1H-indol-3-yl)ethyl)-N-(3,3-difluoropropyl)-3,3-difluoropropan-1-amine (II-2) was carried out according to FIG. 3. 3,3-difluoropropan-1-ol (II-2a) was reacted with tosyl chloride (1.5 eq.) in the presence of triethylamine (2 eq.) and cat. DMAP in DCM overnight to provide crude residue which was purified by flash column chromatography to give intermediate II-2b in 63% yield. 2-(1H-indol-3-yl) ethan-1-amine (II-2c) was then reacted with intermediate II-2b (2.1 eq.) in acetonitrile and K₂CO₃ (2.1 eq.) at 70-80° C. to provide crude product residue which was purified by flash column chromatography to provide the title compound in 7% yield.

### Example 3

### N-(2-(1H-indol-3-yl)ethyl)-3,3,3-trifluoro-N-(3,3,3-trifluoropropyl) propan-1-amine (II-3)

Synthesis of N-(2-(1H-indol-3-yl)ethyl)-3,3,3-trifluoro-N-(3,3,3-trifluoropropyl) propan-1-amine (II-3) was carried out according to FIG. 4. Indole (II-3a) was reacted with oxalyl chloride (4 eq.) at room temperature for 1.5 h to provide intermediate II-3b in 74% yield. Intermediate II-3b was reacted with bis(3,3,3-trifluoropropyl)amine (2 eq.) for 2 h at room temperature to provide intermediate II-3c in quantitative yield. Reduction with LAH (6 eq.) in dioxane at 90° C. overnight provided crude product residue which was purified by flash column chromatography to provide the title compound in 50% yield; the structure was confirmed by ¹H NMR, MS, and LC.

### Example 4

### N-(2-(1H-indol-3-yl)ethyl)-3,3,3-trifluoro-N-methylpropan-1-amine (II-6)

Synthesis of N-(2-(1H-indol-3-yl)ethyl)-3,3,3-trifluoro-N-methylpropan-1-amine (II-6) was carried out according to FIG. 5. II-3b was reacted with 3,3,3-trifluoro-N-methylpropan-1-amine-HCl salt (3 eq.) in the presence of DIPEA (6 eq.) overnight at room temperature to provide intermediate II-6a in 85% yield. Reduction with LAH (6 eq.) in dioxane at 85-90° C. overnight provided crude product residue which was purified by flash column chromatography to provide the title compound in 12% yield; the structure was confirmed by ¹H NMR and LC.

The HCl salt of II-6 was prepared by dissolving II-6 (free base) prepared above in methanol followed by treatment with HCl in diethyl ether (4 eq. HCl).

The fumarate salt of II-6 was prepared by dissolving II-6 (free base) prepared above in methanol followed by treatment with fumaric acid (1 eq.).

### Example 5

### N-(2-(1H-indol-3-yl)ethyl)-2,2,2-trifluoro-N-(2,2,2-trifluoroethyl) ethan-1-amine (II-10)

Synthesis of N-(2-(1H-indol-3-yl)ethyl)-2,2,2-trifluoro-N-(2,2,2-trifluoroethyl) ethan-1-amine (II-10) was carried out according to FIG. 6. II-3b was reacted with bis(2,2,2-trifluoroethyl)amine (3 eq.) in THF from 0° C. to room temperature over 2.5 h to provide intermediate II-10a in 85% yield. Reduction with LAH (6.4 eq.) in dioxane at 80° C. for 90 min provided crude product residue which was purified by flash column chromatography to provide the title compound in 19% yield; the structure was confirmed by ¹H NMR and MS.

### Example 6

### N-(2-(1H-indol-3-yl)ethyl)-2,2-difluoro-N-methylethan-1-amine (II-12)

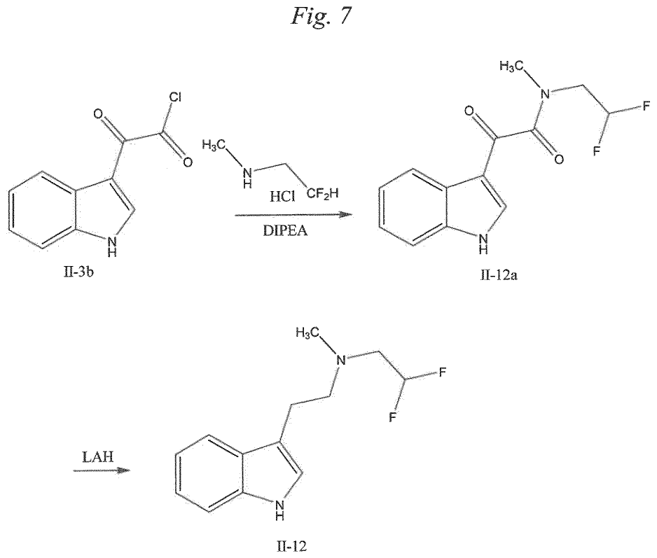

Synthesis of N-(2-(1H-indol-3-yl)ethyl)-2,2-difluoro-N-methylethan-1-amine (II-12) was carried out according to FIG. 7. II-3b was reacted with 2,2-difluoro-N-methylethan-1-amine-HCl salt (3 eq.) in the presence of DIPEA (6 eq.) overnight at room temperature to provide intermediate II-12a in 70% yield. Reduction with LAH (6 eq.) in dioxane at 85-90° C. overnight provided crude product residue which was purified by flash column chromatography to provide the title compound in 27% yield; the structure was confirmed by ¹H NMR and LC.

### Example 7

### N-(2-(1H-indol-3-yl)ethyl)-1,1,1-trifluoro-N-methylmethanesulfinamide (II-15)

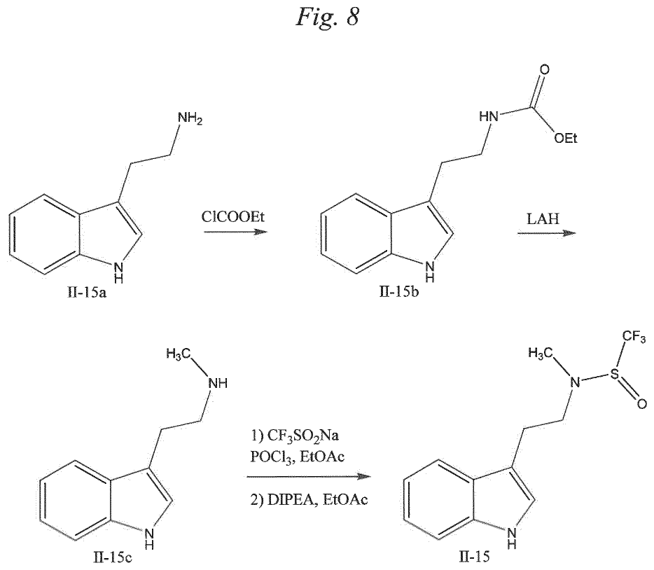

Synthesis of N-(2-(1H-indol-3-yl)ethyl)-1,1,1-trifluoro-N-methylmethanesulfinamide (II-15) was carried out according to FIG. 8. 2-(1H-indol-3-yl) ethan-1-amine (II-15a) was reacted with ethyl chloroformate (1 eq.) and sodium hydroxide (1 eq.) to provide intermediate II-15b in 87% yield. Reduction with LAH (3 eq.) in THF at 70° C. for 1 h provided intermediate II-15c in 92% yield. Next, trifluoromethanesulfinyl chloride was prepared in situ from sodium triflinate (2 eq.) and POCl₃ (1 eq.) in ethyl acetate, and after 5 min was reacted with intermediate II-15c in ethyl acetate using DIPEA (1 eq.) for 60 min at room temperature to produce crude product residue which was purified by flash column chromatography to provide the title compound in 63% yield; the structure was confirmed by ¹H NMR, LC and MS.

### Example 8

### N-(2-(1H-indol-3-yl)ethyl)-2,2,2-trifluoro-N-methylethan-1-amine (II-19)

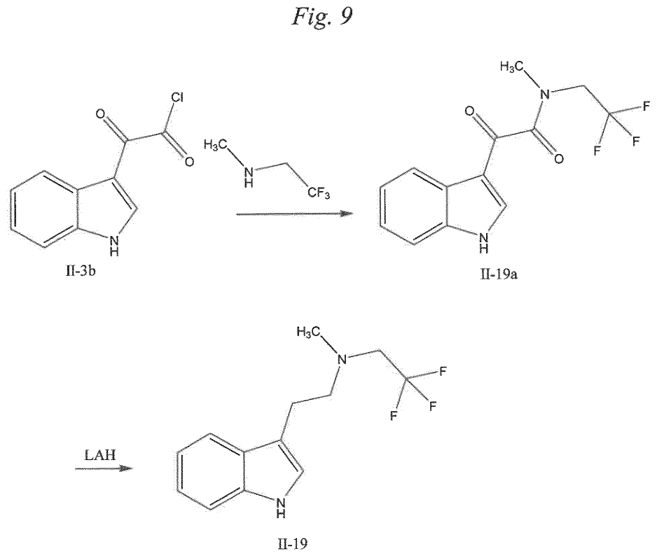

Synthesis of N-(2-(1H-indol-3-yl)ethyl)-2,2,2-trifluoro-N-methylethan-1-amine (II-19) was carried out according to FIG. 9. II-3b was reacted with 2,2,2-trifluoro-N-methylethan-1-amine in THF at 0° C. to room temperature over 2.5 h to provide intermediate II-19a in quantitative yield. Reduction with LAH (8.5 eq.) in dioxane at 80° C. for 1.5 h provided crude product residue which was purified by flash column chromatography to provide the title compound in 53% yield; the structure was confirmed by ¹H NMR and MS.

### Example 9

### N-(2-(1H-indol-3-yl)ethyl)-2,2,2-trifluoroethan-1-amine (II-20)

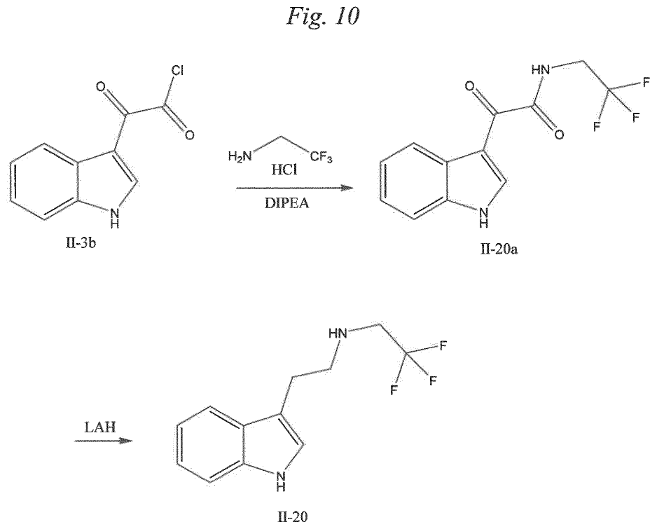

Synthesis of N-(2-(1H-indol-3-yl)ethyl)-2,2,2-trifluoroethan-1-amine (II-20) was carried out according to FIG. 10. II-3b was reacted with 2,2,2-trifluoroethan-1-amine-HCl salt in the presence of DIPEA in THF at 0° C. to room temperature over 2.5 h to provide intermediate II-20a in quantitative yield. Reduction with LAH (6.4 eq.) in dioxane at 80° C. overnight provided crude product residue which was purified by flash column chromatography to provide the title compound in 32% yield; the structure was confirmed by ¹H NMR and MS.

### Example 10

### 3-(2-(4,4-difluoropiperidin-1-yl)ethyl)-1H-indole (II-21)

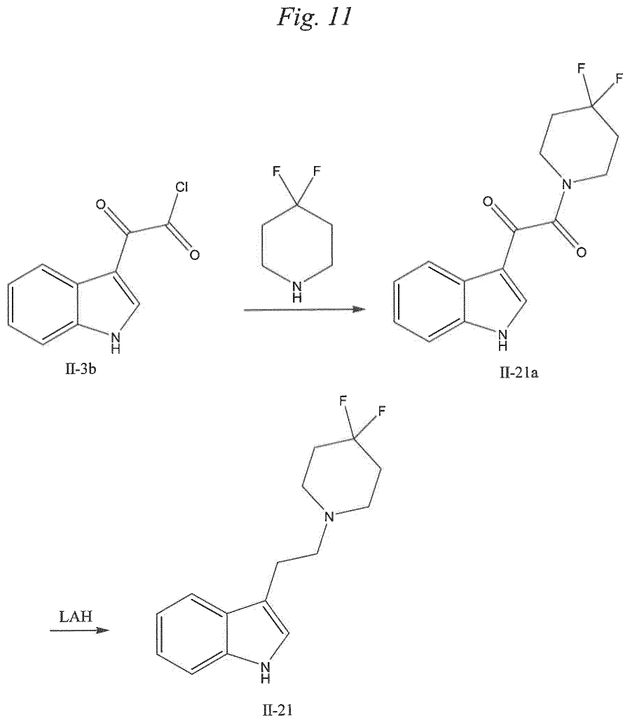

Synthesis of 3-(2-(4,4-difluoropiperidin-1-yl)ethyl)-1H-indole (II-21) was carried out according to FIG. 11. II-3b was reacted with 4,4-difluoropiperidine in DCM at room temperature for 2.5 h to provide intermediate II-21a in 56% yield. Reduction with LAH (6 eq.) in dioxane at 90° C. overnight provided crude product residue which was purified by flash column chromatography to provide the title compound in 62% yield; the structure was confirmed by ¹H NMR, LC, and MS.

### Example 11

### 3-(2-(bis(3,3-difluoropropyl)amino)ethyl)-1H-indol-4-ol (III-2)

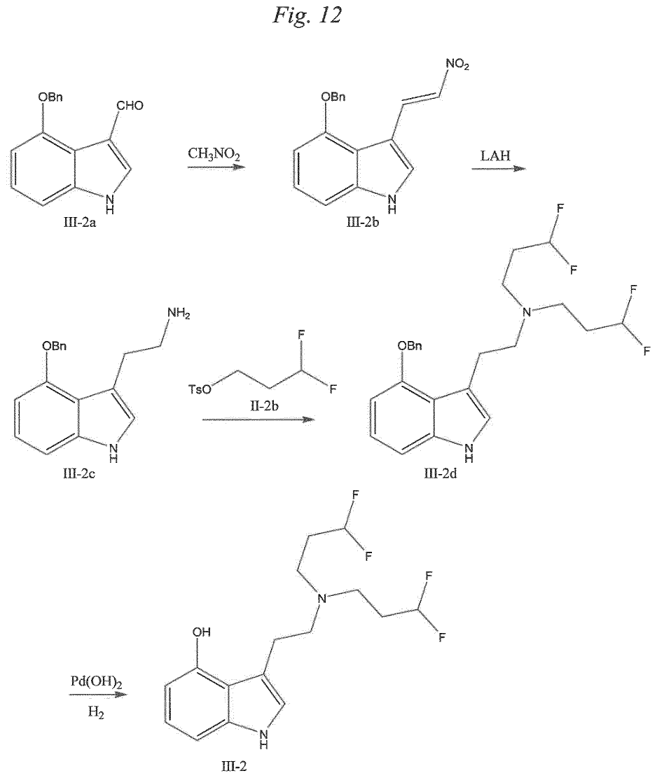

Synthesis of 3-(2-(bis(3,3-difluoropropyl)amino)ethyl)-1H-indol-4-ol (III-2) was carried out according to FIG. 12. 4-(benzyloxy)-1H-indole-3-carbaldehyde (III-2a) was reacted with nitromethane (solvent quantities) using ammonium acetate (1.1 eq.) at 90° C. for 45 min under microwave irradiation to provide intermediate III-2b in 46% yield. Per-reduction with LAH (6 eq.) in THF at 70° C. for 2.5 h provided intermediate III-2c in 64% yield. Alkylation with II-2b (2.1 eq.) in acetonitrile and K₂CO₃ (2.1 eq.) at 70-80° C. overnight, followed by a second addition of II-2b (1.1 eq.) and K₂CO₃ (1 eq.) with continued stirring at 70-80° C. overnight, followed by a third addition of II-2b (1.8 eq.) with continued stirring at 70-80° C. then provided intermediate III-2d in 75% yield. Hydrogenation with Pd(OH)₂/C and 1 atm. H₂ for 2 hr produced crude product residue which was purified by flash column chromatography to provide the title compound in 46% yield.

### Example 12

### 3-(2-(bis(3,3,3-trifluoropropyl)amino)ethyl)-1H-indol-4-ol (III-3)

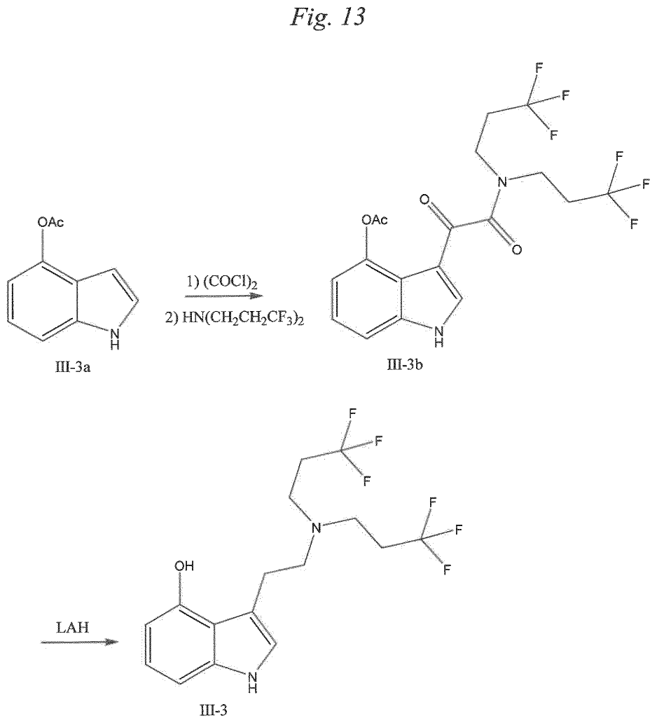

Synthesis of 3-(2-(bis(3,3,3-trifluoropropyl)amino)ethyl)-1H-indol-4-ol (III-3) was carried out according to FIG. 13. 1H-indol-4-yl acetate (III-3a) was reacted with oxalyl chloride (1.5 eq.), followed by addition of bis(3,3,3-trifluoropropyl)amine (2.77 eq.) to provide intermediate III-3b in 65% yield. Reduction with LAH (6 eq.) in dioxane at 90° C. overnight provided crude product residue which was purified by flash column chromatography to provide the title compound in 37% yield; the structure was confirmed by ¹H NMR and LC.

### Example 13

### 3-(2-(methyl (3,3,3-trifluoropropyl)amino)ethyl)-1H-indol-4-ol (III-6)

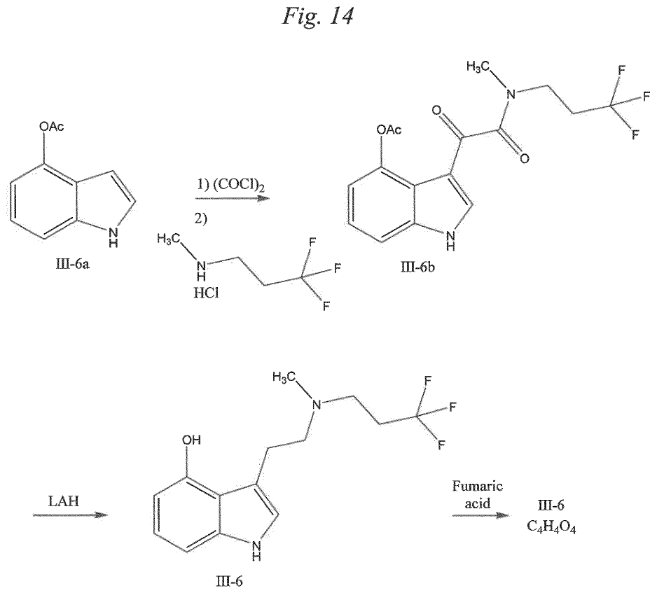

Synthesis of 3-(2-(methyl (3,3,3-trifluoropropyl)amino)ethyl)-1H-indol-4-ol (III-6) was carried out according to FIG. 14. 1H-indol-4-yl acetate (III-6a) was reacted with oxalyl chloride (1.5 eq.), followed by addition of 3,3,3-trifluoro-N-methylpropan-1-amine-HCl salt (2 eq.) and DIPEA (4 eq.) to provide intermediate III-6b in 38% yield. Reduction with LAH (6 eq.) in dioxane at 85-90° C. overnight provided crude product residue which was purified by flash column chromatography to provide the title compound in 50% yield; the structure was confirmed by LC.

The fumarate salt of III-6 was prepared by dissolving III-6 (free base) prepared above in methanol followed by treatment with fumaric acid (1 eq.).

### Example 14

### 3-(2-(bis(2,2,2-trifluoroethyl)amino)ethyl)-1H-indol-4-ol (III-10)

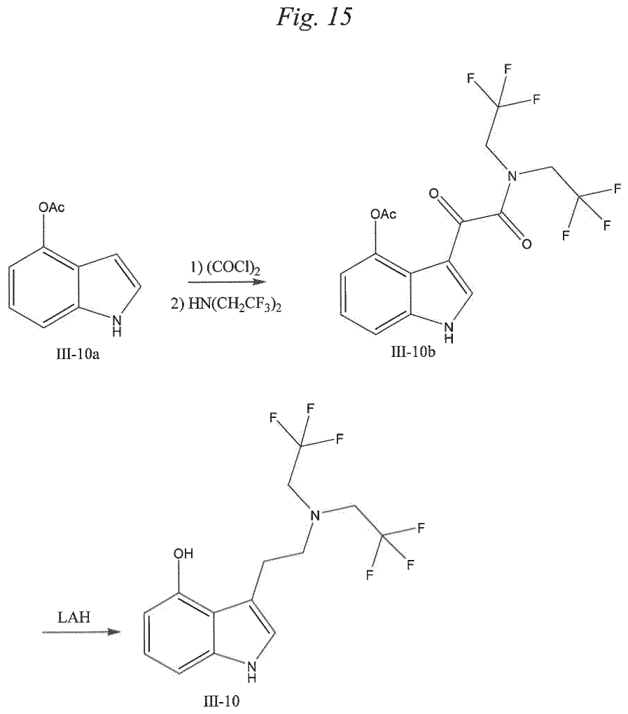

Synthesis of 3-(2-(bis(2,2,2-trifluoroethyl)amino)ethyl)-1H-indol-4-ol (III-10) was carried out according to FIG. 15. 1H-indol-4-yl acetate (III-10a) was reacted with oxalyl chloride, followed by addition of bis(2,2,2-trifluoroethyl)amine from 0° C. to room temperature to provide intermediate III-10b in 46% yield. Reduction with LAH (6 eq.) in dioxane at 85-90° C. overnight provided crude product residue which was purified by flash column chromatography to provide the title compound in 21% yield; the structure was confirmed by ¹H NMR.

### Example 15

### 3-(2-((2,2-difluoroethyl)(methyl)amino)ethyl)-1H-indol-4-ol (III-12)

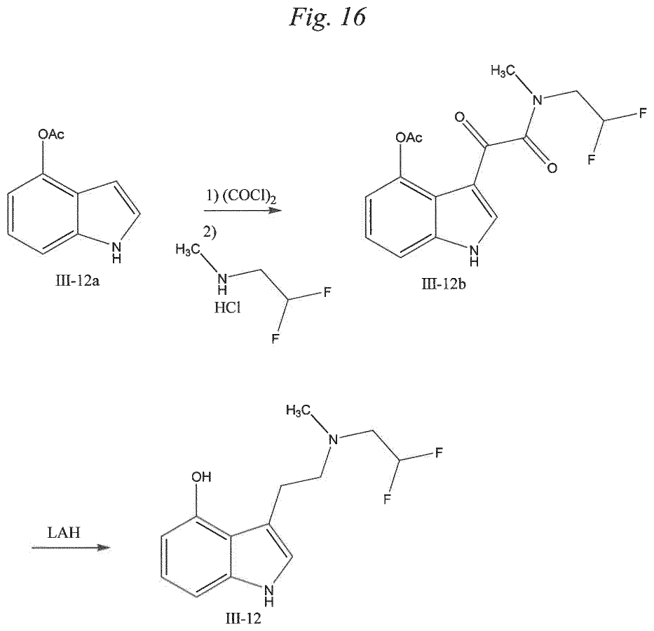

Synthesis of 3-(2-((2,2-difluoroethyl)(methyl)amino)ethyl)-1H-indol-4-ol (III-12) was carried out according to FIG. 16. 1H-indol-4-yl acetate (III-12a) was reacted with oxalyl chloride (1.5 eq.), followed by addition of 2,2-difluoro-N-methylethan-1-amine-HCl salt (2 eq.) and DIPEA (4 eq.) overnight at room temperature to provide intermediate III-12b in 38% yield. Reduction with LAH (6 eq.) in dioxane at 85-90° C. overnight provided crude product residue which was purified by flash column chromatography to provide the title compound in 45% yield; the structure was confirmed by LC.

### Example 16

### 3-(2-(methyl (2,2,2-trifluoroethyl)amino)ethyl)-1H-indol-4-ol (III-13)

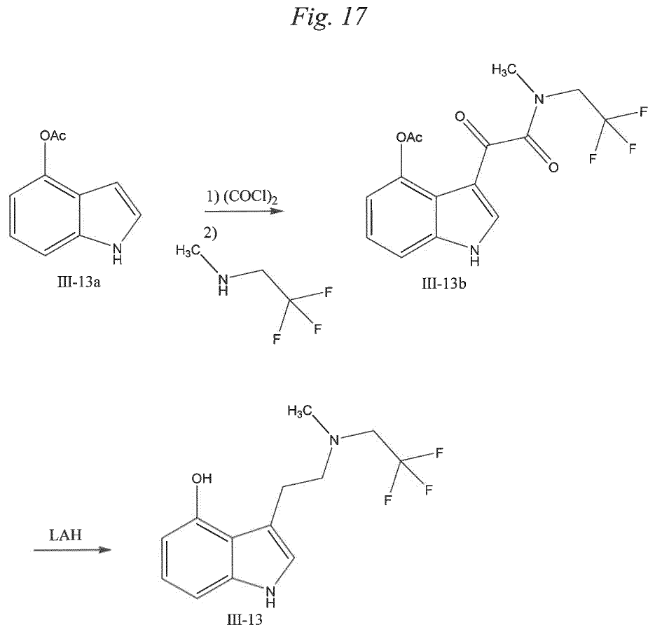

Synthesis of 3-(2-(methyl (2,2,2-trifluoroethyl)amino)ethyl)-1H-indol-4-ol (III-13) was carried out according to FIG. 17. 1H-indol-4-yl acetate (III-13a) was reacted with oxalyl chloride, followed by addition of 2,2,2-trifluoro-N-methylethan-1-amine from 0° C. to room temperature to provide intermediate III-13b in 66% yield. Reduction with LAH (6 eq.) in dioxane at 85° C. overnight provided crude product residue which was purified by flash column chromatography to provide the title compound in 50% yield; the structure was confirmed by ¹H NMR and ¹⁹F-NMR.

### Example 17

### 3-(2-((2,2,2-trifluoroethyl)amino)ethyl)-1H-indol-4-ol (III-14)

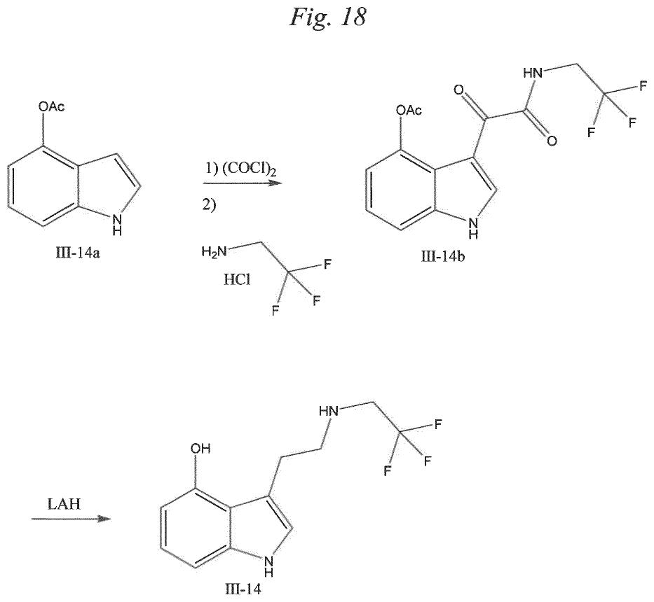

Synthesis of 3-(2-((2,2,2-trifluoroethyl)amino)ethyl)-1H-indol-4-ol (III-14) was carried out according to FIG. 18. 1H-indol-4-yl acetate (III-14a) was reacted with oxalyl chloride, followed by addition of 2,2,2-trifluoroethan-1-amine-HCl salt and DIPEA from 0° C. to room temperature to provide intermediate III-14b in 34% yield. Reduction with LAH (6 eq.) in dioxane at 85-90° C. overnight provided crude product residue which was purified by flash column chromatography to provide the title compound in 26% yield.

### Example 18

### N,N-dimethyl-2-(5-(trifluoromethoxy)-1H-indol-3-yl) ethan-1-amine (IV-3)

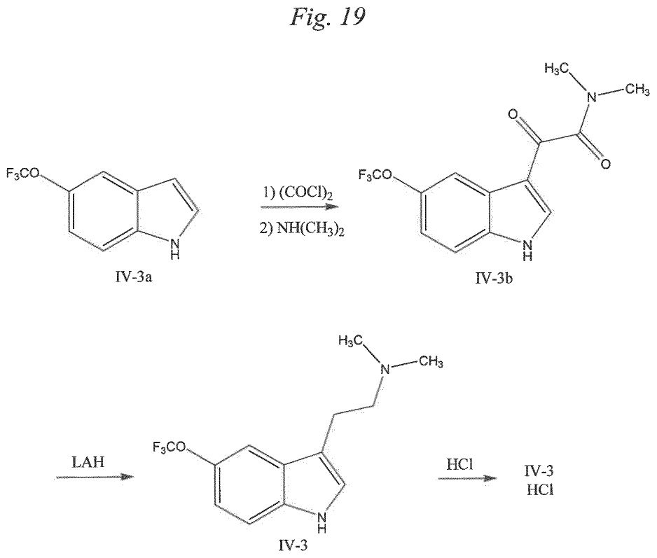

Synthesis of N,N-dimethyl-2-(5-(trifluoromethoxy)-1H-indol-3-yl) ethan-1-amine (IV-3) was carried out according to FIG. 19. 5-(trifluoromethoxy)-1H-indole (IV-3a) was reacted with oxalyl chloride, followed by addition of dimethylamine as a 2M solution in THF from 0° C. to room temperature to provide intermediate IV-3b in 24% yield. Reduction with LAH (6 eq.) in dioxane at 90° C. overnight provided crude product residue which was purified by flash column chromatography to provide the title compound in 34% yield; the structure was confirmed by ¹⁹F-NMR.

The HCl salt of IV-3 was prepared by dissolving IV-3 (free base) prepared above in methanol followed by treatment with HCl in diethyl ether.

### Example 19

### 3,3,3-trifluoro-N-(2-(5-methoxy-1H-indol-3-yl)ethyl)-N-methylpropan-1-amine (IV-21)

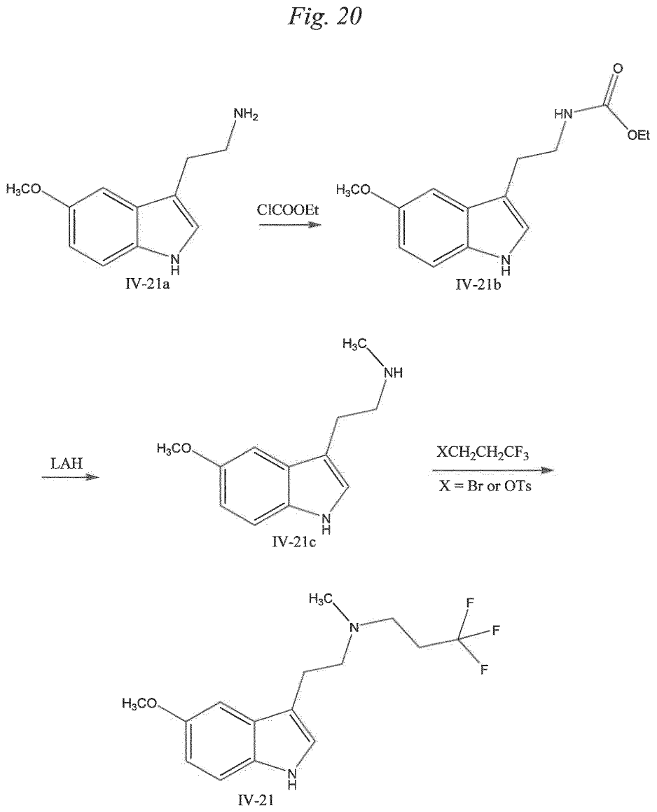

Synthesis of 3,3,3-trifluoro-N-(2-(5-methoxy-1H-indol-3-yl)ethyl)-N-methylpropan-1-amine (IV-21) was carried out according to FIG. 20. 2-(5-methoxy-1H-indol-3-yl) ethan-1-amine (IV-21a) was reacted with ethyl chloroformate (1.5 eq.) and TEA (6.8 eq.) at room temperature for 1 h to provide intermediate IV-21b in 42% yield. Reduction with LAH (4 eq.) in THF at 70° C. for 2 h provided intermediate IV-21c in 78% yield. Next, alkylation with 3-bromo-1,1,1-trifluoropropane (1.9 eq.), Nal (1 eq.), K₂CO₃ (2 eq.) in DMF at 80° C. overnight, followed by addition of 3,3,3-trifluoropropyl 4-methylbenzenesulfonate (1.5 eq.) and K₂CO₃ (3 eq.) at 80° C. overnight produced crude product residue which was purified by flash column chromatography to provide the title compound in 11% yield.

### Example 20

### 2,2,2-trifluoro-N-(2-(5-methoxy-1H-indol-3-yl)ethyl)-N-methylethan-1-amine (IV-40)

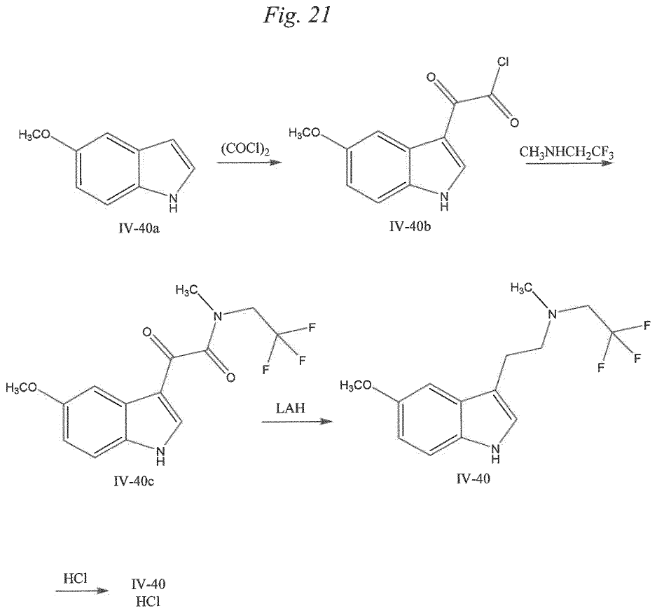

Synthesis of 2,2,2-trifluoro-N-(2-(5-methoxy-1H-indol-3-yl)ethyl)-N-methylethan-1-amine (IV-40) was carried out according to FIG. 21. 5-methoxy-1H-indole (IV-40a) was reacted with oxalyl chloride (1.5 eq.) in diethyl ether at 0° C. for 30 min to form intermediate IV-40b in 84% yield. Reaction with 2,2,2-trifluoro-N-methylethan-1-amine (3 eq.) from 0° C. to room temperature over 90 min provided intermediate IV-40c in 86% yield. Reduction with LAH (6 eq.) in dioxane at 85-90° C. overnight provided crude product residue which was purified by flash column chromatography to provide the title compound in 69% yield; the structure was confirmed by ¹⁹F-NMR.

The HCl salt of IV-40 was prepared by dissolving IV-40 (free base) prepared above in methanol followed by treatment with HCl in diethyl ether.

### II. Testing

### Methods.

### Serotonin (5-HT) Receptor Pharmacodynamics

Binding affinity (Kᵢ) and functional potency and efficacy (EC₅₀ and EMAX) values of the compounds were measured. Receptor Affinity Assays: 5-HT₂ (A,B,C) receptor affinities were determined by radioligand competition binding as previously described (Canal, C. E., Cordova-Sintjago, T., Liu, Y., Kim, M. S., Morgan, D., and Booth, R. G., 2013, Molecular pharmacology and ligand docking studies reveal a single amino acid difference between mouse and human serotonin 5-HT_{2A} receptors that impacts behavioral translation of novel 4-phenyl-2-dimethylaminotetralin ligands, J Pharmacol Exp Ther 347, 705-716; Armstrong, J. L., Casey, A. B., Saraf, T. S., Mukherjee, M., Booth, R. G., and Canal, C. E., 2020, (S)-5-(2′-Fluorophenyl)-N,N-dimethyl-1,2,3,4-tetrahydronaphthalen-2-amine, a Serotonin Receptor Modulator, Possesses Anticonvulsant, Prosocial, and Anxiolytic-like Properties in an Fmr1 Knockout Mouse Model of Fragile X Syndrome and Autism Spectrum Disorder, ACS Pharmacol Transl Sci 3, 509-523).

### Radioligand Competition Binding

Radioligand competition binding was performed as previously described (Saraf, T. S., Felsing, D. E., Armstrong, J. L., Booth, R. G., and Canal, C. E., 2021, Evaluation of lorcaserin as an anticonvulsant in juvenile Fmr1 knockout mice, Epilepsy Res 175, 106677), with minor modifications, using human cloned 5-HT_{2A} receptors. Plasmids encoding human serotonin 5-HT_{2A} receptor cDNA were procured from the cDNA Resource Center. Human embryonic kidney cells (HEK293, ATCC CRL-1573) were grown in a cell incubator in 100 mm dishes with antibiotic-free Dulbecco's modified Eagle's medium containing 10% fetal bovine serum. Cells were transfected at ~85% confluency with 5-15 μg cDNA using TransIT-2020 reagent (Mirus Bio, Madison, Wisconsin). After approximately 48 hours, cell membrane was collected via centrifugation. For all experiments, serotonin (5-HT) hydrochloride was used as a positive control, and mianserin hydrochloride (10 μM) was used to define non-specific binding. Each cell membrane homogenate expressing 5-HT_{2A} receptors was incubated at room temperature in 96-well plates with [³H] Lysergic acid diethylamide ([³H]LSD), between 0.5 and 1 nM, in the absence or presence of test compounds in a buffer for 90 minutes. Following equilibration, each sample was filtered rapidly under vacuum through fiberglass filters presoaked in a buffer and washed by vacuum several times with an ice-cold buffer. Filters were soaked with scintillation fluid and counts per minute were detected using photodetectors (Microbeta Scintillation Counter). K_{d} for [³H]LSD was set to 0.78 nM and the IC₅₀ values were computed using nonlinear, least squares regression analyses and then converted to Kᵢ values using the Cheng-Prusoff equation (GraphPad Prism 9.0, San Diego, California). Data shown are the results from all experiments combined. As is known, G protein-coupled receptors, including the 5-HT_{2A} receptor, can exist in multiple conformations, and ligands, typically agonist ligands, have unique affinities for these conformations of the receptor (Kenakin, T. Theoretical Aspects of GPCR-Ligand Complex Pharmacology, Chem. Rev. 2017, 117, 1, 4-20). The competition binding data from some of the tested compounds fit best to a two-site fit Kᵢ model. However, for simplicity reasons, affinity of these compounds are reported herein as fitted to the single-site model. The data point for −10 samples is total specific binding (no compound present), and −4 was interpolated to complete the binding curves to 0 specific binding.

### Receptor Function Assays

5-HT₂ receptor-mediated Gq stimulation [phosphoinositide hydrolysis leading to the production of inositol phosphate 1 (IP1)]-canonical signaling pathways-were measured as previously described (Canal, C. E., Cordova-Sintjago, T., Liu, Y., Kim, M. S., Morgan, D., and Booth, R. G., 2013, Molecular pharmacology and ligand docking studies reveal a single amino acid difference between mouse and human serotonin 5-HT_{2A} receptors that impacts behavioral translation of novel 4-phenyl-2-dimethylaminotetralin ligands, J Pharmacol Exp Ther 347, 705-716; Canal, C. E., Morgan, D., Felsing, D., Kondabolu, K., Rowland, N. E., Robertson, K. L., Sakhuja, R., and Booth, R. G., 2014, A Novel Aminotetralin-Type Serotonin (5-HT)(2C) Receptor —Specific Agonist and 5-HT_{2A} Competitive Antagonist/5-HT_{2B} Inverse Agonist with Preclinical Efficacy for Psychoses, J Pharm Exp Ther 349, 533), for example, with a homogeneous time-resolved fluorescence (HTRF) capable microplate reader (e.g., Mithras LB 940, Berthold) using commercially-available kits employing Fluorescence Resonance Energy Transfer (FRET) technology (e.g., IP-One HTRF (Cisbio) kits). Briefly, CHO-K1 or HEK293 cells expressing a single serotonergic 5-HT₂ receptor subtype were incubated with test compounds and 5-HT or DOI (positive control) in stimulation buffer containing LiCl. After equilibration, the reaction was terminated with the donor and acceptor fluorescent conjugates in lysis buffer, and FRET was measured with a microplate reader (Berthold Mithras). Data were fit to non-linear curves to calculate potencies (e.g., EC₅₀) and efficacies (e.g., EMAX), relative to positive controls (e.g., serotonin).

### In Vitro Liver Metabolism (Rat Liver Microsomes, RLM)

Male Sprague-Dawley rat liver microsomes were purchased from XenoTech. The reaction mixture, minus NADPH, was prepared as described below. The test compound was added into the reaction mixture at a final concentration of 1 μM. The control compound, testosterone, was run simultaneously with the test compound in a separate reaction. The reaction mixture (without cofactor) was equilibrated in a shaking water bath at 37° C. for 5 minutes. The reaction was initiated by the addition of the cofactor, and the mixture was incubated in a shaking water bath at 37° C. Aliquots (150 μL) were withdrawn at 0, 5, 10, 20, 30, 60, and 120 minutes. Test compound and testosterone samples were immediately combined with 150 μL of ice-cold acetonitrile (ACN) containing 0.1% formic acid and internal standard to terminate the reaction. The samples were then mixed and centrifuged to precipitate proteins. All samples were assayed by LC-MS/MS using electrospray ionization. The peak area response ratio (PARR) of analyte to internal standard at each time point was compared to the PARR at time 0 to determine the percent remaining at each time point. Half-lives were calculated using GraphPad software, fitting to a single-phase exponential decay equation. The reaction composition is provided in Table 1.

TABLE 1

| | | |
|---|---|---|
| Liver Microsomes | 0.5 | mg/mL |
| NADPH (cofactor) | 1 | mM |
| Potassium Phosphate, pH 7.4 | 100 | mM |
| Magnesium Chloride | 5 | mM |
| Test compound | 1 | μM |

### Stability in Recombinant Human MAO-A

hrMAO-A and MAO control were purchased from XenoTech. The reaction mixture was prepared as described below. The test compound was added into the reaction mixture at a final concentration of 1 μM. The positive control, kynuramine (25 μM), was run simultaneously with the test compound in a separate reaction. The reaction mixture (without test compounds or kynuramine) was equilibrated in a shaking water bath at 37° C. for 5 minutes. The reaction was initiated by the addition of the test compound or kynuramine, and the mixture was incubated in a shaking water bath at 37° C. Aliquots (150 μL) of the test compound reaction mixture were withdrawn at 0, 5, 10, 20, 30,60, and 120 minutes. Aliquots (150 μL) of the positive control reaction mixture were withdrawn at 0 and 30 minutes. Test compound and kynuramine samples were immediately combined with 150 AL of ice-cold 100% acetonitrile containing 0.1% formic acid and internal standard (0.2 μM metoprolol) to terminate the reaction. The samples were then mixed and centrifuged to precipitate proteins. All samples were assayed by LC-MS/MS. The peak area response ratio (PARR) of analyte to internal standard at each time point was compared to the PARR at time 0 to determine the percent remaining at each time point. Half-lives were calculated using GraphPad software, fitting to a single-phase exponential decay equation. The reaction composition is provided in Table 2.

TABLE 2

| | | |
|---|---|---|
| hrMAO | 0.02 | mg/mL |
| Potassium Phosphate, pH 7.4 | 100 | mM |
| Magnesium Chloride | 5 | mM |
| Test compound | 1 | μM |

### Head-Twitch Response (HTR)

The HTR assay was performed in adult, male C57BL/6J mice, procured from the Jackson Laboratory (Bar Harbor, Maine), as previously described (Canal, C. E., and Morgan, D., 2012, Head-twitch response in rodents induced by the hallucinogen 2,5-dimethoxy-4-iodoamphetamine: a comprehensive history, a re-evaluation of mechanisms, and its utility as a model, Drug Test Anal 4, 556-576; Saraf, T. S., Felsing, D. E., Armstrong, J. L., Booth, R. G., and Canal, C. E., 2021, Evaluation of lorcaserin as an anticonvulsant in juvenile Fmr1 knockout mice, Epilepsy Res 175, 106677). Mice were housed in standard laboratory cages with ad libitum access to food and water, and were acclimated to the vivarium for at least one week prior to testing in a procedure room. On the day of testing, mice were acclimated to the procedure room in their home cages for ≥60 min before administration of test compounds. Test articles were administered at 10 mg/kg by oral gavage and mice were placed immediately thereafter in a clear, polycarbonate box (46×20×20 cm), and HTRs were counted for 30 consecutive minutes, using hand-held tally counters, by two trained observers who were blind to treatment.

### Pharmacokinetic (PK) Studies in Mice

PK profiles were established in adult, male C57BL/6J (Jackson Laboratories). Mice were housed in standard laboratory cages with ad libitum access to food and water, and were acclimated to the vivarium for at least one week prior to testing in a procedure room. In a typical experiment, mice were administered 10 mg/kg of test compound by oral gavage and plasma and brain samples were collected at 10, 30, and 120 min with n-3 for each time point. Quantification of the parent molecule was done using LC-MS/MS method (LLOQ, 0.9 nM).

### Results.

A summary of testing results for Examples 1-13, 15-16, and 18-20 are presented in Tables 3-4.

TABLE 3

| | | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|---|
| Compound | | I-3 | II-2 | II-3 | II-6 | II-10 | II-12 | II-15 | II-19 | II-20 |
| RLM, t_{1/2} (min) | | — | — | — | — | Not | — | — | 3.95 | 7.63 |
| | | | | | | detected | | | | |
| MAO-A (min) | | — | — | — | — | — | — | — | — | >120 |
| 5-HT_{2A} − Kᵢ (nM) | | — | — | — | 12 (LSD)^{a} | ND^{b} | — | >3000 | ND^{b} | ND^{b} |
| 5-HT_{2A} efficacy | 500 nM | −34.2 ± | 1.4 ± | −29.7 ± | −7.7 ± 11.6 | — | 19.9 ± | −24.11 ± | — | — |
| relative to | | 28.5 | 10.5 | 12.7 | | | 14.3 | 10.4 | | |
| maximal 5-HT | 50 μM | 43.9 ± | 91.8 ± | 57.9 ± | −3.1 ± 9.6 | — | 30.3 ± | −36.9 ± | — | — |
| response | | 11.9 | 18.1 | 30.1 | | | 16.1 | 8.7 | | |
| 5-HT_{2A} | EC₅₀ (nM) | — | 596 | — | — | — | — | — | — | — |
| | Eₘₐₓ (%) | — | 79 | — | — | — | — | — | — | — |

| HTR (per min) | — | — | — | <1 | | — | — | — | — | — |
|---|---|---|---|---|---|---|---|---|---|---|
| PK data | — | — | — | Plasma | Brain | — | — | — | — | — |
| 10 min (μM) | | | | 0.82-7.76 | 1.55-12.6 | | | | | |
| 30 min (μM) | | | | 0.39-3.14 | 0.72-4.71 | | | | | |
| 120 min (μM) | | | | 0.007-0.44 | 0.021-1.13 | | | | | |

^{a}one-site fit Kᵢ model
^{b}Incomplete competition at high concentrations; Kᵢ not determined (ND)

TABLE 4

| | | Ex. 10 | Ex. 11 | Ex. 12 | Ex. 13 | Ex. 15 | Ex. 16 | Ex. 18 | Ex. 19 | Ex. 20 |
|---|---|---|---|---|---|---|---|---|---|---|
| Compound | | II-21 | III-2 | III-3 | III-6 | III-12 | III-13 | IV-3 | IV-21 | IV-40 |
| RLM, t_{1/2} (min) | | — | — | — | — | — | 4.5 | 13.1 | — | 4.55 |
| 5-HT_{2A} − Kᵢ (nM) | | — | — | — | 12 (LSD)^{a} | — | — | — | — | — |
| 5-HT_{2A} efficacy | 500 nM | 9.2 ± | 58.3 ± | 29.2 ± | 53.5 ± | 17.8 ± | — | — | 29.5 ± | — |
| relative to | | 16 | 12.8 | 6.5 | 24.2 | 32 | | | 15.6 | |
| maximal 5-HT | 50 μM | −25.4 ± | 63.7 ± | 54.8 ± | 60.2 ± | 58.8 ± | — | — | 79.9 ± | — |
| response | | 10.8 | 7.7 | 7.6 | 8.4 | 8.2 | | | 11.7 | |
| 5-HT_{2A} | EC₅₀ (nM) | — | 383 | — | — | — | — | — | >3000 | — |
| | Eₘₐₓ (%) | — | 100^{b} | — | — | — | — | — | 100^{b} | — |
| 5-HT_{2B} | EC₅₀ (nM) | — | 96 | — | — | — | — | — | 394 | — |
| | Eₘₐₓ (%) | — | 65 | — | — | — | — | — | 62 | |

| HTR (per min) | — | — | — | <1 | | — | — | — | — | — |
|---|---|---|---|---|---|---|---|---|---|---|
| PK data | — | — | — | Plasma | Brain | — | — | — | — | — |
| 10 min (μM) | | | | 0.17-0.62 | 0.2-1.3 | | | | | |
| 30 min (μM) | | | | 0.072-0.18 | 0.12-0.42 | | | | | |
| 120 min (μM) | | | | 0.002-0.016 | 0.003-0.02 | | | | | |

^{a}one-site fit Kᵢ model
^{b}Eₘₐₓ was constrained to 100% for curve fitting

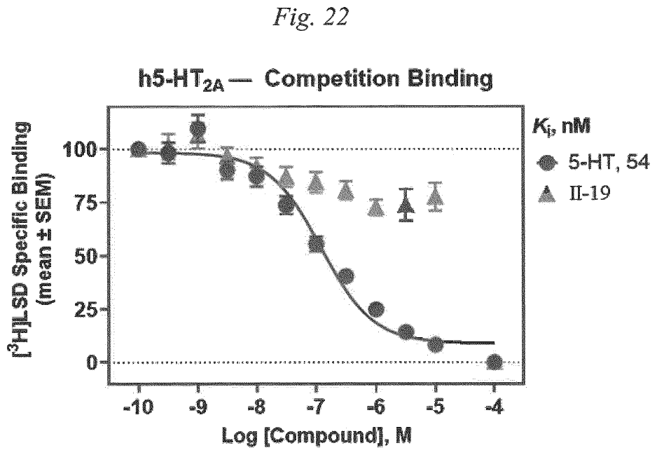

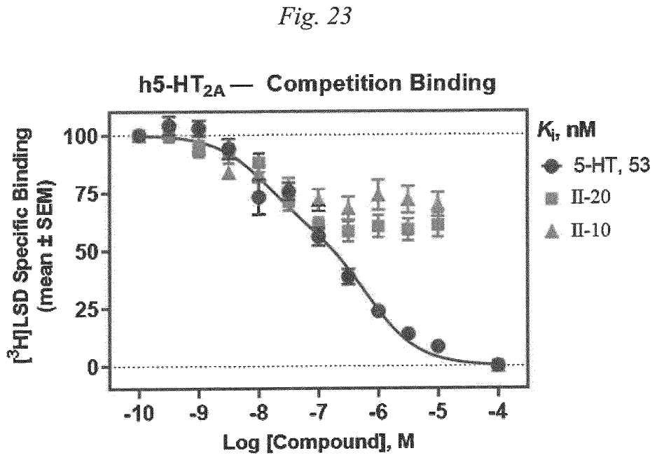

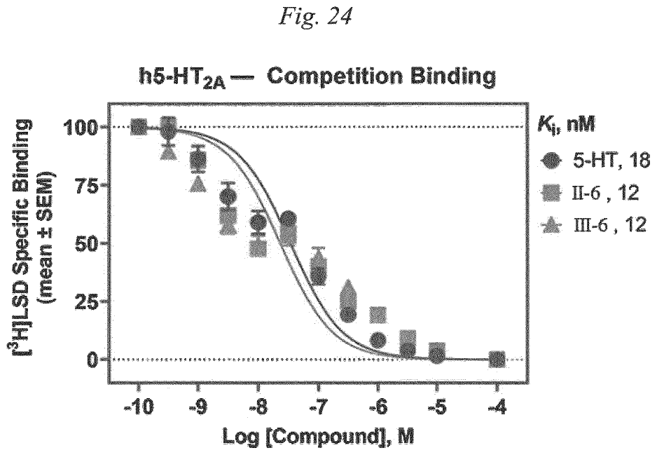

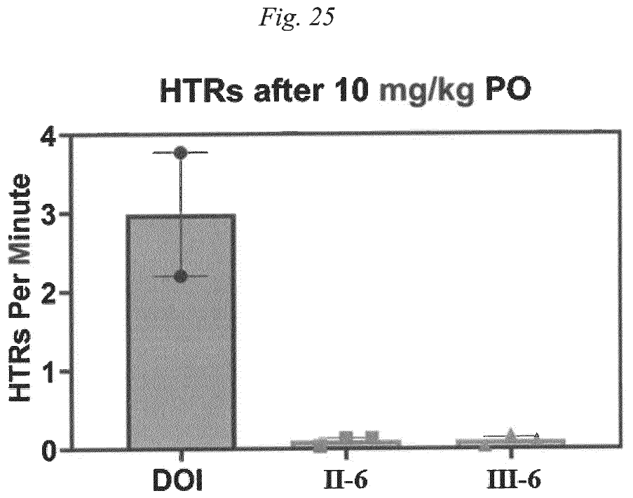

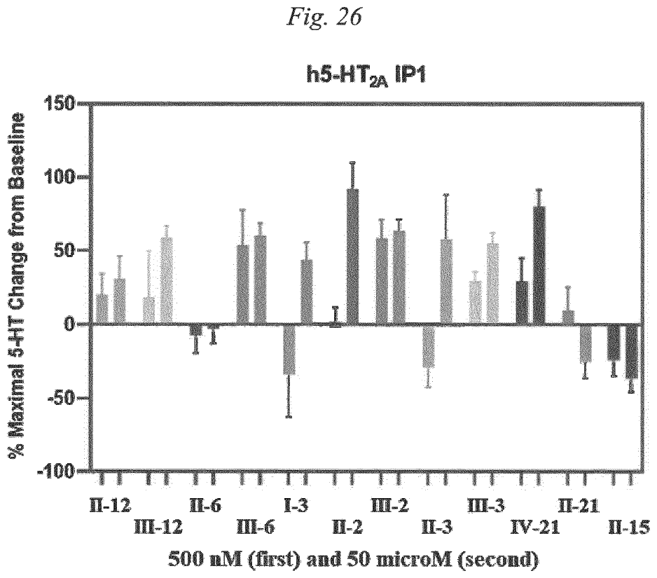

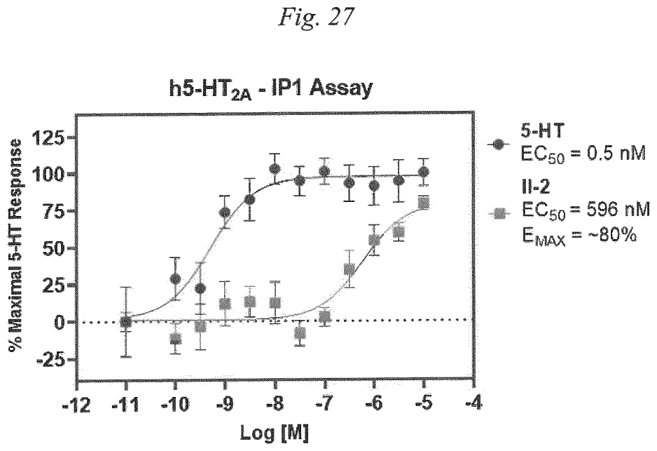

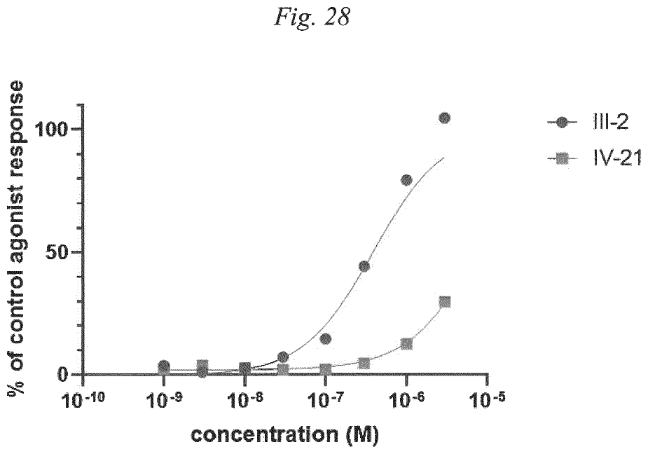

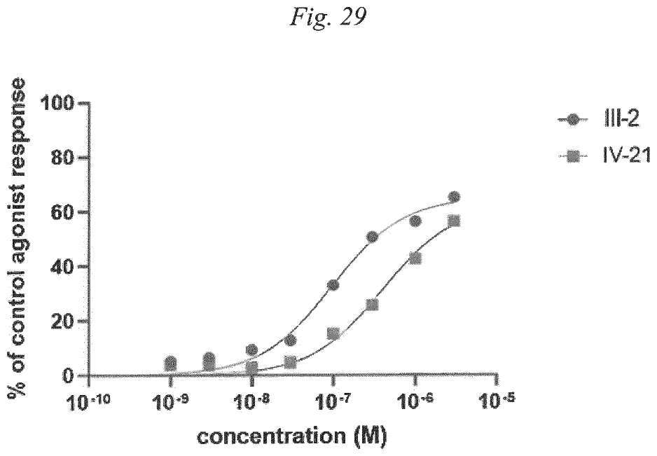

The results from the human serotonin 5-HT_{2A} receptor radioligand ([³H]LSD) competition binding assay (FIGS. 22-24), head-twitch response (FIG. 25), 5-HT_{2A} receptor function assay (FIGS. 26-28), and 5-HT_{2B} receptor function assay (FIG. 29) are also represented graphically.

The in vitro 5-HT_{2A} receptor binding and function and in vivo 5-HT_{2A}-dependent HTR pharmacology results provided unexpected discoveries. Notably, the compounds II-10, II-19, and II-20 showed only partial competition for [³H]LSD-labeled 5-HT_{2A} receptors, up to concentrations of 10 μM in radioligand competition binding assays. These data suggest that these compounds bind unique conformations or populations of 5-HT_{2A} receptors relative to LSD or bind 5-HT_{2A} receptors at different sites, e.g., bind to allosteric sites on 5-HT_{2A} receptors (See Kenakin, Terry. A pharmacology primer: techniques for more effective and strategic drug discovery. Academic Press, 2018). In support of these conclusions, the structurally-related compounds, II-6 and III-6 showed complete competition for [³H]LSD-labeled 5-HT_{2A} receptors with high affinities; however, the competition binding curves exhibited multiple slopes, suggesting the compounds bound different 5-HT_{2A} receptor conformations with different affinities.

The results from tests of 5-HT_{2A} functional activities of this series of compounds were also unexpected in the context of the unusual results observed from competition binding assays. For example, III-12, III-6, II-2, II-3, III-2, III-3, and IV-21 showed, on average, greater than 50% agonist efficacy at 5-HT_{2A} receptor at 50 μM concentrations. Complete dose-response 5-HT_{2A} and 5HT_{2B} functional assays (measuring IP1 formation) were conducted for III-2 and IV-21 by a second, independent laboratory. III-2 was a low potency, full agonist in the 5-HT_{2A} functional assay; calculated EC₅₀ was 383 nM. These results are consistent with agonist effects observed in the independent screening test. III-2 was also a partial agonist at 5HT_{2B} receptors, with an EC₅₀ of 96 nM. IV-21 was a low potency, EC₅₀>3 μM, agonist at 5-HT_{2A}, though, the dose-response curve had not plateaued at the highest concentration tested, 3 μM. These results are consistent with agonist effects observed in the independent screening test. IV-21 was also a partial agonist at 5HT_{2B} receptors, with an EC₅₀ of 394 nM.

II-15 showed inverse agonist efficacy at 5-HT_{2A} receptors at both 500 nM and 50 μM concentrations. However, II-15 showed very low (>3 μM) 5-HT_{2A} affinity in a competition binding assay with [125I]DOI, another 5-HT_{2A} agonist radioligand; these data suggest II-15, like other compounds in this series, has unusual binding to 5-HT_{2A} receptors labeled with classic psychedelic 5-HT_{2A} agonists. The inverse agonist activity of II-15 may translate to antipsychotic-like activity, as evidenced by the FDA-approved medicine Nuplazid® (pimavanserin). In conclusion, these compounds have measurable activity at 5-HT_{2A} receptors.

In the in vivo 5-HT_{2A} receptor-dependent HTR assay in mice, II-6 and III-6 did not elicit a HTR after mg/kg PO administration. This is despite III-6 exhibiting agonist activity at 5-HT_{2A} receptors in vitro. Experimenters, however, noted qualitative behavioral changes elicited by the compounds, including reduced locomotion and “hiccup” responses, suggesting the compounds were orally active. Pharmacokinetic tests of plasma and brain concentrations after 10 mg/kg PO administration of these compounds confirmed that they were brain-penetrant. When 5-HT_{2A} agonists do not elicit the HTR, it is often due to activity of the compounds at additional targets that suppress the HTR (see Canal, C. E., and Morgan, D., 2012, Head-twitch response in rodents induced by the hallucinogen 2,5-dimethoxy-4-iodoamphetamine: a comprehensive history, a re-evaluation of mechanisms, and its utility as a model, Drug Test Anal 4, 556-576). Indeed, the serotonergic psychedelic N,N-diethyltryptamine (DET) is active orally in humans (Shulgin, Alexander Theodore, and Ann Shulgin. TIHKAL: the continuation. Vol. 546. Berkeley: Transform press, 1997; also see Erowid. (2002 March 1). DET Erowid.org. Retrieved Jul. 21, 2022), but was inactive at eliciting the HTR after 10 mg/kg PO administration. Similar to II-6 and III-6, experimenters noted qualitative behavioral changes after mice were administered DET PO. These included accelerated breathing, hyperlocomotion, hiccup responses, and chewing movements of mouth.

In summary, the tested compounds show unique activities at 5-HT_{2A} receptors, in vitro and in vivo. For example, III-6 shows potent affinity at 5-HT_{2A} receptors, where it behaved as an agonist. However, III-6 did not elicit the 5-HT_{2A}-dependent HTR after PO administration at 10 mg/kg, despite being detected in the brain at concentrations that would be expected to occupy 5-HT_{2A} receptors to elicit the HTR. Qualitative observations noted that III-6 elicited “hiccup” like responses and caused hypolocomotion after 10 mg/kg PO administration. Thus, this compound might be a non-psychedelic 5-HT_{2A} agonist.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Accordingly, the preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. A compound of Formula (I), or a pharmaceutically acceptable salt,
stereoisomer, tautomer, solvate, polymorph, or prodrug thereof, 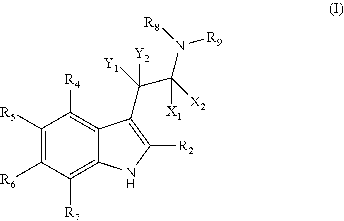
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is;
R₄ is selected from the group consisting of hydrogen, deuterium, hydroxyl, unsubstituted or substituted alkoxy, and —OPO₃H₂;
R₈ is selected from the group consisting of hydrogen, unsubstituted alkoxy, alkoxy substituted with one or more deuterium, unsubstituted alkylthio, alkylthio substituted with one or more deuterium, —OR_{f}, and —SR_{f};
R₆ and R₇ are independently selected from the group consisting of hydrogen, deuterium, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₆ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f};
or alternatively R₅ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine; and
with the provisos that (i) R₉ is not —CH₂CF₃ when X₁, X₂, Y₁, Y₂, R₂, R₄, R₅, R₆, and Ry are hydrogen and R₈ is hydrogen or methyl, and (ii) R₈ and R₇ are not joined with the nitrogen atom attached thereto to form a 4,4-difluoropiperidinyl group when X₁, X₂, Y₁, Y₂, R₂, R₄, R₅, R₆, and
R₇ are hydrogen.

2-21. (canceled)

22. The compound of claim 1, having a structure of Formula (II), or a pharmaceutically acceptable salt, stereoisomer, tautomer, solvate, polymorph, or prodrug thereof, 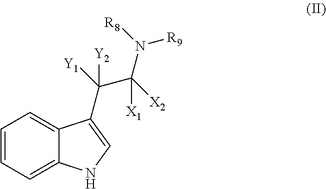
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f};
or alternatively R₅ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₈ and R₉ comprises the fluoroalkyl group, R_{f}, or R₅ and R₉ together with the nitrogen atom attached thereto are joined to form the heterocycloalkyl substituted with at least one fluorine; and
with the provisos that (i) R₉ is not —CH₂CF₃ when X₁, X₂, Y₁, and Y₂ are hydrogen and R₈ is hydrogen or methyl and (ii) R₈ and R₇ are not joined with the nitrogen atom attached thereto to form a 4,4-difluoropiperidinyl group when X₁, X₂, Y₁, and Y₂ are hydrogen.

23. The compound of claim 22, wherein X₁, X₂, Y₁, and Y₂ are hydrogen.

24. The compound of claim 22, wherein R₈ is hydrogen or an unsubstituted C₁-C₆ alkyl.

25. The compound of claim 22, wherein R₈ is R_{f}.

26. The compound of claim 25, wherein H^{x} is hydrogen and n is 2.

27. The compound of claim 22, wherein R₉ is R_{f}.

28. The compound of claim 27, wherein H^{x} is hydrogen and n is 2.

29-30. (canceled)

31. The compound of claim 22, which is selected from the group consisting of 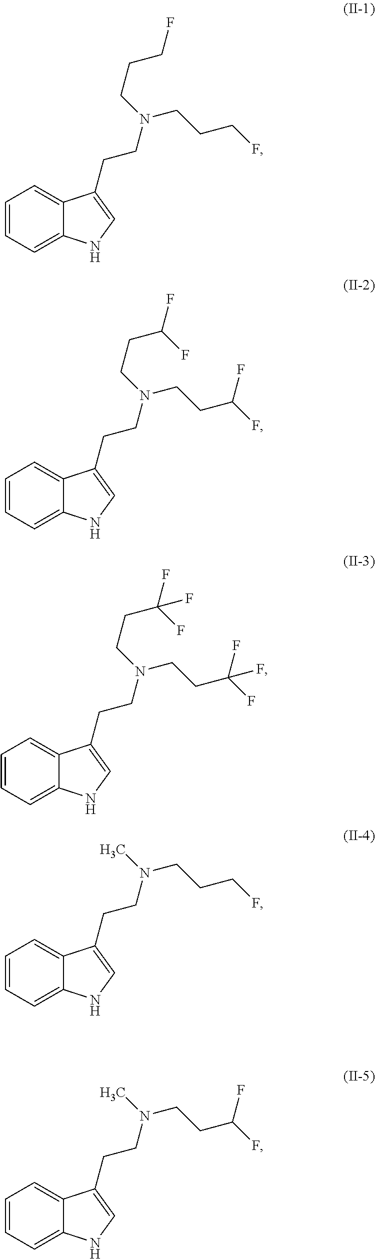 [SMILES: FCCCN(CCCF)CCc1c[nH]c2ccccc12 ; FC(F)CCN(CCc1c[nH]c2ccccc12)CCC(F)F ; FC(F)(F)CCN(CCc1c[nH]c2ccccc12)CCC(F)(F)F ; CN(CCCF)CCc1c[nH]c2ccccc12 ; CN(CCc1c[nH]c2ccccc12)CCC(F)F] 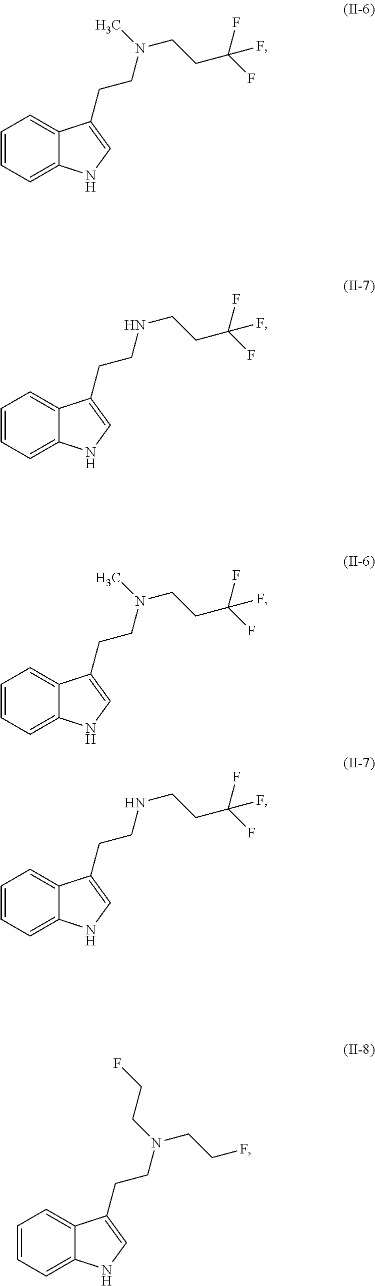 [SMILES: CN(CCc1c[nH]c2ccccc12)CCC(F)(F)F ; FC(F)(F)CCNCCc1c[nH]c2ccccc12 ; CN(CCc1c[nH]c2ccccc12)CCC(F)(F)F ; FC(F)(F)CCNCCc1c[nH]c2ccccc12 ; FCCN(CCF)CCc1c[nH]c2ccccc12] 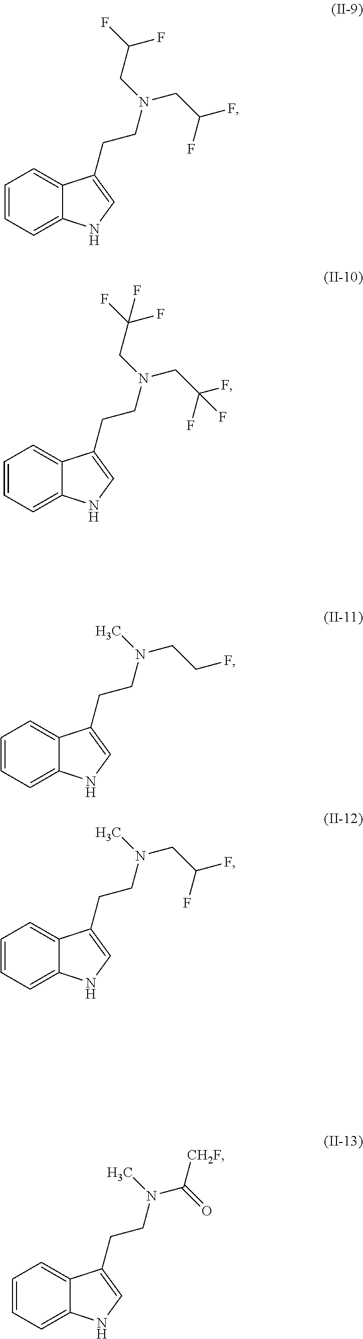 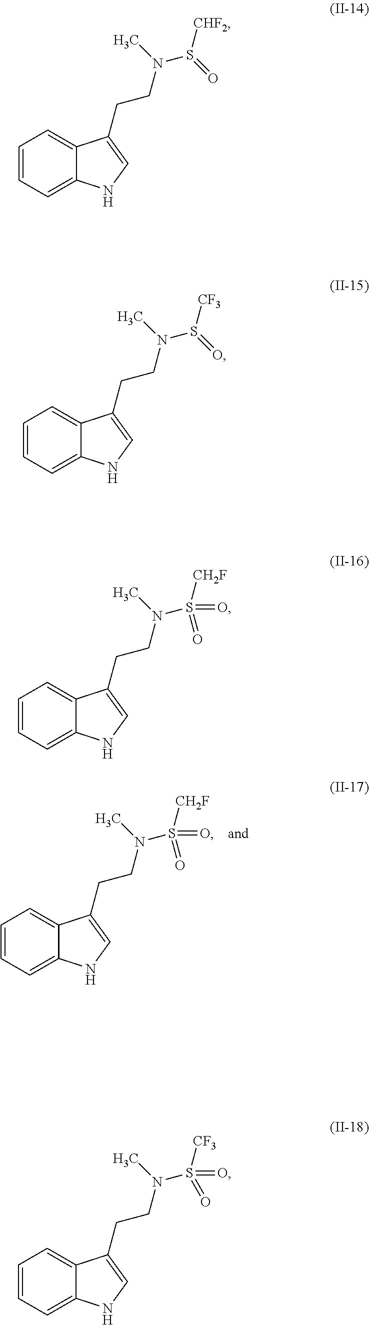
or a pharmaceutically acceptable salt, stereoisomer, tautomer, solvate, polymorph, or prodrug thereof.

32. The compound of claim 1, having a structure of Formula (III), or a pharmaceutically acceptable salt, stereoisomer, tautomer, solvate, polymorph, or prodrug thereof, 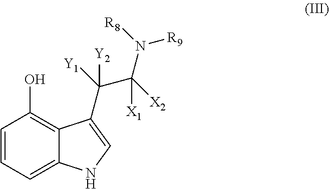
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
Rₐ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f},
or alternatively R₅ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH 2)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₈ and R₉ comprises the fluoroalkyl group, R_{f}, or R₈ and R₉ together with the nitrogen atom attached thereto are joined to form the heterocycloalkyl substituted with at least one fluorine.

33. The compound of claim 32, wherein X₁, X₂, Y₁, and Y₂ are hydrogen.

34. The compound of claim 32, wherein R₈ is hydrogen or an unsubstituted C₁-C₆ alkyl.

35. The compound of claim 32, wherein R₈ is R_{f}.

36. The compound of claim 35, wherein H^{x} is hydrogen and n is 2.

37. The compound of claim 32, wherein R₉ is R_{f}.

38. The compound of claim 37, wherein H^{x} is hydrogen and n is 2.

39-40. (canceled)

41. The compound of claim 32, which is selected from the group consisting of 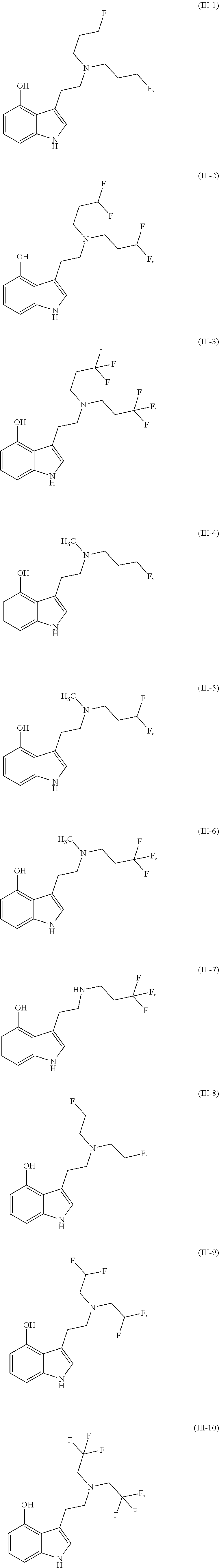 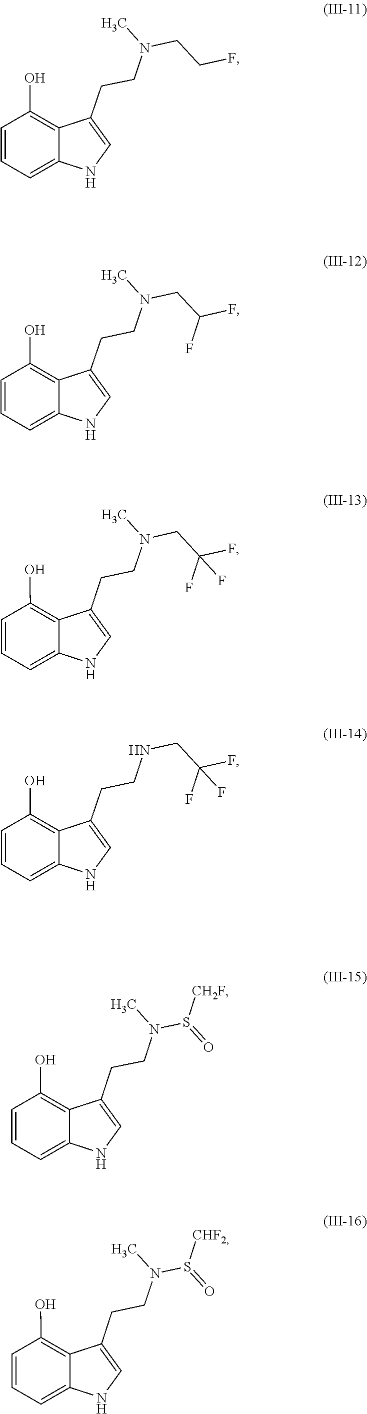 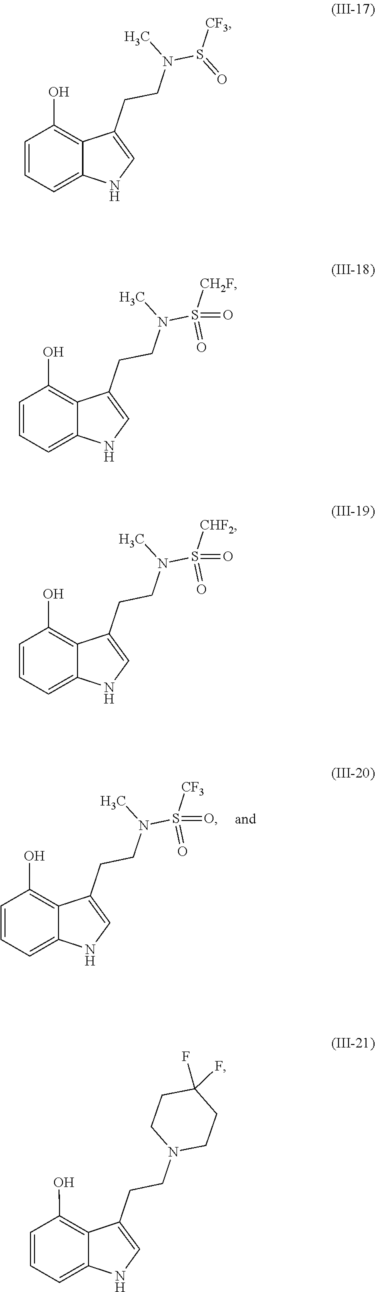
or a pharmaceutically acceptable salt, stereoisomer, tautomer, solvate, polymorph, or prodrug thereof.

42. The compound of claim 1, having a structure of Formula (IV), or a pharmaceutically acceptable salt, stereoisomer, tautomer, solvate, polymorph, or prodrug thereof, 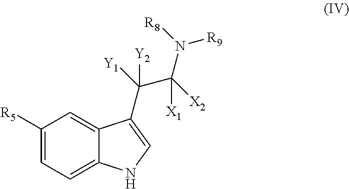
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of unsubstituted alkoxy, alkoxy substituted with one or more deuterium, and —OR_{f};
Rₐ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f};
or alternatively R₈ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂) CHF₂, and —(CH^{x}₂)ₙCF₃, wherein n is 0 to 3, and each H^{x} is independently hydrogen or deuterium;
wherein at least one of R₅, R₈, and Ry comprises the fluoroalkyl group, R_{f}, and/or R₈ and R₉ together with the nitrogen atom attached thereto are joined to form the heterocycloalkyl substituted with at least one fluorine.

43-55. (canceled)

56. The compound of claim 1, having a structure of Formula (V), or a pharmaceutically acceptable salt, stereoisomer, tautomer, solvate, polymorph, or prodrug thereof, 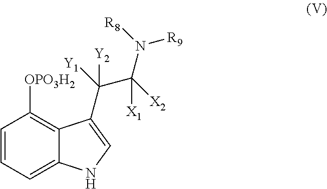
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₈ is selected from the group consisting of hydrogen, unsubstituted alkyl, an alkyl substituted with one or more deuterium, and R_{f};
R₉ is selected from the group consisting of unsubstituted alkyl, an alkyl substituted with one or more deuterium, R_{f}, —S(O) R_{f}, and —S(O)₂R_{f},
or alternatively R₅ and R₉ together with the nitrogen atom attached thereto are optionally joined to form a heterocycloalkyl substituted with at least one fluorine; and
R_{f} is a fluoroalkyl group, with each R_{f} being independently selected from the group consisting of —(CH^{x}₂)ₙCH₂F, —(CH^{x}₂)ₙCHF₂, and —(CH^{x}₂)ₙCF₃, wherein n is 0 to 3, and each H^{x} is independently hydrogen or deuterium;
wherein at least one of R₈ and R₉ comprises the fluoroalkyl group, R_{f}, or R₈ and R₉ together with the nitrogen atom attached thereto are joined to form the heterocycloalkyl substituted with at least one fluorine.

57-65. (canceled)

66. A pharmaceutical composition comprising:
the compound of claim 1, or a pharmaceutically acceptable salt, stereoisomer, tautomer, solvate, polymorph, or prodrug thereof; and
a pharmaceutically acceptable vehicle.

67-68. (canceled)

69. A method of treating a subject with a disease or disorder associated with a serotonin 5-HT₂ receptor, comprising:
administering to the subject a therapeutically effective amount of a compound of claim 1, or a pharmaceutically acceptable salt, stereoisomer, tautomer, solvate, polymorph, or prodrug thereof.

70. The method of claim 69, wherein the disease or disorder is a neuropsychiatric disease or disorder or an inflammatory disease or disorder.

71. The method of claim 69, wherein the disease or disorder is a central nervous system (CNS) disorder.

72. The method of claim 71, wherein the central nervous system (CNS) disorder is at least one selected from the group consisting of major depressive disorder (MDD), treatment-resistant depression (TRD), post-traumatic stress disorder (PTSD), bipolar and related disorders, obsessive-compulsive disorder (OCD), generalized anxiety disorder (GAD), social anxiety disorder, a substance use disorder, an eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, a paraphilic disorder, sexual dysfunction, peripheral neuropathy, and obesity.

73-90. (canceled)
